(19) United States
(12) Patent Application Publication
(10) Pub. No.: US 2020/0224236 A1
(43) Pub. Date: Jul. 16, 2020

(54) Microbial Production of Protein and PHB by Alcohol Utilizing Bacteria

(71) Applicant: KnipBio, Inc., Lowell, MA (US)

(72) Inventors: Lawrence F. Feinberg, Lowell, MA (US); Daniel R. Smith, Lowell, MA (US); Martha A. Sholl, Lowell, MA (US); Sophana Sopha, Lowell, MA (US); Bonnie D. McAvoy, Lowell, MA (US); Catherine J. Pujol-Baxley, Lowell, MA (US)

(21) Appl. No.: 16/467,471

(22) PCT Filed: Dec. 2, 2017
(86) PCT No.: PCT/US2017/064375

§ 371 Date: Jun. 6, 2019

Related U.S. Application Data
(60) Provisional application No. 62/432,185, filed on Dec. 9, 2016.

(51) Int. Cl. C12P 21/00; C12N 1/20; C12P 7/62; A23K 10/18; A23K 10/12; A23L 33/135

(52) U.S. Cl. CPC C12P 21/00; C12N 1/20; C12P 7/625; A23V 2002/00; A23K 10/12; A23L 33/135; A23K 10/18

(57) ABSTRACT

Microorganisms and methods are provided for producing biomass that includes PHB and protein in weight ratios and polymer lengths that are beneficial in feed and nutritional supplement compositions. The compositions also may be used for improvement in feed compositions that improve survivability of livestock and aquaculture species.

### CROSS-REFERENCE TO RELATED APPLICATIONS

This application claims the benefit of U.S. Provisional Application No. 62/432,185, filed Dec. 9, 2016, which is incorporated herein by reference in its entirety.

### FIELD OF THE INVENTION

The invention relates to microorganisms and methods for producing biomass with a high ratio of protein to polyhydroxyalkanoate, and use of such biomass in feed and nutritional supplement compositions.

### BACKGROUND

Approaches to address disease to improve yields on farms is a timeless goal. Antibiotics have been a potent weapon on this front but given the general over-use that has led to further complications and the particular challenges for aqueous environments in aquaculture, alternatives to disease mitigation should be actively sought (Defoirdt, et al., (2011) Curr Opin Microbiol 14:251-258; Burridge, et al. (2010) Aquaculture 306:7-23). Organic acids have been described as capable of exhibiting bacteriostatic and bactericidal properties towards pathogenic bacteria (Ricke (2003) Poult Sci 632-639; Vazquez, et al (2005) Aquaculture 245:149-161; Wang, et al. (2008) Aquaculture 281:1-4; Ng, et al. (2015) Aquaculture 449:69-77; Romano, et al. (2015) Aquaculture 435:228-236). Their mode of action results in exhausting the cell metabolism therefore reducing cell growth and even leading to cell death (Hismiogullari, et al. (2008) J Anim Vet Adv 7:681-684).

Several types of short-chain fatty acids (SCFA) dosed at ˜2 g/L were shown to double the survival of the brine shrimp Artemia franciscana test specimens (Defoirdt, et al. (2006) Aquaculture 261:804-808). However, the use of SCFA may not be as suitable for aquaculture since these compounds are highly soluble in water. An alternative was found in the form of the bacterial storage polymer poly-1-hydroxybutyrate (PHB) (Defoirdt, et al. (2009) Biotechnol Adv 27:680-685). This compound serves as an intracellular energy and carbon reserve for bacteria (Tokiwa, et al. (2004) Biotechnol Lett 26:1181-1189), as well as a protectant against oxidative stress (Koskimiki, et al. (2016) Nat Chem Biol 12:332-338). It is insoluble in water and has been shown to be biologically degradable into β-hydroxybutyric acid (Bonartsev, et al. (2007) Commun Curr Res Educ Top Trends Appl Microbiol 295-307). The latter can exhibit growth inhibition towards certain pathogens such as Vibrio sp. (Seghal, et al. (2016) Npj Biofilm Microbiomes 2:16002) or Edwardsiella ictaluri (Situmorang, et al. (2016) Vet Microbiol 182: 44-49), protect A. franscicana like other SCFA do (Defoirdt, et al. (2007) Trends Biotechnol 25:472-497) and is a potential immunostimulant against Bacillus in tilapia (Suguna, et al. (2014) Fish and Shellfish Immunnol 36:90-97). As such, if PHB is supplemented through the feed and subsequently degraded in the gastrointestinal tract of aquaculture organisms, the locally released PHB oligomers may induce their beneficial effects. In several experiments with A. fransiscana, this approach increased the survival up to 73% upon infection with the pathogen Vibrio campbellii (Halet, et al. (2007) FEMS Microbiol Ecol 60:363-369); Defoirdt, et al. (2007) Environ Microbiol 9:445-452).

Literature supports several examples of PHB exhibiting positive influence in several aquatic animal species (Suguna, et al. (2014) Fish & Shellfish Immunol 36:90-97; Najdegerami, et al. (2015) Aquac Nutr doi: 10.1111/anu.12386). Najdegerami, et al. tested juvenile European sea bass at several doses of PHB inclusion rates, and the effects on the gut bacterial community composition were observed. The diets supplemented with 2% and 5% purified PHB (w/w) induced a gain of the initial fish weight with a factor 2.4 and 2.7, respectively, relative to a factor 2.2 in the normal feed treatment (De Schryver, et al. (2010) Appl Microbiol Biotechnol 86:1535-1541). Simultaneously, these treatments showed the highest bacterial range weighted richness in the fish intestine. Based on molecular analysis, higher dietary PHB levels induced larger changes in the bacterial community composition and it was interpreted that PHB can have a beneficial effect on fish growth performance and that the intestinal bacterial community structure may be closely related to this phenomenon.

PHB was provided to Siberian sturgeon fingerlings at concentrations of 2% and 5%, and the gastrointestinal tract microbial community was tracked. Diets containing PHB were observed to lead to greater species richness with the maximum found at 2% purified PHB. Siberian sturgeon fed PHB containing diets in general had poorer feed conversion ratios, seemingly significantly improved rates of survival and enhanced growth when fed 2%-containing PHB. (Najdegerami, et al. (2012) FEMS Microbiol Ecol 79:25-33)

A similar phenomenon was observed in penaeid shrimp. (Laranja, et al. (2014) Vet Microbiol 173:310-317) PHB accumulating mixed bacterial culture (mBC; 48.5% PHB on cell dry weight) and two PHB accumulating bacterial isolates, Bacillus sp. JL47 (54.7% PHB on cell dry weight) and Bacillus sp. JL1 (45.5% PHB on cell dry weight), were obtained from a Philippine shrimp culture pond and investigated for their capacity to improve growth, survival and robustness of Penaeus monodon post-larvae (PL). Shrimp PL and shrimp PL30 were provided PHB containing bacterial cultures in the feed for 30 days, followed by a challenge with pathogenic Vibrio campbellii. Prior to the pathogenic challenge, growth and survival were higher for shrimp receiving the PHB accumulating bacteria as compared to shrimp receiving diets without bacterial additions. After exposure to the pathogenic challenge, the shrimp fed PHB accumulating bacteria showed a higher survival as compared to non-treated shrimp, suggesting an increase in robustness for the shrimp. Similar effects were observed when shrimp PL30 were provided with the PHB accumulating bacterial cultures during a challenge with pathogenic V. campbellii through the water. The authors tested exposure to lethal ammonia stress but observed no significant difference between PHB accumulating bacteria-fed shrimp and non-PHB treated shrimp.

Methylobacterium extorquens is a naturally occurring bacterium found in nature as a leaf symbiont. In addition to several interesting growth features of this microbe, M. extorquens produces PHB as an energy storage molecule and/or as a physiological response to stress (Valentin & Steinbuchel (1993) Appl Microbiol Biotechnol 39:309-317). Historically, much effort has been invested in producing maximum levels of PHB as a precursor for biodegradable plastics (Bourque, et al. (1995) Appl Microbiol Biotechnol 44:367-376). As a cell's carbon budget is always constrained, production of PHB can cause carbon to flux away from products of interest including carotenoids, amino acids or general protein content. There is a need for a cell with lower PHB production, and ideally, higher content of protein and other organic compounds of interest. The ability to manipulate the ratio of protein:PHB in order to hit an optimum between protein content (e.g., >65%) and prebiotic effect would be highly desirable.

By decreasing unwanted carbon utilization, one can potentially increase growth rate, decrease carbon usage towards unwanted by-products, and increase carbon availability for production of desired products. Additionally, by decreasing or removing carbon products that accumulate to more than 1% of the total biomass, such as PHB or an exopolysaccharide (Kim, et al. (2003) World J Microbiol Biotechnol 19:357-361), one may effectively increase the content of protein or lipid that is useful in certain formulations of single cell protein sources for human or animal food.

Cells can have several forms of PHA/PHB, including storage PHB, medium chain PHB, and protein conjugated PHB (Reussch (2014) Int J Mol Sci 14:10727-48). Respectively, these forms range in size from 10,000-10,000,000, 100-200, to 10-20 residues per polymer chain. Storage PHB is found within protein bound granules in the cytoplasm of many bacteria. These proteins include the phasin coat proteins, PHB polymerases, PHB depolymerases, regulatory proteins, and granule organizing proteins such as PhaM (Jendrossek, et al. (2014) Enviro Microbiol 16:2357-73).

PHB polymers longer than 6-12 residues are insoluble in water (Reussch (2014) Int J Mol Sci 14:10727-48; Focarete, et al. (1999) Macromolecules 32:4184-4818) and thus are useful for aquaculture feed over soluble organic acids such as butyrate. The ability to control not only the amount, but also the average length of PHB polymer, is of importance to maximize the amount of organic acid available to the organism. Shorter water insoluble polymers of PHA/PHB should be more fully cleaved in the gut by chemical or enzymatic digestion into more readily available and active organic acid compounds (Silva, et al. (2016) J World Aquaculture Soc 47:508-18; Hoseinifar, et al. (2017) Aquaculture Res 48:1380-91).

The amount and average length of storage PHB is affected by environmental conditions, carbon and nitrogen sources, total carbon flux, and the relative activities of PHB polymerases and depolymerases (Anderson, et al. (1990) Microbiol Rev 54:450-472). Additionally, the overexpression of native depolymerases or expression of heterologous PHB digesting enzymes obtained from organisms that naturally degrade PHB (Sugiyama, et al. (2004) Cur Microbiol 48:424-7; Hadrick, et al. (2001) J Biol Chem 276:36215-24) should reduce the amount and average polymer length of PHB resulting in a superior feed with more bioavailable SCFA.

### BRIEF SUMMARY OF THE INVENTION

Microorganisms and methods are provided herein for production of polyhydroxyalkanoate (PHA) (e.g., poly-β-hydroxybutyrate (PHB)) and protein, feed and nutritional supplement compositions, and improvement of survivability of animals by consumption of the feed compositions.

In one aspect, non-naturally occurring microorganisms are provided. In some embodiments, the non-naturally microorganisms produce about 1% to about 99.9% less of a PHA product by weight and about 1% to about 250% more protein by weight than the parent microorganism from which the non-naturally occurring microorganism is derived. In some embodiments the non-naturally occurring microorganism may produce PHA and protein in a weight ratio of about 1:1000 to about 3:1, or about 1:1000 to about 1:6. In some embodiments, the PHA product produced by the non-naturally occurring microorganism is PHB.

In some embodiments, the non-naturally occurring microorganism is of the genus Methylomonas, Methylobacter, Methylococcus, Methylosinus, Methylocyctis, Methylomicrobium, Methanomonas, Methylophilus, Methylobacillus, Methylobacterium, Hyphomicrobium, Xanthobacter, Bacillus, Paracoccus, Nocardia, Arthrobacter, Rhodopseudomonas, Pseudomonas, Candida, Hansenula, Pichia, Torulopsis, Vibrio, Escherichia, Alcaligenes, Ralstonia, Rhodobacter, Saccharomyces, Cupriavidus, Sinorhizobium, Mucor, Bradyrhizobium, Yarrowia, Azotobacter, Synechocystis, Rhodotorula, Aeromonas, Magnetospirillum, Haloferax, Caryophanon, or Allochromatium. For example, the microorganism may be a Methylobacterium, e.g., Methylobacterium extorquens.

In some embodiments, the non-naturally occurring microorganism or the parent microorganism from which it is derived is genetically modified or artificially pre-selected to produce elevated levels of one or more carotenoid compound(s) relative to the corresponding unmodified or unselected microorganism. For example, the carotenoid compound(s) may be selected from, but are not limited to β-carotene, lycopene, rhodopsin, zeaxanthin, lutein, canthaxanthin, phoenicoxanthin, echinenone, cryptoxanthin, astaxanthin, adinoxanthin, 3-hydroxyechinenone, and/or sprilloxanthin.

In some embodiments, PHA is in one or more intracellular granule(s) in the non-naturally occurring microorganism.

In some embodiments, the non-naturally occurring microorganism includes one or more mutation(s) in one or more endogenous PHA biosynthesis gene(s), PHA degradation gene(s), and/or phasin gene(s), or external regulatory sequence(s) thereof, resulting in reduced or enhanced production of PHA, and/or PHA with an altered polymer length distribution.

In some embodiments, the mutation(s) include deletion or reduced expression of one or more PHA biosynthesis gene(s) (e.g., phaA, phaB, and/or phaC), or result in reduced enzymatic activity of one or more PHA biosynthetic enzyme(s) (e.g., gene product(s) of phaA, phaB, and/or phaC).

In some embodiments, the mutation(s) include enhanced expression of one or more PHA degradation gene(s) (e.g., phaY, phaZ, and/or hbd), or result in enhanced enzymatic activity of one or more PHA degradation enzyme(s) (e.g., gene products of phaY, phaZ, and/or hbd).

In some embodiments, the mutation(s) include deletion or reduced expression of one or more phasin gene(s) (e.g., Mext_2223, Mext_2560, and/or Mext_0493), or result in reduced binding affinity of one or more phasin(s) (e.g., gene products of Mext_2223, Mext_2560, and/or Mext_0493) for intracellular PHA granules.

In some embodiments, the non-naturally occurring microorganism includes one or more heterologous gene(s), resulting in reduced or enhanced production of PHA. For example, the non-naturally occurring microorganism may include one or more heterologous PHA degradation gene(s) (e.g., phaY and/or phaZ), resulting in reduced production of PHA or PHA with an altered polymer length distribution.

In another aspect, feed and nutritional supplement compositions are provided that include non-naturally occurring microorganisms (biomass) as described herein. In some embodiments, the composition may include PHA and protein in a weight ratio of about 1:1000 to about 3:1, or about 1:1000 to about 1:6. In some embodiments, the PHA product in the composition includes PHB.

In some embodiments, the feed or nutritional supplement composition includes a plurality of non-naturally occurring microorganisms as described herein, each including mutation(s) in one or more PHA biosynthesis gene(s) and/or mutation(s) in one or more phasin(s), wherein each of the plurality of non-naturally occurring microorganisms produces PHA (e.g., PHB) and protein at a different level, and wherein the combination of non-naturally occurring microorganisms provides PHA and protein in the composition at a weight ratio of about 1:1000 to about 3:1, or about 1:1000 to about 1:6.

In another aspect, a method is provided for producing biomass, including culturing a microorganism (e.g., a non-naturally occurring microorganism as described herein or a naturally occurring microorganism) that produces that produces PHA (e.g., PHB) in a culture medium under conditions suitable for growth of the microorganism, wherein the culture conditions result in biomass comprising PHA:protein in a weight ratio of about 1:1000 to about 3:1, or about 1:1000 to 1:6.

In some embodiments, the microorganism is of the genus Methylomonas, Methylobacter, Methylococcus, Methylosinus, Methylocyctis, Methylomicrobium, Methanomonas, Methylophilus, Methylobacillus, Methylobacterium, Hyphomicrobium, Xanthobacter, Bacillus, Paracoccus, Nocardia, Arthrobacter, Rhodopseudomonas, Pseudomonas, Candida, Hansenula, Pichia, Torulopsis, Vibrio, Escherichia, Alcaligenes, Ralstonia, Rhodobacter, Saccharomyces, Cupriavidus, Sinorhizobium, Mucor, Bradyrhizobium, Yarrowia, Azotobacter, Synechocystisor, Rhodotorula, Aeromonas, Magnetospirillum, Haloferax, Caryophanon, or Allochromatium. For example, the microorganism may be a Methylobacterium, e.g., Methylobacterium extorquens.

In some embodiments, the culture conditions include one or more alcohol(s) as a carbon source for producing said biomass, for example, but not limited to, methanol, ethanol, glycerol, or a combination thereof.

In some embodiments, the culture conditions include one or more alcohols(s) as a carbon source and additionally one or more organic acid(s), for example, but not limited to, formate, acetate, propionate, glycerate, malate, succinate, or a combination thereof.

In some embodiment, the culture conditions include aeration of the culture medium. For example, aeration of the medium may result in dissolved oxygen in the culture medium of about 5% to about 50%.

In some embodiments, the culture conditions include a temperature of about 20° C. to about 50° C.

In some embodiments, the culture conditions include removal of a portion of about 10% to about 90% of the culture medium when the culture reaches an optical density measured at 600 nm of about 50 to about 200, followed by replacement with an equivalent amount of fresh medium, thereby maintaining PHA production at a relatively constant level.

In some embodiments, the culture conditions include continuous removal of culture medium and microorganisms and continuous replenishment with fresh culture medium.

In some embodiments, the microorganism is genetically modified or artificially pre-selected to produce elevated levels of one or more carotenoid compound(s) relative to the corresponding unmodified or unselected microorganism. For example, the one or more carotenoid compound(s) may include, but are not limited to, β-carotene, lycopene, rhodopsin, zeaxanthin, lutein, canthaxanthin, phoenicoxanthin, echinenone, cryptoxanthin, astaxanthin, adinoxanthin, 3-hydroxyechinenone and/or sprilloxanthin. In some embodiments, the culture conditions for growth of the microorganism that has been genetically modified or artificially pre-selected to produce elevated levels of one or more carotenoid compound(s) includes one or more alcohol(s) as a carbon source, for example, but not limited to, methanol, ethanol, glycerol, or a combination thereof. In some embodiments, the culture conditions include one or more alcohols(s) as a carbon source and additionally one or more organic acid(s), for example, but not limited to, formate, acetate, propionate, glycerate, malate, succinate, or a combination thereof.

In some embodiments, PHA produced in the method is in one or more intracellular granule(s) in the microorganism.

In some embodiments, the microorganism is a non-naturally occurring microorganism that produces about 99.9% to about 1% less of a polyhydroxyalkanoate (PHA) product by weight and about 1% to about 250% more protein by weight than the parent microorganism from which the non-naturally occurring microorganism is derived.

In some embodiments of the method, the microorganism is a non-naturally occurring microorganism that includes mutation(s) in one or more endogenous PHA biosynthesis gene(s), PHA degradation gene(s), and/or phasin gene(s), resulting in reduced or enhanced production of PHA and/or PHA with an altered polymer length distribution.

In some embodiments, the non-naturally occurring microorganism produces PHA polymers that have an altered polymer size length distribution.

In some embodiments, the non-naturally occurring microorganism contains increased amounts of native or heterologous PHA degrading enzymes.

In some embodiments, the non-naturally occurring microorganism with increased production of native or heterologous PHA degrading enzymes is a component of a feed or nutritional supplement.

In some embodiments, the non-naturally occurring microorganism within a feed or nutritional supplement retains additional PHB degrading activity due to increased production of native or heterologous PHA degrading enzymes.

In another aspect, a feed or nutritional supplement composition is provided that includes biomass produced in a method as described herein.

In another aspect, a method is provided for improving survivability of a livestock, seafood, or aquaculture animal, including feeding the animal a feed composition that includes biomass produced in a method as described herein, and wherein the survivability is increased by at least about 1% in comparison to a feed composition that includes no PHA. In some embodiments, the PHA is PHB. In some embodiments, the feed composition includes a plurality of microorganisms, wherein each of the plurality of microorganisms produces PHA and protein at a different level, and wherein the combination of microorganisms provides PHA and protein in the composition at a weight ratio of about 1:1000 to about 3:1, or about 1:1000 to about 1:6.

### BRIEF DESCRIPTION OF THE DRAWINGS

FIG. 1 shows a schematic diagram of an embodiment of a PHA biosynthesis and degradation pathway.

FIGS. 2A-2B show results of phasin deletion on PHB production, as described in Example 1.

FIG. 3 shows the results of aeration level on PHB production, as described in Example 2.

FIGS. 4A-4B show the results of temperature on PHB production, as described in Example 2.

FIG. 5 shows the results of the fill and draw experiment described in Example 2.

FIGS. 6A-6B shows correlation of PHB levels with protein content of cells, as described in Example 2.

FIG. 7 shows survivability of shrimp on diets with and without PHB, as described in Example 3.

FIG. 8 shows the results of methanol-ethanol carbon source on PHB production levels, as described in Example 2.

FIG. 9 shows the results in increasing ethanol concentration on PHB production, as described in Example 4.

FIGS. 10A-10D show the Gel Permeation Chromatography (GPC) trace from the refractive index detector (RFID) of PHB extracted from cells as described in Example 5.

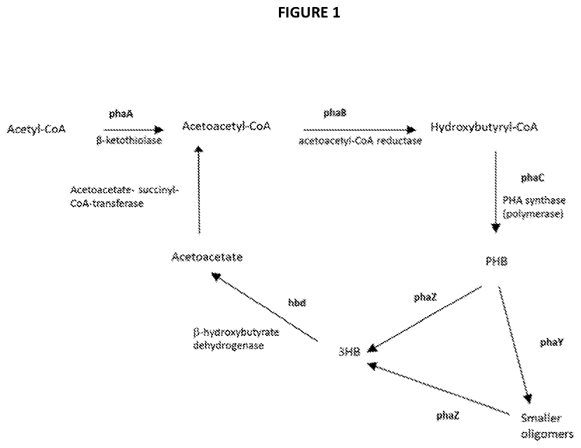

### DETAILED DESCRIPTION

The invention provides microorganisms and methods of culturing microorganisms to produce biomass with PHA (e.g., PHB) and protein levels that are advantageous for inclusion in feed and nutritional compositions. By lowering PHA production, through genetics or through fermentation processes, protein content in the biomass may be enriched from about 40% to about 70% or higher. Additionally, average PHA polymer length can be decreased to increase bioavailability.

### Definitions

Unless defined otherwise herein, all technical and scientific terms used herein have the same meaning as commonly understood by one of ordinary skill in the art to which this invention belongs. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the present invention.

The practice of the present invention will employ, unless otherwise indicated, conventional techniques of molecular biology (including recombinant techniques), microbiology, cell biology, and biochemistry, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polvmerase Chain Reaction (Mullis et al., eds., 1994); and Gene Transfer and Expression: A Laboratory Manual (Kriegler, 1990).

Numeric ranges provided herein are inclusive of the numbers defining the range.

Unless otherwise indicated, nucleic acids are written left to right in 5′ to 3′ orientation; amino acid sequences are written left to right in amino to carboxy orientation, respectively.

“A,” “an” and “the” include plural references unless the context clearly dictates otherwise.

As used herein, the term “polynucleotide” refers to a polymeric form of nucleotides of any length and any three-dimensional structure and single- or multi-stranded (e.g., single-stranded, double-stranded, triple-helical, etc.), which contain deoxyribonucleotides, ribonucleotides, and/or analogs or modified forms of deoxyribonucleotides or ribonucleotides, including modified nucleotides or bases or their analogs. Because the genetic code is degenerate, more than one codon may be used to encode a particular amino acid, and the present invention encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, etc.). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. The terms “polynucleotide,” “nucleic acid,” and “oligonucleotide” are used herein interchangeably. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. Polynucleotides may be linear or circular or comprise a combination of linear and circular portions.

As used herein, “polypeptide” refers to a composition comprised of amino acids and recognized as a protein by those of skill in the art. The conventional one-letter or three-letter code for amino acid residues is used herein. The terms “polypeptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, a “vector” refers to a polynucleotide sequence designed to introduce nucleic acids into one or more cell types. Vectors include cloning vectors, expression vectors, shuttle vectors, plasmids, phage particles, cassettes and the like.

As used herein, the term “expression” refers to the process by which a polypeptide is produced based on the nucleic acid sequence of a gene. The process includes both transcription and translation.

As used herein, “expression vector” refers to a DNA construct containing a DNA coding sequence (e.g., gene sequence) that is operably linked to one or more suitable control sequence(s) capable of effecting expression of the coding sequence in a host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, or simply a potential genomic insert. Once transformed into a suitable host, the vector may replicate and function independently of the host genome, or may, in some instances, integrate into the genome itself. The plasmid is the most commonly used form of expression vector. However, the invention is intended to include such other forms of expression vectors that serve equivalent functions and which are, or become, known in the art.

A “promoter” refers to a regulatory sequence that is involved in binding RNA polymerase to initiate transcription of a gene. A promoter may be an inducible promoter or a constitutive promoter. An “inducible promoter” is a promoter that is active under environmental or developmental regulatory conditions.

The term “operably linked” refers to a juxtaposition or arrangement of specified elements that allows them to perform in concert to bring about an effect. For example, a promoter is operably linked to a coding sequence if it controls the transcription of the coding sequence.

“Under transcriptional control” is a term well understood in the art that indicates that transcription of a polynucleotide sequence depends on its being operably linked to an element which contributes to the initiation of, or promotes transcription.

“Under translational control” is a term well understood in the art that indicates a regulatory process which occurs after mRNA has been formed.

A “gene” refers to a DNA segment that is involved in producing a polypeptide and includes regions preceding and following the coding regions as well as intervening sequences (introns) between individual coding segments (exons).

As used herein, the term “host cell” refers to a cell or cell line into which a recombinant expression vector for production of a polypeptide may be transfected for expression of the polypeptide. Host cells include progeny of a single host cell, and the progeny may not necessarily be completely identical (in morphology or in total genomic DNA complement) to the original parent cell due to natural, accidental, or deliberate mutation. A host cell includes cells transfected or transformed in vivo with an expression vector.

The term “recombinant,” refers to genetic material (i.e., nucleic acids, the polypeptides they encode, and vectors and cells comprising such polynucleotides) that has been modified to alter its sequence or expression characteristics, such as by mutating the coding sequence to produce an altered polypeptide, fusing the coding sequence to that of another gene, placing a gene under the control of a different promoter, expressing a gene in a heterologous organism, expressing a gene at a decreased or elevated levels, expressing a gene conditionally or constitutively in manner different from its natural expression profile, and the like. Generally recombinant nucleic acids, polypeptides, and cells based thereon, have been manipulated by man such that they are not identical to related nucleic acids, polypeptides, and cells found in nature.

A “signal sequence” refers to a sequence of amino acids bound to the N-terminal portion of a protein which facilitates the secretion of the mature form of the protein from the cell. The mature form of the extracellular protein lacks the signal sequence which is cleaved off during the secretion process.

The term “selective marker” or “selectable marker” refers to a gene capable of expression in a host cell that allows for ease of selection of those hosts containing an introduced nucleic acid or vector. Examples of selectable markers include but are not limited to antimicrobial substances (e.g., hygromycin, bleomycin, kanamycin or chloramphenicol) and/or genes that confer a metabolic advantage, such as a nutritional advantage, on the host cell.

The term “derived from” encompasses the terms “originated from,” “obtained from,” “obtainable from,” “isolated from,” and “created from,” and generally indicates that one specified material finds its origin in another specified material or has features that can be described with reference to another specified material.

The term “culturing” refers to growing a population of cells, e.g., microbial cells, under suitable conditions for growth, in a liquid or solid medium.

The term “heterologous” or “exogenous,” with reference to a polynucleotide or protein, refers to a polynucleotide or protein that does not naturally occur in a specified cell, e.g., a host cell. It is intended that the term encompass proteins that are encoded by naturally occurring genes, mutated genes, and/or synthetic genes. In contrast, the term “homologous,” with reference to a polynucleotide or protein, refers to a polynucleotide or protein that occurs naturally in the cell.

The term “introduced,” in the context of inserting a nucleic acid sequence into a cell, includes “transfection,” “transformation,” or “transduction” and refers to the incorporation of a nucleic acid sequence into a eukaryotic or prokaryotic cell wherein the nucleic acid sequence may be incorporated into the genome of the cell (e.g., chromosome, plasmid, plastid, or mitochondrial DNA), converted into an autonomous replicon, or transiently expressed.

“Transfection” or “transformation” refers to the insertion of an exogenous polynucleotide into a host cell. The exogenous polynucleotide may be maintained as a non-integrated vector, for example, a plasmid, or alternatively, may be integrated into the host cell genome. The term “transfecting” or “transfection” is intended to encompass all conventional techniques for introducing nucleic acid into host cells. Examples of transfection techniques include, but are not limited to, calcium phosphate precipitation, DEAE-dextran-mediated transfection, lipofection, electroporation, and microinjection.

As used herein, the terms “transformed,” “stably transformed,” and “transgenic” refer to a cell that has a non-native (e.g., heterologous) nucleic acid sequence integrated into its genome or as an episomal plasmid that is maintained through multiple generations.

The terms “recovered,” “isolated,” “purified,” and “separated” as used herein refer to a material (e.g., a protein, nucleic acid, or cell) that is removed from at least one component with which it is naturally associated. For example, these terms may refer to a material which is substantially or essentially free from components which normally accompany it as found in its native state, such as, for example, an intact biological system.

A “signal sequence” (also termed “presequence,” “signal peptide,” “leader sequence,” or “leader peptide”) refers to a sequence of amino acids at the amino terminus of a nascent polypeptide that targets the polypeptide to the secretory pathway and is cleaved from the nascent polypeptide once it is translocated in the endoplasmic reticulum membrane.

Related (and derivative) proteins encompass “variant” proteins. Variant proteins differ from a parent protein and/or from one another by a small number of amino acid residues. In some embodiments, the number of different amino acid residues is any of about 1, 2, 3, 4, 5, 10, 20, 25, 30, 35, 40, 45, or 50. In some embodiments, variants differ by about 1 to about 10 amino acids. Alternatively or additionally, variants may have a specified degree of sequence identity with a reference protein or nucleic acid, e.g., as determined using a sequence alignment tool, such as BLAST, ALIGN, and CLUSTAL (see, infra). For example, variant proteins or nucleic acid may have at least about 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% amino acid sequence identity with a reference sequence.

As used herein, the term “analogous sequence” refers to a polypeptide sequence within a protein that provides a similar function, tertiary structure, and/or conserved residues with respect to a reference protein. For example, in epitope regions that contain an alpha helix or a beta sheet structure, replacement amino acid(s) in an analogous sequence maintain the same structural element. In some embodiments, analogous sequences are provided that result in a variant enzyme exhibiting a similar or improved function with respect to the parent protein from which the variant is derived.

As used herein, “homologous protein” refers to a protein that has similar function and/or structure as a reference protein. Homologs may be from evolutionarily related or unrelated species. In some embodiments, a homolog has a quaternary, tertiary and/or primary structure similar to that of a reference protein, thereby potentially allowing for replacement of a segment or fragment in the reference protein with an analogous segment or fragment from the homolog, with reduced disruptiveness of structure and/or function of the reference protein in comparison with replacement of the segment or fragment with a sequence from a non-homologous protein.

As used herein, “wild-type,” “native,” and “naturally-occurring” proteins are those found in nature. The terms “wild-type sequence” refers to an amino acid or nucleic acid sequence that is found in nature or naturally occurring. In some embodiments, a wild-type sequence is the starting point of a protein engineering project, for example, production of variant proteins.

The phrases “substantially similar” and “substantially identical” in the context of at least two nucleic acids or polypeptides typically means that a polynucleotide, polypeptide, or region or domain of a polypeptide that comprises a sequence that has at least about 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% sequence identity, in comparison with a reference (e.g., wild-type) polynucleotide, polypeptide, or region or domain of a polypeptide. A region or domain of a polypeptide may contain, for example, at least about 20, 50, 100, or 200 amino acids within a longer polypeptide sequence. Sequence identity may be determined using known programs such as BLAST, ALIGN, and CLUSTAL using standard parameters. (See, e.g., Altshul, et al. (1990) J. Mol. Biol. 215:403-410; Henikoff, et al. (1989) Proc. Natl. Acad. Sci. 89:10915; Karin, et al. (1993) Proc. Natl. Acad. Sci. 90:5873; and Higgins, et al. (1988) Gene 73:237). Software for performing BLAST analyses is publicly available through the National Center for Biotechnology Information. Also, databases may be searched using FASTA (Person, et al. (1988) Proc. Natl. Acad. Sci. 85:2444-2448.) In some embodiments, substantially identical polypeptides differ only by one or more conservative amino acid substitutions. In some embodiments, substantially identical polypeptides are immunologically cross-reactive. In some embodiments, substantially identical nucleic acid molecules hybridize to each other under stringent conditions (e.g., within a range of medium to high stringency).

The term “carotenoid” is understood in the art to refer to a structurally diverse class of pigments derived from isoprenoid pathway intermediates. The commitment step in carotenoid biosynthesis is the formation of phytoene from geranylgeranyl pyrophosphate. Carotenoids can be acyclic or cyclic, and may or may not contain oxygen, so that the term carotenoids include both carotenes and xanthophylls. In general, carotenoids are hydrocarbon compounds having a conjugated polyene carbon skeleton formally derived from the five-carbon compound IPP, including triterpenes (C₃₀ diapocarotenoids) and tetraterpenes (C₄₀ carotenoids) as well as their oxygenated derivatives and other compounds that are, for example, C₃₅, C₅₀, C₆₀, C₇₀, C₈₀ in length or other lengths. Many carotenoids have strong light absorbing properties and may range in length in excess of C₂₀₀-C₃₀ diapocarotenoids typically consist of six isoprenoid units joined in such a manner that the arrangement of isoprenoid units is reversed at the center of the molecule so that the two central methyl groups are in a 1,6-positional relationship and the remaining non-terminal methyl groups are in a 1,5-positional relationship. Such C₃₀ carotenoids may be formally derived from the acyclic C₃₀H₄₂ structure, having a long central chain of conjugated double bonds, by: (i) hydrogenation (ii) dehydrogenation, (iii) cyclization, (iv) oxidation, (v) esterification/glycosylation, or any combination of these processes. C₄₀ carotenoids typically consist of eight isoprenoid units joined in such a manner that the arrangement of isoprenoid units is reversed at the center of the molecule so that the two central methyl groups are in a 1,6-positional relationship and the remaining non-terminal methyl groups are in a 1,5-positional relationship. Such C₄₀ carotenoids may be formally derived from the acyclic C₄₀H₅₆ structure, having a long central chain of conjugated double bonds, by (i) hydrogenation, (ii) dehydrogenation, (iii) cyclization, (iv) oxidation, (v) esterification/glycosylation, or any combination of these processes. The class of C₄₀ carotenoids also includes certain compounds that arise from rearrangements of the carbon skeleton, or by the (formal) removal of part of this structure. More than 600 different carotenoids have been identified in nature. Carotenoids include but are not limited to: antheraxanthin, adonirubin, adonixanthin, astaxanthin, canthaxanthin, capsorubrin, β-cryptoxanthin, α-carotene, β-carotene, β,ψ-carotene, δ-carotene, ε-carotene, echinenone, 3-hydroxyechinenone, 3′-hydroxyechinenone, γ-carotene, ψ-carotene, 4-keto-Y-carotene, ζ-carotene, a-cryptoxanthin, deoxyflexixanthin, diatoxanthin, 7,8-didehydroastaxanthin, didehydrolycopene, fucoxanthin, fucoxanthinol, isorenieratene, β-isorenieratene, lactucaxanthin, lutein, lycopene, myxobactone, neoxanthin, neurosporene, hydroxyneurosporene, peridinin, phytoene, rhodopin, rhodopin glucoside, 4-keto-rubixanthin, siphonaxanthin, spheroidene, spheroidenone, spirilloxanthin, torulene, 4-keto-torulene, 3-hydroxy-4-keto-torulene, uriolide, uriolide acetate, violaxanthin, zeaxanthin-β-diglucoside, zeaxanthin, and C30 carotenoids. Additionally, carotenoid compounds include derivatives of these molecules, which may include hydroxy-, methoxy-, oxo-, epoxy-, carboxy-, or aldehydic functional groups. Further, included carotenoid compounds include ester (e.g., glycoside ester, fatty acid ester) and sulfate derivatives (e.g., esterified xanthophylls).

The “isoprenoid pathway” is understood in the art to refer to a metabolic pathway that either produces or utilizes the five-carbon metabolite isopentyl pyrophosphate (IPP). As discussed herein, two different pathways can produce the common isoprenoid precursor IPP— the “mevalonate pathway” and the “non-mevalonate pathway.” The term “isoprenoid pathway” is sufficiently general to encompass both of these types of pathway. Biosynthesis of isoprenoids from IPP occurs by polymerization of several five-carbon isoprene subunits. Isoprenoid metabolites derived from IPP vary greatly in chemical structure, including both cyclic and acyclic molecules. Isoprenoid metabolites include, but are not limited to, monoterpenes, sesquiterpenes, diterpenes, sterols, and polyprenols such as carotenoids.

The term “isoprenoid compound” refers to any compound which is derived via the pathway beginning with isopentenyl pyrophosphate (IPP) and formed by the head-to-tail condensation of isoprene units which may be of 5, 10, 15, 20, 30 or 40 carbons in length. There term “isoprenoid pigment” refers to a class of isoprenoid compounds which typically have strong light absorbing properties.

The term “feed premix” refers to the crude mixture of aquaculture feed or animal/pet food components prior to processing, optionally at high temperature, into an aquaculture feed or animal or pet food composition that is in the form of pellets or flakes.

An aquaculture feed composition is used in the production of an “aquaculture product,” wherein the product is a harvestable aquacultured species (e.g., finfish, crustaceans), which is often sold for human consumption. For example, salmon are intensively produced in aquaculture and thus are aquaculture products. Aquaculture compositions may also be used as feed for aquaculture feed organisms such as small fish like krill, rotifers, and the like, that are food sources for larger aquaculture organisms such as carnivorous fish. In addition, aquaculture compositions described herein can be used as feed for ornamental fish, shrimp, hobbyist aquaculture, and the like, that are not intended as food for other organisms.

The term “aquaculture meat product” refers to food products intended for human consumption comprising at least a portion of meat from an aquaculture product as defined above. An aquaculture meat product may be, for example, a whole fish or a filet cut from a fish, each of which may be consumed as food. In some embodiments, such a product can be referred to as a fish or seafood product.

The term “biomass” refers to microbial cellular material. Biomass may be produced naturally, or may be produced from the fermentation of a native host or a recombinant production host. The biomass may be in the form of whole cells, whole cell lysates, homogenized cells, partially hydrolyzed cellular material, and/or partially purified cellular material (e.g., microbially produced oil).

The term “processed biomass” refers to biomass that has been subjected to additional processing such as drying, pasteurization, disruption, etc., each of which is discussed in greater detail below.

The term “C-1 carbon substrate” refers to any carbon-containing molecule that lacks a carbon-carbon bond. Examples are methane, methanol, formaldehyde, formic acid, formate, methylated amines (e.g., mono-, di-, and tri-methyl amine), methylated thiols, and carbon dioxide.

The term “C1 metabolizer” refers to a microorganism that has the ability to use a single carbon substrate as a sole source of energy and biomass. C1 metabolizers will typically be methylotrophs and/or methanotrophs capable of growth.

The term “methylotroph” means an organism capable of oxidizing organic compounds which do not contain carbon-carbon bonds. Where the methylotroph is able to oxidize CH₄, the methylotroph is also a methanotroph.

The term “methanotroph” means a prokaryote capable of utilizing methane as a substrate. Complete oxidation of methane to carbon dioxide occurs by aerobic degradation pathways. Typical examples of methanotrophs useful in the present invention include but are not limited to the genera Methylomonas, Methylobacter, Methylococcus, and Methylosinus.

The term “high growth methanotrophic bacterial strain” refers to a bacterium capable of growth using methane as its sole carbon and energy source.

The term “phasin” refers to a protein that enhances PHA production by binding to granules and increasing the surface/volume ratio of the granules, or a protein that activates the rate of PHA synthesis by interacting directly with PHA synthase or promotes PHA synthesis indirectly by preventing growth defects associated with the binding of other cellular proteins to PHA granules. (See. e.g., York, et al. (2001)J Bacteriol 183 (7):2394-97)

“Survivability” refers to resulting in or promoting survival. For example, feed products or supplements that increase survivability will increase the number of harvested fish, invertebrates, or other animals relative to another feed or nutritional supplement.

The term “dissolved oxygen” (“DO”) refers to the amount of free oxygen dissolved in water which is readily available to respiring organisms. “% dissolved oxygen” (“% DO) refers to oxygen as a percentage of air saturation, and is dependent, e.g., on temperature, pressure, and salinity of the medium in which it is dissolved. Measured % DO=O₂ mg/L/(DO value at temperature and salinity). % DO is a relative term based on the maximum amount of oxygen at a given temperature. For example, at higher temperatures, the actual amount of oxygen dissolved for, e.g., 50% DO, is less.

“Continuous” fermentation or “fed-batch” refers to a steady-state fermentation system in which substrate is continuously added to a fermenter while products and residues are removed at a steady rate.

“Semi-continuous” or “fill and draw” fermentation refers to a fermentation process in which cells are maintained in an actively dividing state in the culture by periodically draining off the medium and replenishing it with fresh medium.

“Gel Permeation Chromatography” or “Size Exclusion Chromatography” (SEC) refers to a chromatographic process by which molecules are separated based on size. Larger molecules are eluted more quickly than smaller molecules because they are excluded and do not permeate the pores in the chromatographic matrix. By using a standard comprised of multiple components of known molecular weights, the average molecular weight and the relative distribution of molecules in a sample can be ascertained. There are several molecular mass determinations for disperse polymer samples, including the number average molecular weight (Mn), weight average molecular weight (Mw), peak molecular weight (Mp), and Z-average molecular weight (Mz). Additionally, the polydispersity index (PD) is used as a measure of the broadness of a molecular weight distribution of a polymer. Polymers with smaller PD have molecular weights that are closer to the mean. PD is equal to Mw divided by Mn.

“Altered polymer size length distribution” refers to polymers with an average molecular mass (Mw, Mn, Mp, Mz) or distribution (PD) that is different than in a comparison strain, e.g., a wild type strain. For example, the introduction of enzymes that cleave a PHA polymer into smaller oligomers would decrease the Mw, Mn, Mp, and Mz, in comparison to the original parent strain. Unless cleavage was complete, the resulting smaller oligomers would also increase the polydispersity index.

“Polymers that have reduced molecular weight on average” refers to polymers that have reduced Mw, Mn, Mp, or Mz as measured by GPC using a molecular weight size standard as is commonly determined in the art.

“Digestibility” refers to the ability of a polymer to be degraded by enzymatic, thermal, or chemical means into smaller oligomers or individual polymer subunits.

### Microorganisms

### Non-Naturally Occurring Microorganisms

In some embodiments, non-naturally occurring microorganisms are provided that produce PHA (e.g., PHB) at either reduced or elevated levels in comparison to the parent microorganism from which they are derived. The parent microorganism may be either a wild type microorganism (i.e., found in nature) or may be a non-naturally occurring mutant or a genetically engineered (e.g., recombinant) microorganism.

In some embodiments, a non-naturally occurring microorganism herein may produce about 1% to about 99.9% less PHA (e.g., PHB) and about 1% to about 250% more protein than the parent microorganism from which it is derived. For example, a non-naturally occurring microorganism herein may produce any of about 1% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 95%, or about 95% to about 99.5 less PHA (e.g., PHB), and any of about 1% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 110%, about 110% to about 120%, about 120% to about 130%, about 130% to about 140%, about 140% to about 150%, about 150% to about 160%, about 160% to about 170%, about 170% to about 180%, about 180% to about 190%, about 190% to about 200%, about 200% to about 210%, about 210% to about 220%, about 220% to about 230%, about 230% to about 240%, or about 240% to about 250% more protein than the parent microorganism from which it is derived.

In some embodiments, a non-naturally occurring microorganism herein may produce about 100% to about 300% more PHA (e.g., PHB) and about 1% to about 250% more protein than the parent microorganism from which it is derived. For example, a non-naturally occurring microorganism herein may produce any of about 100% to about 125%, about 125% to about 150%, about 150% to about 175%, about 175% to about 200%, about 200% to about 225%, about 225% to about 250%, or about 250% to about 300% more PHA (e.g., PHB), and any of about 1% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 110%, about 110% to about 120%, about 120% to about 130%, about 130% to about 140%, about 140% to about 150%, about 150% to about 160%, about 160% to about 170%, about 170% to about 180%, about 180% to about 190%, about 190% to about 200%, about 200% to about 210%, about 210% to about 220%, about 220% to about 230%, about 230% to about 240%, or about 240% to about 250% more protein than the parent microorganism from which it is derived.

Non-naturally occurring microorganisms herein include, e.g., bacteria, yeast, Archaea, that produce PHA when cultured under conditions suitable for microbial growth and PHA (e.g., PHB) production. In some embodiments, the microorganisms produce about 0.1% to about 50% PHA by weight, based on dry cell weight (dcw) and about 35% to about 70% or more, about 60% to about 70%, or about 65% protein per dcw. For example, a non-naturally occurring microorganism herein may produce any of about 0.1% to about 0.5%, about 0.5% to about 1%, about 1% to about 5%, about 5% to about 10%, about 10% to about 15%, about 15% to about 20%/a, about 20% to about 25%, about 25% to about 30%, about 30% to about 35%, about 35% to about 40%, about 40% to about 45%, or about 45% to about 50% PHA (e.g., PHB), and any of about 35% to about 40%, about 40% to about 45%, about 45% to about 50%, about 50% to about 55%, about 55% to about 60%, about 60% to about 65%, about 65% to about 70%, or greater than about 70% protein per dcw.

In some embodiments, the non-naturally occurring microorganisms produce PHA (e.g., PHB) and protein at a PHA:protein weight ratio that is about 1:1000 to about 3:1, about 1:1000 to about 1:6, about 1:1 to about 2:1, about 1:1 to about 3:1, or about 2:1 to about 3:1. In some embodiments, the PHA:protein ratio is about 1:1000 to about 1:500, about 1:500 to about 1:100, about 1:100 to about 1:50, about 1:50 to about 1:10, about 1:10 to about 1:6, about 1:6 to about 1:2, or about 1:2 to about 1:1. In some embodiments, at any of the ratios of PHA:protein described herein, the microorganism may produce about 35% to about 70% or more, about 60% to about 70%, or about 65% protein, or any of about 35% to about 40%, about 40% to about 45%, about 45% to about 50%, about 50% to about 55%, about 55% to about 60%, about 60% to about 65%, about 65% to about 70%, or greater than about 70% protein per dcw.

In some embodiments, a non-naturally occurring microorganism herein may produce PHA polymers with reduced average molecular weight (Mw, Mn, Mp, or Mz), increased polydispersity, or increased digestibility.

The PHA (e.g., PHB) produced by a non-naturally occurring microorganism as described herein may be contained in one or more intracellular granule(s) in the cell.

Non-limiting examples of genera from which the non-naturally occurring microorganism may be derived include Methylomonas, Methylobacter, Methylococcus, Methylosinus, Methylocyctis, Methylomicrobium, Methanomonas, Methylophilus, Methylobacillus, Methylobacterium, Hyphomicrobium, Xanthobacter, Bacillus, Paracoccus, Nocardia, Arthrobacter, Rhodopseudomonas, Pseudomonas, Candida, Hansenula, Pichia, Torulopsis, Vibrio, Escherichia, Alcaligenes, Ralstonia, Rhodobacter, Saccharomyces, Cupriavidus, Sinorhizobium, Mucor, Bradyrhizobium, Yarrowia, Azotobacter, Synechocystis, Rhodotorula, Aeromonas, Magnetospirillum, Haloferax, Caryophanon, and Allochromatium.

Non-limiting examples of microbial species from which the non-naturally occurring microorganism may be derived include Methylobacterium extorquens (e.g., strains AM1, DM4, CM4, PA1, DSMZ 1340), Methylobacterium populi (BJ001), Methylobacterium radiotolerans, Methylobacterium nodulans, Methylobacterium sp 4-46, and other Methylobacterium species.

In some embodiments, the non-naturally occurring microorganism is a methylotrophic bacterium.

In some embodiments, the non-naturally occurring microorganism has been modified to utilize one or more alcohol(s) as a carbon source, including but not limited to methanol, ethanol, propanol, and/or glycerol.

In some embodiments, the non-naturally occurring microorganism or the parent cell from which the non-naturally occurring microorganism is derived is genetically modified or artificially pre-selected to produce elevated levels of one or more carotenoid compound(s) relative to the corresponding unmodified or unselected microorganism. The one or more carotenoid compound(s) may include, but are not limited to, δ-carotene, lycopene, zeaxanthin, rhodopsin, zeaxanthin, lutein, canthaxanthin, phoenicoxanthin, echinenone, cryptoxanthin, astaxanthin, adinoxanthin, 3-hydroxyechinenone, and/or sprilloxanthin. Non-limiting examples of host cells that produce elevated levels of one or more carotenoid compound(s) and methods for producing such microorganisms are provided in WO2015/021352 A2.

In some embodiments, the parent microorganism from which a non-naturally occurring microorganism as described herein is derived contains deletions in the genes celA and/or carotenoid genes (crtC, crtD, and crtF).

A non-naturally occurring microorganism herein may include one or more mutation(s), for example, mutation(s) in one or more PHA biosynthesis gene(s) and/or one or more phasin(s).

In some embodiments, the microorganism may include mutation(s) in one or more endogenous PHA biosynthesis gene(s), such as, but not limited to, phaA, phaB, hbd, phaY, phaC, and/or phaZ, or their external regulatory sequences (i.e., promoter sequences). The mutation(s) may include deletion of the one or more PHA biosynthesis gene(s), reduced expression of the one or more PHA biosynthesis gene(s) (e.g., due to alteration of regulatory sequence(s)), or reduced enzymatic activity of the enzyme(s) encoded by the biosynthesis gene(s), resulting in reduced production of PHA (e.g., PHB). In various embodiments, PHA (e.g., PHB) is decreased by decreasing PHA biosynthesis enzyme activity, by deletion or modification of gene(s) that decrease(s) transcription, translation, or transcript stability of PHA biosynthesis enzyme(s), or by increasing (or introducing) transcription, translation, or transcript stability of PHA degrading enzyme(s).

In some embodiments, the microorganism may include mutation(s) that result in increased PHA (e.g., PHB) production. For example, regulatory sequence(s) of one or more PHA biosynthesis genes may be modified. In some embodiments, mutation(s) result in increased expression, or increased transcription, translation, or transcript stability of PHA biosynthesis enzyme(s), or decreased transcription, translation, transcript stability, or activity of PHA degrading enzyme(s). In some embodiments, mutation(s) in the coding sequence(s) result in increased activity of one or more PHA biosynthesis enzyme(s) or decreased activity in one or more PHA degradation enzyme(s). In some embodiments, exogenous PHA biosynthesis gene(s) may be added to the microorganism, either to introduce PHA biosynthesis activity that the organism does not possess or to increase copy number of endogenous PHA biosynthesis gene(s).

In some embodiments, the microorganism includes a mutation in the phaA polynucleotide sequence or in a regulatory sequence for expression of the polynucleotide sequence depicted in SEQ ID NO:1 or a polynucleotide having at least about 30, 35, 40, 45, 50, 55, 60, 65, 70, 75, 80, 85, 90, 95, 98, or 99% sequence identity to SEQ ID NO:1, for example, a deletion of at least a portion of the polynucleotide sequence, reduced expression of the polynucleotide sequence, and/or reduced enzymatic activity of the 3-ketothiolase enzyme encoded by the polynucleotide. In some embodiments, the microorganism includes a mutation in a polynucleotide that encodes a 3-ketothiolase amino acid sequence, for example, the amino acid sequence depicted in SEQ ID NO:2 or an amino acid sequence having at least about 30, 35, 40, 45, 50, 55, 60, 65, 70, 75, 80, 85, 90, 95, 98, or 99% sequence identity to SEQ ID NO:2 and retaining 3-ketothiolase enzyme activity.

In some embodiments, the microorganism includes a mutation in the phaB polynucleotide sequence or in a regulatory sequence for expression of the polynucleotide sequence depicted in SEQ ID NO:3 or a polynucleotide having at least about 30, 35, 40, 45, 50, 55, 60, 65, 70, 75, 80, 85, 90, 95, 98, or 99% sequence identity to SEQ ID NO:3, for example, a deletion of at least a portion of the polynucleotide sequence, reduced expression of the polynucleotide sequence, and/or reduced enzymatic activity of the acetoacetyl-CoA reductase enzyme encoded by the polynucleotide. In some embodiments, the microorganism includes a mutation in a polynucleotide that encodes an acetoacetyl-CoA reductase amino acid sequence, for example, the amino acid sequence depicted in SEQ ID NO:4 or an amino acid sequence having at least about 30, 35, 40, 45, 50, 55, 60, 65, 70, 75, 80, 85, 90, 95, 98, or 99% sequence identity to SEQ ID NO:4 and retaining acetoacetyl-CoA reductase enzyme activity.

In some embodiments, the microorganism includes a mutation in the phaC polynucleotide sequence or in a regulatory sequence for expression of the polynucleotide sequence depicted in SEQ ID NO:5 or a polynucleotide having at least about 30, 35, 40, 45, 50, 55, 60, 65, 70, 75, 80, 85, 90, 95, 98, or 99% sequence identity to SEQ ID NO:5, for example, a deletion of at least a portion of the polynucleotide sequence, reduced expression of the polynucleotide sequence, and/or reduced enzymatic activity of the PHA synthase (polymerase) enzyme encoded by the polynucleotide. In some embodiments, the microorganism includes a mutation in a polynucleotide that encodes a PHA synthase (polymerase) amino acid sequence, for example, the amino acid sequence depicted in SEQ ID NO:6 or an amino acid sequence having at least about 30, 35, 40, 45, 50, 55, 60, 65, 70, 75, 80, 85, 90, 95, 98, or 99% sequence identity to SEQ ID NO:6 and retaining PHA synthase (polymerase) enzyme activity.

In some embodiments, the microorganism may include mutation(s) in one or more phasin gene(s), such as, but not limited to, Mext_2223, Mext_2560, and/or Mext_0493. The mutation(s) may include deletion or reduced expression of the one or more phasin gene(s), or reduced binding affinity of the phasin for intracellular PHA granules, resulting in reduced production of PHA (e.g., PHB), more digestible PHA, or PHA with an altered molecular weight distribution. In some embodiments, the microorganism may include a modification to increase expression of one or more phasin(s) (e.g., by increasing promoter strength or gene copy number), thereby producing smaller, more digestible PHA granules or PHA with an altered molecular weight distribution.

In some embodiments, the microorganism includes a mutation in the Mext_0493 polynucleotide sequence or in a regulatory sequence for expression of the polynucleotide sequence depicted in SEQ ID NO:7 or a polynucleotide having at least about 30, 35, 40, 45, 50, 55, 60, 65, 70, 75, 80, 85, 90, 95, 98, or 99% sequence identity to SEQ ID NO:7, for example, a deletion of at least a portion of the polynucleotide sequence, reduced expression of the polynucleotide sequence, and/or reduced binding affinity of the phasin encoded by the polynucleotide for intracellular PHA granules. In some embodiments, the microorganism includes a mutation in a polynucleotide that encodes the Mext_0493 amino acid sequence depicted in SEQ ID NO:8 or that encodes an amino acid sequence having at least about 30, 35, 40, 45, 50, 55, 60, 65, 70, 75, 80, 85, 90, 95, 98, or 99% sequence identity to SEQ ID NO:8 and retaining binding affinity for intracellular PHA granules.

In some embodiments, the microorganism includes a mutation in the Mext_2223 polynucleotide sequence or in a regulatory sequence for expression of the polynucleotide sequence depicted in SEQ ID NO:9 or a polynucleotide having at least about 30, 35, 40, 45, 50, 55, 60, 65, 70, 75, 80, 85, 90, 95, 98, or 99% sequence identity to SEQ ID NO:9, for example, a deletion of at least a portion of the polynucleotide sequence, reduced expression of the polynucleotide sequence, and/or reduced binding affinity of the phasin encoded by the polynucleotide for intracellular PHA granules. In some embodiments, the microorganism includes a mutation in a polynucleotide that encodes the Mext_2223 amino acid sequence depicted in SEQ ID NO: 10 or that encodes an amino acid sequence having at least about 30, 35, 40, 45, 50, 55, 60, 65, 70, 75, 80, 85, 90, 95, 98, or 99% sequence identity to SEQ ID NO:10 and retaining binding affinity for intracellular PHA granules.

In some embodiments, the microorganism includes a mutation in the Mext_2560 polynucleotide sequence or in a regulatory sequence for expression of the polynucleotide sequence depicted in SEQ ID NO:11 or a polynucleotide having at least about 30, 35, 40, 45, 50, 55, 60, 65, 70, 75, 80, 85, 90, 95, 98, or 99% sequence identity to SEQ ID NO: 11, for example, a deletion of at least a portion of the polynucleotide sequence, reduced expression of the polynucleotide sequence, and/or reduced binding affinity of the phasin encoded by the polynucleotide for intracellular PHA granules. In some embodiments, the microorganism includes a mutation in a polynucleotide that encodes the Mext_2560 amino acid sequence depicted in SEQ ID NO: 12 or that encodes an amino acid sequence having at least about 30, 35, 40, 45, 50, 55, 60, 65, 70, 75, 80, 85, 90, 95, 98, or 99% sequence identity to SEQ ID NO:12 and retaining binding affinity for intracellular PHA granules.

In some embodiments, the microorganism may overexpress one or more PHA degradation gene(s), such as, but not limited to, phaY, phaZ, and/or hbd, resulting in reduced production of PHA (e.g., PHB) or PHA with an altered molecular weight distribution or increased digestibility. For example, overexpression may include alteration of one or more regulatory sequence(s) (e.g., increase in promoter strength to increase transcription), improvement in ribosome binding sequence to increase translation, or increase in gene copy number. Alternatively, or additionally, the microorganism may be transformed with exogenous phaY, phaZ, and/or hbd sequences, either added to a microorganism that does not express these genes or as additional copies or higher activity enzymes to a microorganism that does possess endogenous copies of these genes.

In some embodiments, the microorganism overexpresses the phaY polynucleotide sequence depicted in SEQ ID NO:17 or SEQ ID NO:19 or SEQ ID NO:25 or SEQ ID NO:31 or SEQ ID NO:40 or SEQ ID NO:41 (e.g., by alteration of one or more regulatory sequence(s), resulting in increased expression) or a polynucleotide having at least about 30, 35, 40, 45, 50, 55, 60, 65, 70, 75, 80, 85, 90, 95, 98, or 99% sequence identity to SEQ ID NO:17 or SEQ ID NO:19 or SEQ ID NO:25 or SEQ ID NO:31 or SEQ ID NO:40 or SEQ ID NO:41. In some embodiments, the microorganism overexpresses a polynucleotide that encodes PHA oligomer hydrolase, e.g., 3-hydroxybutyrate oligomer hydrolase amino acid sequence, for example, the amino acid sequence depicted in SEQ ID NO: 18 or SEQ ID NO:20 or SEQ ID NO:26 or SEQ ID NO:32 or that encodes an amino acid sequence having at least about 30, 35, 40, 45, 50, 55, 60, 65, 70, 75, 80, 85, 90, 95, 98, or 99% sequence identity to SEQ ID NO:18 or SEQ ID NO:20 or SEQ ID NO:26 or SEQ ID NO:32 and retaining PHA oligomer hydrolase, e.g., 3-hydroxybutyrate oligomer hydrolase enzyme activity (e.g., endo- or exo-PHA oligomer cleavage activity).

In some embodiments, the microorganism overexpresses the phaZ polynucleotide sequence depicted in SEQ ID NO:13 or SEQ ID NO:15 or SEQ ID NO:23 or SEQ ID NO:27 or SEQ ID NO:29 or SEQ ID NO:36 or SEQ ID NO:38 or SEQ ID NO:39 or SEQ ID NO:44 (e.g., by alteration of one or more regulatory sequence(s), resulting in increased expression) or a polynucleotide having at least about 30, 35, 40, 45, 50, 55, 60, 65, 70, 75, 80, 85, 90, 95, 98, or 99% sequence identity to SEQ ID NO:13 or SEQ ID NO:15 or SEQ ID NO:23 or SEQ ID NO:27 or SEQ ID NO:29 or SEQ ID NO:36 or SEQ ID NO:38 or SEQ ID NO:39 or SEQ ID NO:44. In some embodiments, the microorganism overexpresses a polynucleotide that encodes a PHA depolymerase enzyme, for example, the amino acid sequence depicted in SEQ ID NO:14 or SEQ ID NO:16 or SEQ ID NO:24 or SEQ ID NO:28 or SEQ ID NO:30 or SEQ ID NO:37 or SEQ ID NO:45 or that encodes an amino acid sequence having at least about 30, 35, 40, 45, 50, 55, 60, 65, 70, 75, 80, 85, 90, 95, 98, or 99% sequence identity to SEQ ID NO:14 or SEQ ID NO:16 or SEQ ID NO:24 or SEQ ID NO:28 or SEQ ID NO:30 or SEQ ID NO:37 or SEQ ID NO:45 and retaining PHA depolymerase activity, e.g., endo- or exo-PHA oligomer cleavage activity, e.g., PHA degradation via thiolysis.

In some embodiments, the microorganism overexpresses the hbd polynucleotide sequence depicted in SEQ ID NO:21 (e.g., by alteration of one or more regulatory sequence(s), resulting in increased expression) or a polynucleotide having at least about 30, 35, 40, 45, 50, 55, 60, 65, 70, 75, 80, 85, 90, 95, 98, or 99% sequence identity to SEQ ID NO:21. In some embodiments, the microorganism overexpresses a polynucleotide that encodes 1-hydroxybutyrate dehydrogenase amino acid sequence, for example, the amino acid sequence depicted in SEQ ID NO:22 or that encodes an amino acid sequence having at least about 30, 35, 40, 45, 50, 55, 60, 65, 70, 75, 80, 85, 90, 95, 98, or 99% sequence identity to SEQ ID NO:22 and retaining 1-hydroxybutyrate dehydrogenase enzyme activity.

In some embodiments, the microorganism overexpresses the phaM polynucleotide sequence depicted in SEQ ID NO:42 (e.g., by alteration of one or more regulatory sequence(s), resulting in increased expression) or a polynucleotide having at least about 30, 35, 40, 45, 50, 55, 60, 65, 70, 75, 80, 85, 90, 95, 98, or 99% sequence identity to SEQ ID NO:42. In some embodiments, the microorganism overexpresses a polynucleotide that encodes PHA granule associated amino acid sequence, for example, the amino acid sequence depicted in SEQ ID NO:43 or that encodes an amino acid sequence having at least about 30, 35, 40, 45, 50, 55, 60, 65, 70, 75, 80, 85, 90, 95, 98, or 99% sequence identity to SEQ ID NO:43 and retaining the ability to associate with PHA granules.

### Naturally Occurring Microorganisms

In some embodiments, a naturally occurring microorganism that produces PHA (e.g., PHB) is used in methods for producing biomass described herein. The naturally occurring microorganisms are cultured, for example, in a bioreactor with defined culture growth medium and carbon source(s). Culture conditions are chosen to alter the PHA production level and/or protein level and/or PHA:protein ratio from the levels of these substances that are produced under naturally occurring conditions.

In some embodiments, the microorganism is a naturally occurring species of the genus Methylomonas, Methylobacter, Methylococcus, Methylosinus, Methylocyctis, Methylomicrobium, Methanomonas, Methylophilus, Methylobacillus, Methylobacterium, Hyphomicrobium, Xanthobacter, Bacillus, Paracoccus, Nocardia, Arthrobacter, Rhodopseudomonas, Pseudomonas, Candida, Hansenula, Pichia, Torulopsis, Vibrio, Escherichia, Alcaligenes, Ralstonia, Rhodobacter, Saccharomyces, Cupriavidus, Sinorhizobium, Mucor, Bradyrhizobium, Yarrowia, Azotobacter, Synechocystis, Rhodotorula, Aeromonas, Magnetospirillum, Haloferax, Caryophanon, or Allochromatium.

In some embodiments, the naturally occurring microorganism is Methylobacterium extorquens (e.g., strains AM1, DM4, CM4, PA1, DSMZ 1340), Methylobacterium populi (BJ001), Methylobacterium radiotolerans, Methylobacterium nodulans, Methylobacterium sp 4-46, or other Methylobacterium species.

In some embodiments, the naturally occurring microorganism is a methylotrophic bacterium.

### Transformation of Microorganisms

Numerous transformation protocols and constructs for introducing and expressing exogenous polynucleotides in host cells are known in the art.

In certain embodiments, genetic modifications will take advantage of freely replicating plasmid vectors for cloning. These may include small IncP vectors developed for use in Methylobacterium. These vectors may include pCM62, pCM66, or pHC41 for cloning. (Marx, C. J. and M. E. Lidstrom Microbiology (2001) 147: 2065-2075; Chou, H.-H. et al. PLoS Genetics (2009) 5: e1000652)

In certain embodiments, genetic modifications will take advantage of freely replicating expression plasmids such as pCM80, pCM160, pHC90, or pHC91. (Marx, C. J. and M. E. Lidstrom Microbiology (2001) 147: 2065-2075; Chou, H.-H. et al. PLoS Genetics (2009) 5: e1000652)

In certain embodiments, genetic modifications will utilize freely replicating expression plasmids that have the ability to respond to levels of inducing molecules such as cumate or anhydrotetracycline. These include pHC115, pLC290, pLC291. (Chou, H.-H. et al. PLoS Genetics (2009) 5: e1000652; Chubiz, L. M. et al. BMC Research Notes (2013) 6: 183)

In certain embodiments, genetic modifications will utilize recyclable antibiotic marker systems such as the cre-lox system. This may include use of the pCM157, pCM158, pCM184, pCM351 series of plasmids developed for use in M. extorquens. (Marx, C. J. and M. E. Lidstrom BioTechniques (2002) 33: 1062-1067)

In certain embodiments, genetic modifications will utilize transposon mutagenesis. This may include mini-Tn5 delivery systems such as pCM639 (D'Argenio, D. A. et al. J Bacteriol (2001) 183: 1466-1471) demonstrated in M. extorquens. (Marx, C. J. et al. J Bacteriol (2003) 185: 669-673)

In certain embodiments, genetic modifications will utilize expression systems introduced directly into a chromosomal locus. This may include pCM168, pCM172, and pHC01 plasmids developed for M. extorquens AM1. (Marx, C. J. and M. E. Lidstrom Microbiology (2001) 147: 2065-2075; Lee, M.-C. et al Evolution (2009) 63: 2813-2830)

In certain embodiments, genetic modifications will utilize a sacB-based system for unmarked exchange of alleles due to the sucrose sensitivity provided by sacB expression. This may include the pCM433 vector originally tested with M. extorquens. (Marx, C. J. et al. BMC Research Notes (2008) 1:1)

### Microbial Cultures

Methods for producing biomass are provided. The methods include culturing a microorganism as described herein in a culture medium under conditions suitable for growth of the microorganism and production of biomass that contains PHA:protein in a weight ratio of about 1:1000 to about 2:1. In some embodiments, the PHA is PHB.

The microorganism may be naturally occurring, and the culture conditions are chosen to affect the level of PHA produced in the culture and/or the ratio of PHA:protein produced in the culture, or the microorganism may be non-naturally occurring and engineered or selected for modified, i.e., reduced, PHA production and/or altered ratio of PHA:protein produced, as described herein. In some embodiments, the microorganism may be non-naturally occurring, as described herein, and the culture conditions may be selected to further alter the level of PHA, the ratio of PHA:protein produced, the PHA digestibility, and/or the molecular weight distribution of the PHA polymers.

In embodiments in which the microorganism also produces one or more carotenoid compound(s) (e.g., a microorganism that has been genetically modified or artificially pre-selected to produce elevated levels of one or more carotenoid compound(s)), biomass that includes PHA and the one or more carotenoid compound(s) is produced.

In various embodiments, the culture conditions may include one or more of: aeration of the culture medium (e.g., resulting in a dissolved oxygen concentration of about 5% to about 50%); temperature of the culture medium (e.g., temperature of about 20° C. to about 50° C.); carbon source comprising, consisting of, or consisting essentially of one or more alcohol(s) (e.g., methanol, ethanol, glycerol, or a combination thereof); and semi-continuous or continuous fermentation conditions.

In some embodiments, the culture conditions that result in a desired PHA level and/or PHA:protein ratio include aeration of the culture medium. For example, the culture medium may be aerated to provided dissolved oxygen at about 5% to about 50%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 5% to about 25%, about 10% to about 35%, about 20% to about 40%, or about 25% to about 50%, or any of at least about 5%, 10%, 15%, 20%, 25%, 30%, 35%, 40%, 45%, or 50%.

In some embodiments, the culture conditions that result in a desired PHA level and/or PHA:protein ratio include temperature of the culture medium. For example, the culture medium may be maintained at a temperature of about 20° C. to about 50° C., about 20° C. to about 25° C., about 25° C. to about 30° C., about 30° C. to about 35° C., about 35° C. to about 40° C., about 40° C. to about 45° C., about 45° C. to about 50° C., about 20° C. to about 30° C., about 30° C. to about 40° C., about 40° C. to about 50° C., about 20° C. to about 35° C., about 25° C. to about 40° C., about 30° C. to about 45° C., about 35° C. to about 50° C., about 20° C. to about 40° C., about 30° C. to about 50° C., or any of about 20, 25, 30, 35, 40, 45, or 50° C.

The culture medium includes carbon source(s), nitrogen source(s), inorganic substances (e.g., inorganic salts), and any other substances required for the growth of the microorganism (e.g., vitamins, amino acids, etc.).

The carbon source may include sugars, such as glucose, sucrose, lactose, fructose, trehalose, mannose, mannitol, and maltose; organic acids, such as acetic acid, lactic acid, fumaric acid, citric acid, propionic acid, malic acid, pyruvic acid, malonic acid, succinic acid and ascorbic acid; alcohols, such as methanol, ethanol, propanol, butanol, pentanol, hexanol, isobutanol, and glycerol; oil or fat, such as soybean oil, rice bran oil, olive oil, corn oil, sesame oil, linseed oil, and the like. The amount of the carbon source added varies according to the kind of the carbon source, for example, about 1 to about 100 g, or about 2 to about 50 g per liter of medium.

In some embodiments, a C1 carbon substrate is provided to a microorganism that is capable of converting such a substrate to organic products (e.g., microorganisms of the genera Methylobacterium, Methylomonas, Methylobacter, Methylococcus, Methylosinus, Methylocyctis, Methylomicrobium). In certain embodiments, the C1 carbon substrate is selected from methane, methanol, formaldehyde, formic acid, methylated amines, methylated thiols, and carbon dioxide. In certain embodiments, the C1 carbon substrate is selected from methanol, formaldehyde, and methylated amines. In certain embodiments, the C1 carbon substrate is methanol.

In some embodiments, the culture conditions that result in a desired PHA level and/or PHA:protein ratio include a carbon source that comprises, consists of, or consists essentially of one or more alcohol(s), such as, but not limited to, methanol, ethanol, and/or glycerol.

The nitrogen source may include potassium nitrate, ammonium nitrate, ammonium chloride, ammonium sulfate, ammonium phosphate, ammonia, urea, and the like, alone or in combination. Amount of the nitrogen source added varies according to the kind of the nitrogen source, for example, about 0.1 g to about 30 g, or about 1 g to about 10 g per liter of medium.

Inorganic salts may include potassium dihydrogen phosphate, dipotassium hydrogen phosphate, disodium hydrogen phosphate, sodium dihydrogen phosphate, magnesium sulfate, magnesium chloride, ferric sulfate, ferrous sulfate, ferric chloride, ferrous chloride, manganese sulfate, manganese chloride, zinc sulfate, zinc chloride, cupric sulfate, calcium chloride, calcium carbonate, sodium carbonate, sodium sulfate, and the like, alone or in combination. Amount of inorganic salt varies according to the kind of the inorganic salt, for example, about 0.00001 to about 10 g per liter of medium.

Special required substances, for example, vitamins, nucleic acids, yeast extract, peptone, meat extract, malt extract, corn steep liquor, soybean meal, dried yeast etc., may be included alone or in combination. Amount of the special required substance used varies according to the kind of the substance, for example, about 0.2 g to about 200 g, or about 3 to about 10 g per liter of medium.

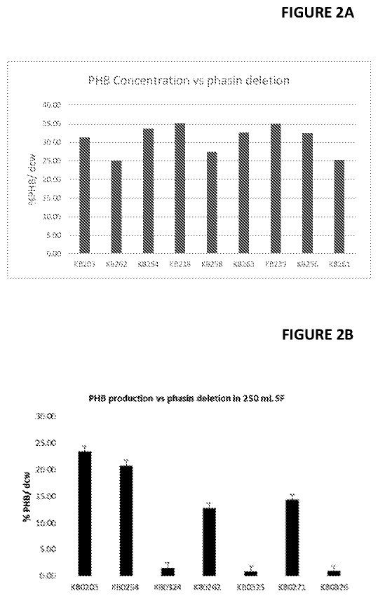

In some embodiments, the pH of the culture medium is adjusted to pH about 2 to about 12, or about 6 to about 9. The medium may further include one or more buffer(s) to maintain the culture at the desired pH. Numerous buffers are known in the art and include phosphate, carbonate, acetate, PIPES, HEPES, and Tris buffers. A suitable buffer for a given microorganism can easily be determined by one of ordinary skill in the art. For Methylobacterium, a common medium, described by Lee, et al. (2009) Evolution 63:2813-2830, is a phosphate buffered medium that consists of 1 mL of trace metal solution (to 1 liter of deionized water the following are added in this order: 12.738 g of EDTA disodium salt dihydrate, 4.4 g of ZnS0-7H₂0, 1.466 g of CaCI₂-2H₂O, 1.012 g of MnCI₂-4H₂0, 0.22 g of (NH₄)₆Mo₇O₂₄-4H₂0, 0.314 g of CuSO₄-5H₂O, 0.322 g of CoCl₂-6H₂0, and 0.998 g of Fe₃(SO₄)₂-7H₂0; pH 5.0 is maintained after every addition), 100 mL of phosphate buffer (25.3 g of K₂HPO₄ and 22.5 g of NaH₂PO₄ in 1 liter of deionized water), 100 mL of sulfate solution (5 g of (NH₄)₂(SO₄) and 0.98 g of Mg(SO₄)₂ in 1 liter of deionized water), and 799 mL of deionized water. All components are heat sterilized separately and then pooled together. An alternative medium recently developed for use with Methylobacterium extorquens takes advantage of an organic buffer and has a citrate-chelated trace metal mix. Culturing is carried out at temperature of 15° to 40° C., and preferably 20° to 35° C., usually for 1 to 20 days, and preferably 1 to 4 days, under aerobic conditions provided by shaking or aeration/agitation. Common practice with Methylobacterium is at 30° C. The protocol for making M-PIPES medium is described in Table S1 of Delaney et al. (2013) PLoS One 8:e62957. FIG. 2 in U.S. Ser. No. 61/863,701 shows an exemplary recipe for medium optimized for use with M. extorquens.

In order to generate dense cultures of microorganisms, such as Methylobacterium, it may be advantageous to use a fed-batch method. Methanol can be tolerated well at 0.5-1% v/v (˜120-240 mM), and thus this step size of addition can be used repeatedly. Critically, pH levels drop during culturing on methanol, such that the use of a base such as KOH or NaOH would be important to maintain the pH around 6.5. Aeration can be achieved via physical agitation, such as an impeller, via bubbling of filtered air or pure oxygen, or in combination. In order to reduce production costs, the buffer can be replaced from phosphates or PIPES to a carbonate-buffered medium.

In some embodiments, a “fill and draw” method is used, in which a portion of the culture medium (e.g., about 10% to about 90%) is removed when the culture reaches a desired optical density at 600 nm (e.g., about 50 to about 200), followed by replacement with an equivalent amount of fresh medium, thereby maintaining PHA (e.g., PHB) at a relatively constant level in the culture, and thereby resulting in biomass that contains a desired level of PHA and/or a desired PHA:protein ratio.

In some embodiments, a “continuous” method is used, in which fresh medium is continuously added, while culture medium and microorganisms are continuously removed at the same rate, keeping the culture volume relatively constant, thereby resulting in biomass that contains a desired level of PHA, PHA molecular weight distribution, digestibility, and/or a desired PHA:protein ratio.

Microbial cells may be separated from the culture, for example, by a conventional means such as centrifugation or filtration. The cells may be isolated whole, or may be lysed to release their contents for extraction or further processing. The cells or the medium may be subjected to an extraction with a suitable solvent.

### Compositions

Compositions are provided for use as feed in aquaculture, or as animal feed, or as human nutritional supplements containing processed or unprocessed biomass from microorganism cells cultured as described herein, as are methods of preparation of the feed or nutritional supplement compositions.

The feed compositions or nutritional supplements include PHA (e.g., PHB) containing biomass, produced by culturing one or more microorganism(s) as described herein, i.e., produced by culturing a non-naturally occurring microorganism as described herein and/or by applying culture conditions to a non-naturally occurring or naturally occurring microorganism that result in a desired PHA level, PHA molecular weight distribution, digestibility, and/or PHA:protein ratio, as described herein.

In certain embodiments, biomass that is incorporated into a feed or nutritional supplement composition can be in a dry, or substantially dry, form, e.g., containing less than about 20%, 10%, 5%, or 2% of moisture. In certain embodiments, the cultures are isolated by removing substantially all supernatant, such as by filtering, sedimentation, or centrifugation. In certain embodiments, the collection of cultures and further processing of biomass excludes a bacterial lysis step, e.g., by use of detergents or ultrasound. In certain embodiments, the processed microbial cells maintain substantially whole cell membranes. In some embodiments, a substantial portion (e.g., more than about 5%, 10%, 20%, 30%, 50%, or 80%) of bacterial cells may maintain viability in the processed biomass.

The feed composition may contain at least about 1% of the biomass by weight. In certain embodiments, the feed composition is optimized for consumption by fish, seafood, humans, poultry, swine, cattle or other animals. For example, the feed may include one or more of EPA, DHA, and one or more essential amino acids.

Methods for preparing a feed composition are also provided. In some embodiments, the method includes: (a) culturing in an appropriate medium at least one non-naturally occurring microorganism as described above; (b) concentrating the medium to provide a biomass; (c) optionally providing additional feed components; and (d) producing the feed composition from the biomass. In certain embodiments, step (b) includes centrifugation. In certain embodiments, step (b) includes allowing the biomass to settle. In certain embodiments, step (b) includes filtration. In certain embodiments, the method further includes a pre-treatment of the biomass after step (a) with a chemical agent (e.g., a surfactant or solvent) to disrupt the cell membranes of the biomass. In certain embodiments, the method further includes mechanical disruption of the cell membranes of the biomass after step (a).

Examples of feedstuffs into which single cell protein enriched with PHA (e.g., PHB), produced as described herein, may be incorporated include, for example, pet foods, such as cat foods, dog foods and the like, feeds for aquarium fish, cultured fish or crustaceans, etc., feed for farm-raised animals (including livestock and further including fish or crustaceans raised in aquaculture). The state of the biomass can be in whole cell, lysed or partially processed. PHA-enriched biomass or PHA-enriched protein, produced as described herein can also be incorporated into food or vitamin supplements for human consumption, optionally with additional caloric or nutritional supplements. Food or feed material that includes PHA or biomass that includes PHA, produced as described herein, is incorporated is preferably palatable to the organism that is the intended recipient. This food or feed material may have any physical properties currently known for a food material (e.g., solid, liquid, soft). In some embodiments, feed produced as described herein will undergo a pelletization process, e.g., through a hot or cold extrusion process at an inclusion rate of less than about 1%, 5%, 10%, 20%, 25%, 30%, 40%, 50%, 60%, or 75%. In other scenarios, PHA-enriched biomass or PHA-enriched protein, produced as described herein, can be consumed directly at 100% or combined with another substance in the form of liquid, baked goods or other to form, including but not limited to, various types of tablets, capsules, drinkable agents, gargles, etc.

In some embodiments, the feed or nutritional composition or the biomass includes additional native or heterologous PHA degrading enzymes.

In some embodiments, the feed or nutritional composition or the biomass that is incorporated into the feed or nutritional composition includes any of about 0.1% to about 0.5%, about 0.5% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, or about 45% to about 50% PHA (e.g., PHB) by weight, and any of about 35% to about 40%, about 40% to about 45%, about 45% to about 50%, about 50% to about 55%, about 55% to about 60%, about 60% to about 65%, about 65% to about 70%, or greater than about 70% protein by weight.

In some embodiments, the feed or nutritional composition or the biomass that is incorporated into the feed or nutritional composition includes PHA (e.g., PHB) and protein at a PHA:protein ratio that is about 1:1000 to about 2:1, about 1:1000 to about 1:6, or about 1:1 to about 2:1. In some embodiments, the PHA:protein ratio in the feed composition or biomass is about 1:1000 to about 1:500, about 1:500 to about 1:100, about 1:100 to about 1:50, about 1:50 to about 1:10, about 1:10 to about 1:6, about 1:6 to about 1:2, or about 1:2 to about 1:1, or about 1:1 to about 2:1.

In some embodiments, the feed or nutritional composition or the biomass has PHA with increased bioavailability. In some embodiments the PHA polymers have reduced or altered average molecular weight (Mw, Mn, Mp, or Mz), increased polydispersity, or increased digestibility, e.g., in comparison to a wild type or parent strain and/or a strain grown under different culture conditions than those taught herein, e.g., culture conditions different than those described herein to alter the level of PHA, the ratio of PHA:protein produced, the PHA digestibility, and/or the molecular weight distribution of the PHA polymers.

In some embodiments, a feed or nutritional composition as described herein includes a plurality of microorganisms that each produce PHA (e.g., PHB) at a different level (e.g., one or more non-naturally occurring microorganism(s) that have include mutation(s) for reduced or enhanced PHA production, and/or one or more naturally occurring microorganism(s) that have been cultured under conditions for reduced or enhanced PHA production, as described herein), and the combination of microorganism biomass in the composition results in desired PHA and protein concentrations. For example, the plurality of microorganisms may be incorporated into a feed or nutritional composition to produce a composition that includes any of about 0.1% to about 0.5%, about 0.5% to about 1%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, or about 45% to about 50% PHA (e.g., PHB) by weight, and any of about 35% to about 40%, about 40% to about 45%, about 45% to about 50%, about 50% to about 55%, about 55% to about 60%, about 60% to about 65%, about 65% to about 70%, or greater than about 70% protein by weight. For example, the plurality of microorganisms may be incorporated into a feed or nutritional composition to produce a composition that includes PHA (e.g., PHB) and protein at a PHA:protein ratio that is about 1:1000 to about 2:1, about 1:1000 to about 1:6, or about 1:1 to about 2:1. In some embodiments, the PHA:protein ratio in the feed composition or biomass is about 1:1000 to about 1:500, about 1:500 to about 1:100, about 1:100 to about 1:50, about 1:50 to about 1:10, about 1:10 to about 1:6, about 1:6 to about 1:2, or about 1:2 to about 1:1, or about 1:1 to about 2:1.

In some embodiments, a feed or nutritional composition as described herein includes a plurality of microorganisms that produce PHA and/or additional native or heterologous PHA degrading enzymes.

Methods of producing fish or seafood are also provided, including farming fish or seafood, and providing a diet, which includes a feed composition as described herein, to the fish or seafood.

### Enhanced Survivability

Methods are provided for improving survivability of a livestock or aquaculture (e.g., seafood or fish) animal. The methods include feeding the animal a feed composition as described herein, e.g., a feed composition that includes PHA:protein or biomass that includes PHA:protein in a weight ratio of about 1:1000 to about 2:1, wherein survivability is increased by at least about 1%, 5%, 10%, 15%, 20%, 25%, 30%, 35%, 40%, 45%, 50%, 55%, 60%, 75%, 70%, 75%, 80%, 85%, 90%, 100%, 110%, 120%, 130%, 140%, 150%, 160%, 170%, 180%, 190%, or 200% or more in comparison with a feed composition that does not include PHA. In some embodiments, the PHA is PHB.

The following examples are intended to illustrate, but not limit, the invention.

### EXAMPLES

### Example 1. Deletion of Phasins

Sequence analysis of M. extroquens PA1 genome was used to identify three putative phasins: Mext_0493, Mext_2223, and Mext_2560. According to sequence homology, Mext_2223 matches AM1 gap11 and Mext_2560 matches gap20. Deletion of Mext_2223 resulted in a dramatic decrease in PHB production (See FIG. 2B): about 1% PHB produced in M. extorquens KB0324 (about 95% decrease), while deletion of Mext_2560 resulted in a 20-50% decrease, depending on the culture conditions, such as volume, aeration (less DO leads to an increase in PHB), temperature (temperature over 30° C. increases PHB) and feeding strategy (nutrient limitation leads to an increase).

Methylobacterium extorquens Strain Genotypes:

KB0203 (PA1 derivative)

### KB0254: KB0203 ΔMext_0493

### KB0262: KB0203 ΔMext_2560

### KB0271: KB0203 ΔMext_0493 ΔMext_2560

### KB0324: KB0203 ΔMext_0493 ΔMext_2223

### KB0325: KB0203 ΔMext_2560 ΔMext_2223

### KB0326: KB0203 ΔMext_0493 ΔMext_2560 ΔMext_2223

KB0218: derivative of KB0203

### KB0258: KB0218 ΔMext_2560

### KB0253: KB0218 ΔMext_0493

KB0239: derivative of KB0203

### KB0256: KB0239 ΔMext_0493

### KB0261: KB0239 ΔMext_2560

Results are shown in FIGS. 2A and 2B. The data is from 250 mL shake flask experiments, with growth media SP5 (salt media) supplemented with 0.2% methanol v/v. Data were confirmed at 1 L scale.

### Example 2. Effect of Culture Conditions on PHB and Protein Production

### Aeration

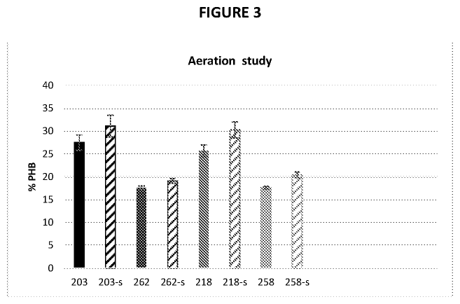

FIG. 3 shows the results of an experiment investigating the effect of decrease in oxygen (aeration). An aeration study was conducted in shake flask at 32° C. with SP5 (salt and minerals) media. Strains KB0203, KB0262, KB0218 and KB0258 were cultivated in either 25 ml SP5+0.4% Methanol in 125 ml flask or 15 ml SP5+0.4% Methanol in 100 ml small mouth flask which resulted in a decrease in oxygenation. “s” indicates use of a small mouth flask in the graph. At the end of fermentation, cell sample was centrifuged at 4° C., 4000 rpm during 20 minutes. Pellets were then washed once in 0.05× Phosphate Buffer Saline (PBS) solution, centrifuged and lyophilized Intracellular PHB was converted to crotonic acid by treating approximately 5 mg of lyophilized cells with 0.5 mL concentrated sulfuric acid, and holding at 100° C. for 30 minutes. The solution was then cooled, diluted with 2.5 mL MilliQ water, and centrifuged at 4300 rpm for 20 minutes. The supernatant was then diluted in preparation for UPLC analysis. Diluted samples were analyzed on a Waters 3100 Mass Detector UPLC-MS at 0.5 mL/min on a 50 mm×1.7 C18 UPLC column using 60% MilliQ water+0.1% Formic Acid and 40% Methanol+0.1% Formic Acid. The peak areas of samples were compared to the peaks of PHB standards that were similarly hydrolyzed. PHB is reported as a % of dry cell weight (dcw).

### Temperature

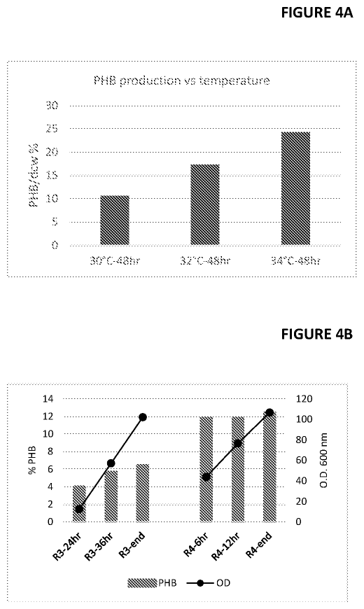

Strain KB0203 was grown for 72 h in a 1 L DASGIP® parallel bioreactor system's vessel containing CHOI4 medium (Bourque, et al. (1995) Appl Microbiol Biotechnol 44:367-376) with an initial concentration of Dow Corning AFE1520 antifoam of 140 ppm. The initial OD600 is set at 0.2, the DO at 15% and methanol concentration is kept constant at 0.2% using Intempco control system. Temperature set points are 30, 32, 34 and 36° C. production. At end of fermentation, cell sample was centrifuged at 4° C., 4000 rpm during 20 minutes. Pellets were then washed once 0.05× Phosphate Buffer Saline (PBS) solution, centrifuged and lyophilized. Dry cells were weighted to obtain ˜5 mg of material. PHB analysis was performed as described above. About 4-5 gm of lyophilized culture is sent to New Jersey Feed Lab (NJFL, Inc. Trenton, N.J.) for proximate analysis. As shown in FIG. 4A, production of PHB increases as temperature increases above 30° C. KB0203 did not grow well at 36° C. Strain KB0258, that is producing ˜25-50% less PHB compared to KB0203 was grown for ˜40 h in a 1 L DASGIP® parallel bioreactor system's vessel as described above. Temperature was set at 30 or 32° C. An increased production of PHB was again observed when temperature is above 30° C. (FIG. 4B).

### Fill and Draw

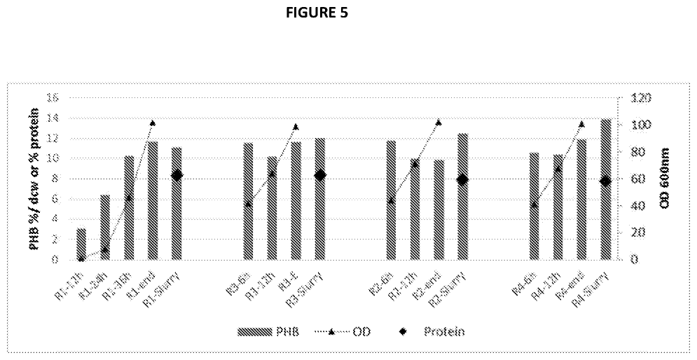

A “fill and draw” experiment was performed to investigate effect on PHB production. Strain KB0203 was cultivated in a 1 L DASGIP® parallel bioreactor system's vessel containing CHOI4 medium as described above. Once the reactor reaches an optical density of 100 at 600 nm, the reactor was stopped and one fifth of the active reactor volume (150 ml) was used to inoculate the next reactor, containing 600 ml of the fresh CHOI4+0.2% methanol. Throughout the experiment, withdrawn volumes were replaced with fresh CHOI4 medium. Samples were centrifuged and cell pellets were washed once with PBS 0.05×. Samples were then lyophilized and analyzed for PHB and protein content as described above. This fill and draw strategy allowed maintenance of the level of PHB at about 12% throughout the fermentation process while generating biomass. The results are shown in FIG. 5.

### Correlation of PHB Level and Protein Content

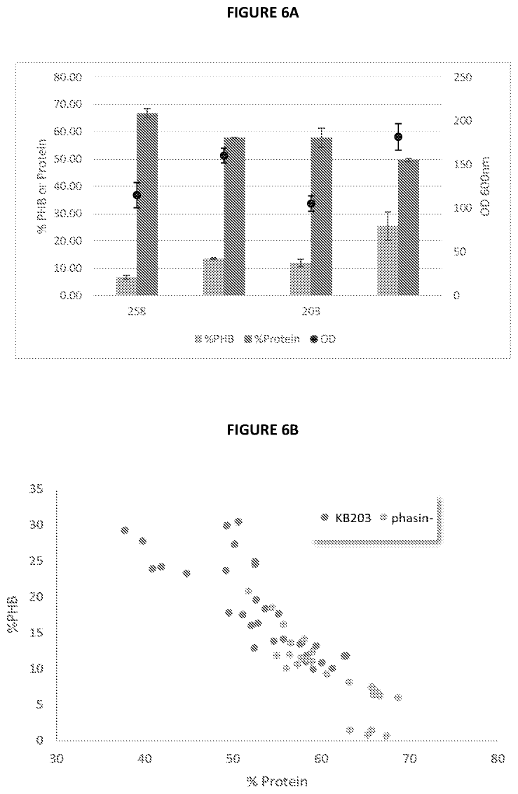

Decreasing PHB production was shown to increase protein content in cells, as shown in FIGS. 6A and 6B. FIG. 6A represent the compiled average of % PHB and % protein obtained with strains KB0203 and KB0258 across various fermentation experiments. It shows a strong correlation between low PHB level and high protein content: when PHB levels dropped below 5-6%, protein levels reached about 70%. PHB levels are controlled either by genetic modification (deletion of phasin in KB0258) or fermentation conditions.

To further explore the correlation between protein content and PHB, we compared multiple results from cells grow in fermenters under different conditions. Wild type KB203 was compared to a group of strains with one of more deletions in the phasin encoding genes. FIG. 6B shows the correlation between percent protein and percent PHB in these strains.

### Impact of Methanol-Ethanol Co-Feed on PHB Production.

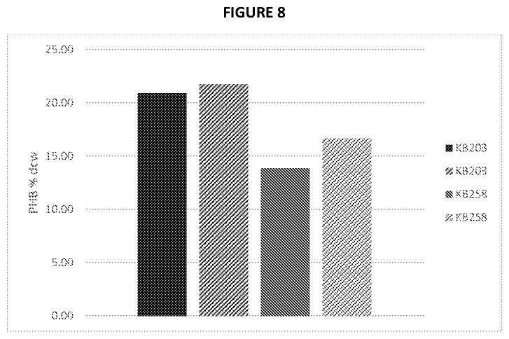

Strains KB0203 and KB0258 were cultivated in 25 ml media (SP5+Tmp) in 250 mL flasks at 30° C. Cultures were fed 0.5% methanol or a mixture of 0.3% methanol+0.1% ethanol at time 0, 16, 24, 40 and 48 h hours. PHB was measured as an endpoint at 68 hours. The results are shown in FIG. 8. Growth on methanol-ethanol co-feed resulted in an increase in PHB production.

### Example 3. Effect of PHB on Survivability

### Shrimp Data

Survival of Pacific white shrimp, Litopenaeus vannamei, on diets that contained PHB in bacterial biomass (BB) versus diets without PHB were investigated. 3 trials of 6 weeks each were conducted. The trial 1 utilized 3 treatments with 4 replicates in each treatment. It was conducted in a semi-closed recirculation system. Juvenile shrimp were stocked into 12 tanks with 8 shrimp in each aquarium (160 L). Based on historical results, a fixed ration was calculated assuming a 1.8 feed conversion ratio and a doubling in size the first two weeks and 0.8-1.3 g week⁻¹ thereafter. The trial 2 utilized 6 treatments with 4 replicates in each treatment. Juvenile shrimp were stocked into 24 tanks with 10 shrimp in each aquarium (80 L). Shrimp were counted to readjust daily feed input on a weekly basis. In trial 2 and trial 3, the recirculating system consisted of 24 aquaria (135 L) connected to a common reservoir, biological filter, bead filter, fluidized biological filter and recirculation pump. Four replicate groups of shrimp (In trial 2: 0.98 g initial mean weight, 10 shrimp/tank; In trial 3: 0.15 g initial mean weight, 10 shrimp/tank) were offered diets using standard feeding protocol over 6 weeks.

At the conclusion of each growth trial, shrimp were counted and group-weighted. Mean final weight, Feed Conversion Ratio (FCR) (feed offered/(final weight−initial weight)), Weight Gain (WG) ((final weight−initial weight)/initial weight×100%), biomass, and survival were determined.

In trials 1 and 2, test diets were formulated to be isonitrogenous and isolipidic (35% protein and 8% lipid). In trial 1, three experimental diets (T₁D₁-T₁D₃) were formulated to contain increasing levels (0, 6, and 12%) of BB in replacement of Soy Bean Meal (SBM).

In trial 2, in order to confirm the results in trial 1 and investigate the effects of low inclusion levels of BB, six experimental diets (T₂D₁-T₂D₆) were formulated to supplement with increasing levels (0, 1, 2, 4, 6, and 12%) of BB as a replacement of Soy Bean Meal (SBM)

In trial 3, five experimental diets (T₃D₁-T₃D₅) were formulated. T₃D₁, T₃D₂, and T₃D₄ were the same as diets in trial 2 that utilized 0, 60, and 120 g kg⁻¹ BB to replace soybean meal (SBM). T₃D₃ and T₃D₅ included BB to replace the same ratio of SBM as T₃D₂ and T₃D₄, respectively, on a digestible protein basis.

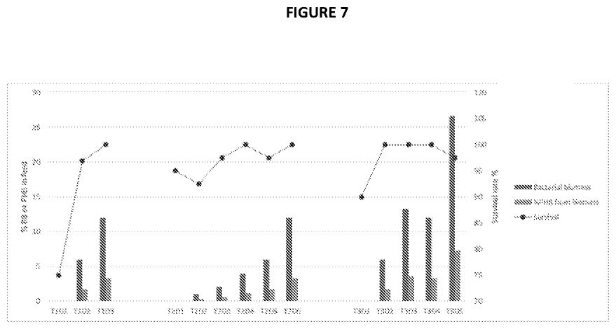

The results are shown in Table 1 and FIG. 7.

TABLE 1

| | T1D1 | T1D2 | T1D3 | T2D1 | T2D2 | T2D3 | T2D4 | T2D5 | T2D6 | T3D1 | T3D2 | T3D3 | T3D4 | T3D5 |
|---|---|---|---|---|---|---|---|---|---|---|---|---|---|---|
| BB % | 0 | 6 | 12 | 0 | 1 | 2 | 4 | 6 | 12 | 0 | 6 | 13.3 | 12 | 26.6 |
| Final | 49.25 | 53.85 | 45.7 | 79.34 | 84.93 | 84.02 | 85.29 | 75.27 | 58.09 | 42.68 | 43.15 | 45.38 | 38.48 | 35.05 |
| biomass | | | | | | | | | | | | | | |
| Final mean | 8.26 | 6.96 | 5.72 | 8.35 | 9.2 | 8.62 | 8.53 | 7.72 | 5.81 | 4.74 | 4.3 | 4.54 | 3.84 | 3.6 |
| weight (gm) | | | | | | | | | | | | | | |
| Weight | 440.04 | 370.55 | 280.94 | 766.59 | 836.79 | 811.12 | 765.32 | 697.48 | 493.7 | 3160.39 | 2813.38 | 2732.16 | 2438.14 | 2304.94 |
| gain (gm) | | | | | | | | | | | | | | |
| FCR | 1.65 | 1.99 | 2.61 | 1.64 | 1.5 | 1.56 | 1.63 | 1.83 | 2.5 | 1.72 | 1.9 | 1.73 | 2.11 | 2.26 |
| Survival % | 75 | 96.9 | 100 | 95 | 92.5 | 97.5 | 100 | 97.5 | 100 | 90 | 100 | 100 | 100 | 97.5 |

### Example 4

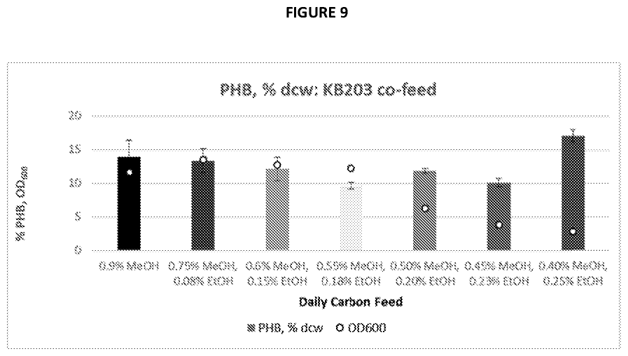

To determine the effects of increasing ethanol on PHB percent, 500 mL cultures of KB0203 were grown in shake flasks with varying amounts of methanol and ethanol. After 65 hours of growth, the culture was harvested and the percent PHB was analyzed as above. Overall, it was observed that additional ethanol leads to decreased levels of PHB up to the point where growth is affected. The results are shown in FIG. 9.

### Example 5

As a water-insoluble polymer of an organic acid, PHB is an ideal nutrient for aquaculture. However, the long polymers of PHB that many bacteria produce may not be fully broken down into digestible organic acids before exiting the digestive track. To increase the digestibility of our bacterial PHB, endogenous and heterologous genes were cloned into pLC291 and driven by the promoters HP1 and pMxaF (SEQ ID NOs: 34 and 35). Several of these genes are from organisms that are capable of breaking down and utilizing PHB as a sole carbon source (Sugiyama, et al. (2004) Cur Microbiol 48:424-7; Hadrick, et al. (2001) J Biol Chem 276:36215-24; Anderson, et al. (1990) Microbiol Rev 54:450-472; Focarete, et al. (1999) Macromolecules 32:4184-4818; Jendrossek, et al. (2002) Annu Rev Microbiol 56:403-32).

These plasmids were introduced into strain KB203; 500 mL cultures were grown in 4 L shake flasks, harvested, and PHB content was determined as described above. Increasing or introducing PhaY, PhaZ, HBD, and phasin proteins led to altered PHB content (See Table 2). Generally, increasing the amount of PHB degradation enzymes led to decreased amounts of PHB. Deletion of phasins or PHB biosynthesis enzymes resulted in decreased amounts of PHB.

Shorter PHB polymers are of interest as they should be degraded more readily by chemical and enzymatic processing, leading to increased availability of 3-hydroxybutyrate. To determine the effects of expression of PHA degrading enzyme genes and the effects of deletion of native phasin proteins, Gel Permeation Chromatography (GPC) was utilized.

To extract the PHB for GPC, 500 mL cultures were grown in 4 L culture flasks and harvested. Neutral lipids and some proteins were removed from the lyophilized cell material by sonicating the biomass material at 30° C. in an equal mixture of methanol and water and subsequently sonicating in pure methanol. Following drying, the PHB was extracted by adding chloroform and sonicating at 60° C. The chloroform extracted PHB was precipitated by adding to cold methanol, pelleted, washed with additional methanol, and dried.

The extracted PHB was dissolved in chloroform to 1 to 10 mg/mL and analyzed by GPC on a Waters Alliance 2695 HPLC system with Photodiode Array Detector (PDA) and refractive index detector (RFID). Waters Styragel HR columns 1,3, and 4 were used for molecular weight determination using 100% tetrahydrofuran (THF) as a mobile phase. Polystyrene standards from a molecular weight of 500 to 400K are used to create the calibration curve. 100 to 250 ul of the extract was injected into the 0.9 ml/min THF stream. The resulting RFID peaks were compared to the polystyrene standards and polymer size (Mn, Mw, Mp) and polydispersity were determined using Waters Empower 2.

Table 3 shows the effects of phasin mutations and expression of endogenous heterologous genes on PHB polymer length and distribution. Deletion of phasin genes led to much lower average PHB polymer length in bacteria grown in either shake flask or fermenters. Expression of PhaY and PhaZ proteins led to decreased average PHB polymer length and increased polydispersity due to an increase in smaller PHB oligomers.

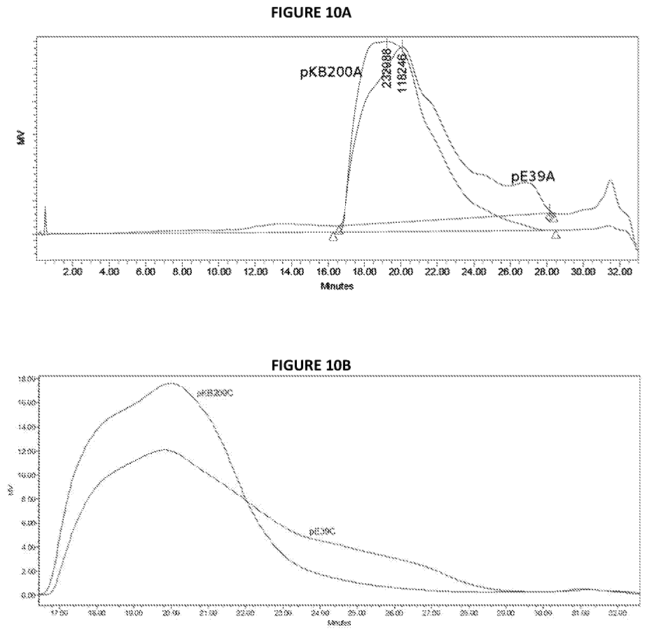

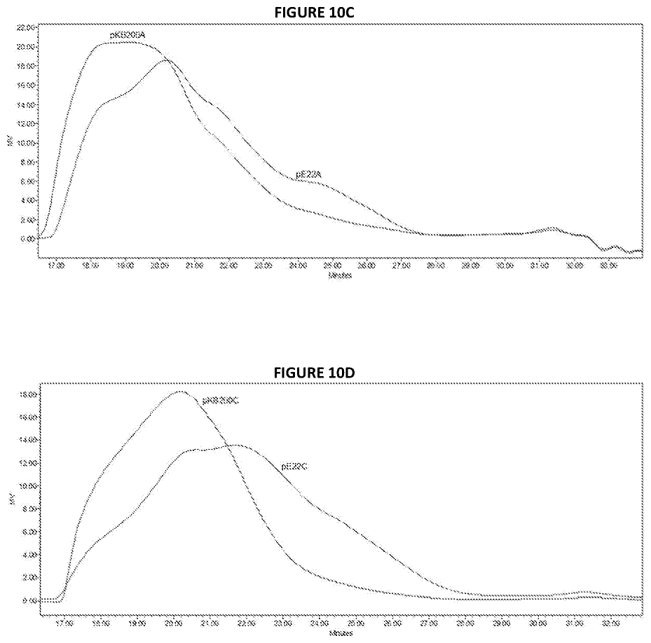

FIGS. 10A-10D show the GPC-RFID trace of PHB extracted from strains expressing PhaZ_Rp (pE22A/C), PhaZ7_Pl (pE39A/C), or a control plasmid (pKB200A/C). Both enzymes increased the amounts of smaller oligomers as seen by the shift in the main peak and the broad shoulder from minutes 21-28 relative to the control strain. Increased expression of PhaZ_Rp or PhaZ7_Pl driven by the stronger pMxaF promoter (SEQ ID NO: 35) led to a larger portion of smaller oligomers (Compare pE # A versus pE # C in FIGS. 10A-D).

To ascertain if the PHB in the strains expressing PhaZ_Rp or PhaZ7_Pl would lead to more digestible polymers, we modified our protocol for determining PHB content described above by reducing the sulfuric acids from 100% to 60%. Using biomass from strains expressing PhaZ_Rp and PhaZ7_Pl, we found that expressing of these enzymes led to higher amounts of crotonic acid than control plasmids in 60% sulfuric acid relative to 100% sulfuric acid (See Table 4). Similar results were seen when comparing KB203 and the carotenoid producing strain KB387, which makes smaller PHB polymers on average (See Table 3). This data demonstrates smaller oligomers of PHA or PHB are more readily broken down to active soluble organic acids.

### Example 6

Different carbon sources including combined feeding of methanol and ethanol can alter PHB content (see above). To find the effects of different carbon sources on PHB, methanol, ethanol, glycerol, formate, acetate, succinate, malate, and combinations thereof were fed to KB203 and an evolved strain KB287 in 5 mL cultures in 20×150 mm tubes. The strains were fed Methanol (M), Ethanol (E), Glycerol (G) at 49.4, 25.7, or 27.4 mM or Formate (F), Acetate (A), Succinate (S), or Malate (Ma) at 10 mM three times before harvesting and analyzing the PHB content as described above. Addition or sole feeding on ethanol, glycerol, formate, succinate, and malate resulted in reduced PHB content relative to methanol alone or in combination with methanol (See Table 5).

To determine if different carbon sources also effect the PHB polymer size distribution, KB203 and KB287 were grown in 4 L flask and fed methanol, methanol and ethanol, or ethanol alone. The PHB from the resulting biomass was extracted and analyzed by GPC as above. Table 6 shows that KB287 had decreased average polymer length when grown in ethanol relative to methanol as a sole carbon source. KB203 had reduced average polymer length in the cofeed condition.

TABLE 2

| | | | | | | | % |
|---|---|---|---|---|---|---|---|
| Name | Genes | Locus | SEQ ID NO: | | | Source | PHB |
| pKB200A | lacZ | | 34 | 33 | | | 14.5 |
| pE4A | hbd | Mext_4730 | 34 | 21 | 22 | M. extorquens PA1 | 11.3 |
| pE5A | phaZ3 | Mext_3776 | 34 | 23 | 24 | M. extorquens PA1 | 10.1 |
| pE16A | phaP1 | Mext_0493 | 34 | 7 | 8 | M. extorquens PA1 | 13.1 |
| pE17A | phaP2 | Mext_2223 | 34 | 9 | 10 | M. extorquens PA1 | 6.2 |
| pE18A | phaP3 | Mext_2560 | 34 | 11 | 12 | M. extorquens PA1 | 9.0 |
| pE19A | phaZ1, depA | Mext_0594 | 34 | 13 | 14 | M. extorquens PA1 | 14.0 |
| pE20A | phaZ2, depB | Mext_4205 | 34 | 15 | 16 | M. extorquens PA1 | 13.9 |
| pE21A | phaZ1_Re | | 34 | 36 | 37 | R. eutropha H16 ATCC 17699 | 12.3 |
| pE22A | phaZ_Rp | | 34 | 38 | 28 | R. pickettii T1 | 6.8 |
| pE23A | phaZ_Ac | | 34 | 39 | 30 | Acidovorax sp. SA1 | 7.9 |
| pE24A | phaY1_Re | | 34 | 17 | 18 | R. eutropha H16 ATCC 17699 | 12.6 |
| pE25A | phaY2_Re | | 34 | 19 | 20 | R. eutropha H16 ATCC 17699 | |
| pE26A | phaY_Rp | | 34 | 40 | 26 | R. pickettii T1 | |
| pE27A | phaY_Ac | | 34 | 41 | 32 | Acidovorax sp. SA1 | 7.9 |
| pE28A | phaM_Re | | 34 | 42 | 43 | R. eutropha H16 ATCC 17699 | 13.1 |
| pE39A | phaZ7_Pl | | 34 | 44 | 45 | Paucimonas lemoignei | 6.0 |
| pKB200C | lacZ | | 34 | 33 | | | 15.9 |
| pE4C | hbd | Mext_4730 | 34 | 21 | 22 | M. extorquens PA1 | |
| pE5C | phaZ3 | Mext_3776 | 34 | 23 | 24 | M. extorquens PA1 | |
| pE16C | phaP1 | Mext_0493 | 34 | 7 | 8 | M. extorquens PA1 | |
| pE17C | phaP2 | Mext_2223 | 34 | 9 | 10 | M. extorquens PA1 | |
| pE18C | phaP3 | Mext_2560 | 34 | 11 | 12 | M. extorquens PA1 | |
| pE19C | phaZ1, depA | Mext_0594 | 34 | 13 | 14 | M. extorquens PA1 | 11.6 |
| pE20C | phaZ2, depB | Mext_4205 | 34 | 15 | 16 | M. extorquens PA1 | 11.7 |
| pE21C | phaZ1_Re | | 34 | 36 | 37 | R. eutropha H16 ATCC 17699 | 6.9 |
| pE22C | phaZ_Rp | | 34 | 38 | 28 | R. pickettii T1 | 13.4 |
| pE23C | phaZ_Ac | | 34 | 39 | 30 | Acidovorax sp. SA1 | 13.0 |
| pE24C | phaY1_Re | | 34 | 17 | 18 | R. eutropha H16 ATCC 17699 | |
| pE25C | phaY2_Re | | 34 | 19 | 20 | R. eutropha H16 ATCC 17699 | 11.4 |
| pE26C | phaY_Rp | | 34 | 40 | 26 | R. pickettii T1 | 13.4 |
| pE27C | phaY_Ac | | 34 | 41 | 32 | Acidovorax sp. SA1 | 13.3 |
| pE28C | phaM_Re | | 34 | 42 | 43 | R. eutropha H16 ATCC 17699 | |
| pE39C | phaZ7_Pl | | 34 | 44 | 45 | Paucimonas lemoignei | 11.8 |
| KB203 | | | | | | | 9.1 |
| KB262 | Δ2560 | | 11 | | | | 7.4 |
| KB323 | Δ2223 | | 9 | | | | 6.7 |
| KB324 | Δ0493, 2223 | | 7 | 9 | | | 1.3 |
| KB326 | Δ2560, 0493, 2223 | | 11 | 7 | 9 | | 0.3 |
| KB214 | Δ3093 | | 5 | | | | 0.0 |

TABLE 3

| | | | | | | | | Mn change | Mw change |
|---|---|---|---|---|---|---|---|---|---|
| | | | | | | | | (control/ | (control/ |
| Strain | Vessel | Plasmid | Genes | Mn | MW | MP | PD | new) | new) |
| KB203 | 4 L flask | pKB200C | lacZ | 56188 | 198826 | 111069 | 3.54 | Control | Control |
| KB203 | 4 L flask | pE19C | phaZ | 54867 | 221043 | 119288 | 4.03 | 0.98 | 1.11 |
| KB203 | 4 L flask | pE20C | phaZ | 51604 | 187394 | 109857 | 3.63 | 0.92 | 0.94 |
| KB203 | 4 L flask | pE21C | phaZ1_Re | 47973 | 173207 | 116101 | 3.61 | 0.85 | 0.87 |
| KB203 | 4 L flask | pE22C | phaZ_Rp | 21974 | 113484 | 43939 | 5.16 | 0.39 | 0.57 |
| KB203 | 4 L flask | pE23C | phaZ_Ac | 56938 | 192072 | 121813 | 3.37 | 1.01 | 0.97 |
| KB203 | 4 L flask | pKB200C | lacZ | 67465 | 244218 | 125938 | 3.62 | Control | Control |
| KB203 | 4 L flask | pE24C | phaY1_Re | 56152 | 226370 | 135883 | 4.03 | 0.83 | 0.93 |
| KB203 | 4 L flask | pE25C | phaY2_Re | 71994 | 241597 | 138676 | 3.36 | 1.07 | 0.99 |
| KB203 | 4 L flask | pE26C | phaY_Rp | 69329 | 237876 | 141094 | 3.43 | 1.03 | 0.97 |
| KB203 | 4 L flask | pE27C | phaY_Ac | 68786 | 249723 | 155563 | 3.63 | 1.02 | 1.02 |
| KB203 | 4 L flask | pE39C | phaZ1_Pl | 22561 | 177318 | 141018 | 7.86 | 0.33 | 0.73 |
| KB203 | 4 L flask | pKB200A | lacZ | 56262 | 261008 | 232988 | 4.64 | Control | Control |
| KB203 | 4 L flask | pE4A | hbd | 42028 | 133988 | 96215 | 3.19 | 0.75 | 0.51 |
| KB203 | 4 L flask | pE5A | phaZ3 | 41873 | 127723 | 90232 | 3.05 | 0.74 | 0.49 |
| KB203 | 4 L flask | pE17A | phaP2 | 41419 | 137666 | 92514 | 3.32 | 0.74 | 0.53 |
| KB203 | 4 L flask | pE18A | phaP3 | 47234 | 170446 | 98020 | 3.61 | 0.84 | 0.65 |
| KB203 | 4 L flask | pE19A | phaZ1 | 43427 | 128493 | 93684 | 2.96 | 0.77 | 0.49 |
| KB203 | 4 L flask | pE20A | phaZ2 | 40700 | 139110 | 98428 | 3.42 | 0.72 | 0.53 |
| KB203 | 4 L flask | pE21A | phaZ1_Re | 33834 | 138632 | 96451 | 4.10 | 0.60 | 0.53 |
| KB203 | 4 L flask | pE22A | phaZ_Rp | 31046 | 165912 | 109136 | 5.34 | 0.55 | 0.64 |
| KB203 | 4 L flask | pE23A | phaZ_Ac | 55767 | 231740 | 118696 | 4.16 | 0.99 | 0.89 |
| KB203 | 4 L flask | pE24A | phaY1_Re | 45073 | 140845 | 99824 | 3.12 | 0.80 | 0.54 |
| KB203 | 4 L flask | pE27A | phaY_Ac | 53386 | 200732 | 110909 | 3.76 | 0.95 | 0.77 |
| KB203 | 4 L flask | pE28A | phaM_Re | 53977 | 181442 | 102842 | 3.36 | 0.96 | 0.70 |
| KB203 | 4 L flask | pE39A | phaZ7_Pl | 25478 | 184178 | 118246 | 7.23 | 0.45 | 0.71 |
| KB203 | 4 L flask | pKB200A | lacZ | 58132 | 218630 | 106762 | 3.76 | Control | Control |
| KB203 | 4 L flask | | | 71601 | 248424 | 142228 | 3.07 | Control | Control |
| KB326 | 4 L flask | | | 52385 | 112813 | 21912 | 2.92 | Control | Control |
| KB203 | 4 L flask | pE16A | phaP1 | 73427 | 239674 | 143830 | 2.59 | 1.03 | 0.96 |
| KB203 | 4 L flask | pE17A | phaP2 | 69562 | 239955 | 136305 | 2.64 | 1.33 | 2.13 |
| KB326 | 4 L flask | pE17A | phaP2 | 14773 | 42298 | 8331 | 3.15 | 0.28 | 0.37 |
| KB326 | 4 L flask | pE18A | phaP3 | 40111 | 220309 | 347593 | 3.73 | 0.77 | 1.95 |
| KB203 | 4 L flask | | | 62031 | 218269 | 112686 | 3.52 | Control | Control |
| KB262 | 4 L flask | | Δ2560 | 46548 | 168032 | 101370 | 3.61 | 0.75 | 0.77 |
| KB323 | 4 L flask | | Δ2223 | 60990 | 220302 | 118576 | 3.61 | 0.98 | 1.01 |
| KB326 | 4 L flask | | Δ2560, 0493, | 17054 | 33151 | 9591 | 1.94 | 0.27 | 0.15 |
| | | | 2223 | | | | | | |
| KB203 | 1 L fermenter | | | 95390 | 321206 | 211327 | 3.37 | Control | Control |
| KB387 | 1 L fermenter | | | 39511 | 203618 | 112272 | 5.15 | 0.41 | 0.63 |
| KB203 | 1000 L ferm. | | | 96059 | 294457 | 206454 | 3.07 | Control | Control |
| KB324 | 1000 L ferm. | | Δ0493, 2223 | 30411 | 95898 | 127642 | 3.15 | 0.41 | 0.33 |
| Sigma | PHB | Catalog | #363502 | 97980 | 379647 | 331253 | 3.87 | | |

TABLE 4

| | 100% | 60% | Digestibility | Increase in |
|---|---|---|---|---|
| | H2SO4 | H2SO4 | Ratio | Digestibility |
| Strain | (mg PHB) | (mg PHB) | (60%/100%) | (X) |
| 203 | 1.0915 | 0.307 | 0.281 | Control |
| 387 | 0.1995 | 0.059 | 0.296 | 1.05 |
| 203 + pKB200A | 0.396 | 0.0725 | 0.183 | Control |
| 203 + pE22A | 0.583 | 0.1775 | 0.304 | 1.66 |
| 203 + pE39A | 0.334 | 0.113 | 0.338 | 1.85 |

TABLE 5

| Strain | Feed | % PHB | Strain | Feed | Feed | % PHB |
|---|---|---|---|---|---|---|
| 203 | M | 32.5 | 287 | M | M | 28.8 |
| 203 | E | 32.0 | 287 | E | E | 16.9 |
| 203 | M E | 27.6 | 287 | M E | M E | 40.0 |
| 203 | M G | 28.2 | 287 | M G | M G | 23.3 |
| 203 | E G | 35.2 | 287 | E G | E G | 15.5 |
| 203 | M E G | 22.3 | 287 | M E G | M E G | 30.5 |
| 203 | A | 30.7 | 287 | A | A | 29.4 |
| 203 | S | 8.4 | 287 | S | S | 15.6 |
| 203 | Ma | 19.0 | 287 | Ma | Ma | 16.7 |
| 203 | M F | 27.2 | 287 | M F | M F | 21.3 |
| 203 | M A | 33.2 | 287 | M A | M A | 30.9 |
| 203 | M S | 24.5 | 287 | M S | M S | 24.8 |
| 203 | M Ma | 20.6 | 287 | M Ma | M Ma | 19.3 |
| 203 | E F | 30.5 | 287 | E F | E F | 36.5 |
| 203 | E A | 28.2 | 287 | E A | E A | 19.6 |
| 203 | E S | 26.1 | 287 | E S | E S | 24.4 |
| 203 | E Ma | 26.8 | 287 | E Ma | E Ma | 29.3 |
| 203 | M E Ma | 22.1 | 287 | M E Ma | M E Ma | 22.3 |

TABLE 6

| | | Carbon | | | | | Mn change | Mw change |
|---|---|---|---|---|---|---|---|---|
| Strain | Vessel | source | Mn | MW | MP | PD | (control/new) | (control/new) |
| KB203 | 4 L flask | MeOH | 64548 | 213880 | 117629 | 3.31 | Control | Control |
| KB203 | 4 L flask | MeOH/EtOH | 54372 | 164693 | 112550 | 3.03 | 0.84 | 0.77 |
| KB203 | 4 L flask | EtOH | 80125 | 290648 | 502820 | 3.63 | 1.24 | 1.36 |
| KB287 | 4 L flask | MeOH | 60693 | 201532 | 119987 | 3.32 | Control | Control |
| KB287 | 4 L flask | MeOH/EtOH | 72490 | 202521 | 122129 | 2.79 | 1.19 | 1.00 |
| KB287 | 4 L flask | EtOH | 43735 | 126794 | 100444 | 2.90 | 0.72 | 0.63 |

Although the foregoing invention has been described in some detail by way of illustration and examples for purposes of clarity of understanding, it will be apparent to those skilled in the art that certain changes and modifications may be practiced without departing from the spirit and scope of the invention, which is delineated in the appended claims. Therefore, the description should not be construed as limiting the scope of the invention.

All publications, patents, and patent applications cited herein are hereby incorporated by reference in their entireties for all purposes and to the same extent as if each individual publication, patent, or patent application were specifically and individually indicated to be so incorporated by reference.

Amino Acid and Nucleotide Sequences

SEQ ID NO: 1
Description: PhaA
Mext_3469, A9W7T5, Beta-ketothiolase Length:
Type: DNA
Length: 1185
Organism: M. extorquens
>ATGGCAGCCAGTGAAGATATCGTCATTGTCGGTGCGGCGCGTACGCCCGTCGGATCGTTC
GCCGGTGCCTTCGGTTCCGTGCCGGCCCACGAACTCGGCGCCACGGCGATCAAGGCCGCA
CTGGAGCGCGCGGGCGTTTCGCCGGACGACGTGGACGAGGTGATCTTCGGCCAGGTGCTC
ACCGCTGCCGCCGGGCAGAACCCGGCCCGTCAGGCCGCCATCGCCGCAGGCATCCCCGAG
AAGGCGACCGCCTGGGGTCTCAATCAGGTCTGCGGCTCGGGCCTGCGCACCGTCGCGGTC
GGCATGCAGCAGATCGCCAACGGCGACGCCAAGGTGATCGTGGCCGGCGGCCAGGAGTCG
ATGTCGCTCAGCCCGCACGCCCAGTACCTGCGCGGCGGCCAGAAGATGGGCGATCTCAAG
CTCGTCGACACCATGATCAAGGACGGCCTGTGGGACGCCTTCAACGGCTACCACATGGGC
CAGACCGCCGAGAACGTCGCCCAGGCCTTCCAGCTCACCCGCGAGCAGCAGGACCAGTTC
GCGGTTCGCTCGCAGAACAAGGCCGAGGCCGCCCGCAAGGAAGGCCGCTTCAAGGAAGAG
ATCGTCCCCGTCACCGTGAAGGGCCGCAAGGGCGACACGGTCGTCGACACCGACGAGTAC
ATCCGCGACGGCGCCACCGTCGAGGCGATGGCCAAGCTCAAGCCCGCCTTCGCCAAGGAC
GGCACCGTGACCGCGGCCAACGCCTCGGGCCTCAACGACGGCGCCGCCGCGCTGGTGCTG
ATGTCGGCCTCCGAGGCCGAGCGCCGGGGCATCACGCCGCTCGCCCGGATCGTGTCCTGG
GCGACCGCCGGCGTCGATCCCAAGGTGATGGGCACGGGCCCGATCCCGGCCTCGCGCAAG
GCCCTGGAGAAGGCCGGCTGGAAGCCCGCCGACCTCGACCTGATCGAGGCGAACGAGGCT
TTCGCCGCTCAGGCGCTGGCCGTGAACAAGGACATGGGCTGGGACGACGAGAAGGTGAAC
GTCAATGGCGGCGCCATCGCCATCGGCCACCCGATCGGTGCCTCCGGCGCCCGCGTCCTC
ATCACCCTGCTGCACGAGCTGAAGCGCCGCGACGCCAAGAAGGGCCTCGCCACGCTCTGC
ATCGGCGGCGGCATGGGTGTCGCCATGTGTGTCGAGCGGGTCTGA
SEQ ID NO: 2
Description: PhaA
Alias: Mext_3469, A9W7T5, Beta-ketothiolase
Length: 394
Type: Protein
Organism: M. extorquens
>MAASEDIVIVGAARTPVGSFAGAFGSVPAHELGATAIKAALERAGVSPDDVDEVIFGQVLTAAAGQNPARQAA
IAAGIPEKATANGLNQVCGSGLRTVAVGMQQTANGDAKVIVAGGQESMSLSPHAQYLRGGQKMGDLKLVDTMIK
DGLWDAFNGYHMGQTAENVAQAFQLTREQQDQFAVRSQNKAEAARKEGRFKEEIVPVTVKGRKGDTVVDTDEYI
RDGATVEAMAKLKPAFAKDGTVTAANASGLNDGAAALVLMSASEAERRGITPLARIVSWATAGVDPKVMGTGPI
PASRKALEKAGWKPADLDLIEANEAFAAQALAVNKDMGWDDEKVNVNGGAIAIGHPIGASGARVLITLLHELKR
RDAKKGLATLCIGGGMGVAMCVERV
SEQ ID NO: 3
Description: PhaB
Alias: Mext_3470, A9W7T6, Acetoacetyl-CoA reductase
Length: 729
Type: DNA
Organism: M. extorquens
>ATGGCTCAGGAACGCGTCGCCCTCGTCACGGGCGGAACGCGCGGCATCGGCGCCGCGATC
TCCAAGCGCCTGAAGGACAAGGGCTACAAGGTCGCCGCCAATTACGGCGGAAACGATGAG
GCGGCCAACGCCTTCAAGGCCGAGACCGGCATCCCGGTGTTCAAGTTCGACGTCGGCGAT
CTCGCAAGCTGCGAGGCCGGCATCAAGGCGATCGAAGCCGAACTCGGCCCGATCGACGTC
CTCGTGAACAACGCCGGCATCACCCGCGACGGCGCCTTCCACAAGATGACCTTCGAGAAG
TGGCAGGCGGTGATACGCACCAACCTCGACTCGATGTTCACCTGCACCCGTCCGCTGATC
GAGGGAATGCGCTCGCGCAATTTCGGGCGCATCATCATCATCTCGTCGATCAACGGCCAG
AAGGGCCAGGCCGGCCAGACCAACTACTCCGCGGCCAAGGCCGGCGTGATCGGCTTCGCC
AAGGCACTGGCGCAGGAGAGCGCCTCGAAGGGCGTCACAGTGAACGTGGTGGCCCCCGGC
TACATCGCCACCGAGATGGTGATGGCGGTGCCGGAAGACATCCGTAACAAGATCATCTCG
ACGATCCCGACCGGCCGCCTCGGCGAGGCCGACGAGATCGCTCACGCGGTCGAGTACCTC
GCCAGCGACGAGGCCGGCTTCGTCAACGGCTCGACCCTCACCATCAACGGCGGTCAGCAC
TTCGTCTG
SEQ ID NO: 4
Description: PhaB
Alias: Mext_3470, A9W7T6, Acetoacetyl-CoA reductase
Length: 242
Type: Protein
Organism: M. extorquens
>MAQERVALVTGGTRGIGAAISKRLKDKGYKVAANYGGNDEAANAFKAETGIPVFKFDVGD
LASCEAGIKATEAELGPIDVLVNNAGITRDGAFHKMTFEKWQAVIRTNLDSMFTCTRPLI
EGMRSRNFGRIIIISSINGQKGQAGQTNYSAAKAGVIGFAKALAQESASKGVTVNVVAPG
YIATEMVMAVPEDIRNKIISTIPTGRLGEADETAHAVEYLASDEAGFVNGSTLTINGGQH FV
SEQ ID NO: 5
Description: PhaC
Alias: Mext_3093, Poly(R)-hydroxyalkanoic acid synthase, A9VX26
Length: 1818
Type: DNA
Organism: M. extorquens
>GTGGGCACCGAGCGGACGAACCCGGCAGCGCCGGATTTCGAGACCATCGCGCGCAACGCG
AATCAGCTCGCGGAGGTGTTCCGGCAATCGGCCGCCGCCTCGCTGAAGCCGTTCGAGCCG
GCGGGTCAGGGAGCCCTGCTCCCAGGCGCGAACCTCCAGGGCGCCAGCGAGATCGACGAG
ATGACCCGCACCCTCACGCGGGTCGCGGAGACATGGCTGAAGGATCCCGACAAGGCGCTT
CAGGCCCAGACCAAGCTCGGCCAGTCCTTCGCCGCGCTCTGGGCCTCGACCCTGACCCGG
ATGCAGGGAGCCGTCACCGAGCCGGTCGTCCAGCCCCCGCCCACGGACAAGCGCTTCGCC
CATGCCGATTGGAGCGCGAACCCGGTCTTCGACCTGATCAAGCAGAGCTACCTGCTCCTT
GGCCGCTGGGCCGAGGAGATGGTCGAGACGGCCGAAGGCATCGATGAGCACACCCGCCAC
AAGGCGGAGTTCTACCTGCGCCAGCTCCTCTCGGCCTACTCGCCCTCGAACTTCGTGATG
ACGAACCCCGAACTCCTGCGCCAGACGCTGGAGGAGGGGGGCGCCAACCTGATGCGCGGC
ATGAAGATGCTGCAGGAGGATCTGGAAGCCGGCGGCGGTCAGCTCCGGGTGCGGCAGACG
GACCTGTCCGCCTTCACCTTCGGCAAGGACGTGGCGGTGACCCCCGGCGAGGTCATCTTC
CGCAACGATCTGATGGAGTTGATCCAGTACGCGCCCACGACCGAGACGGTGCTGAAGCGT
CCGCTGTTGATCGTGCCGCCCTGGATCAACAAGTTCTACATCCTCGATCTCAACCCGCAG
AAGAGCCTCATCGGCTGGATGGTGTCTCAGGGGATCACGGTGTTCGTGATCTCCTGGGTG
AACCCGGACGAGCGCCACCGCGACAAGGACTTCGAGTCCTACATGCGGGAAGGCATCGAG
ACCGCCATCGACATGATCGGCGTGGCGACCGGCGAGACCGATGTCGCGGCGGCGGGCTAC
TGCGTCGGCGGCACGCTGCTCGCCGTCACGCTGGCCTACCAGGCGGCGACCGGCAACCGC
CGGATCAAGAGCGCCACCTTCCTCACCACGCAGGTCGATTTCACCCATGCGGGCGATCTC
AAGGTCTTCGCCGACGAGGGGCAGATCAAGGCGATAGAGGAGCGGATGGCCGAGCACGGC
TACCTGGAGGGCGCGCGCATGGCCAACGCCTTCAACATGCTCAGGCCCAACGACCTGATC
TGGTCCTACGTCGTCAACAACTACGTGCGCGGCAAGGCGCCGGCCGCCTTCGACCTGCTC
TACTGGAACGCGGACGCCACGCGGATGCCCGCGGCCAACCACTCGTTCTACCTGCGCAAC
TGCTACCTCAACAACACGCTCGCCAAGGGGCAGATGGTGCTCGGCAACGTGCGCCTCGAC
CTCAAGAAGGTGAAGGTGCCGGTCTTTAACCTCGCCACCCGCGAGGACCACATCGCCCCG
GCGCTCTCGGTCTTCGAAGGGTCGGCCAAGTTCGGCGGCAAGGTCGATTACGTGCTGGCG
GGCTCGGGCCACATCGCCGGCGTCGTCGCCCCGCCGGGCCCCAAGGCCAAATACGGCTTT
CGCACCGGTGGCCCGGCCCGCGGCCGGTTCGAGGATTGGGTCGCGGCGGCGACGGAGCAT
CAAGGCTCGTGGTGGCCCTACTGGTACAAGTGGCTCGAGGAGCAGGCGCCCGAGCGCGTG
CCCGCCCGCATTCCCGGAACGGGGGCCCTGCCTTCCCTGGCGCCGGCACCCGGCACCTAT
GTCCGCATGAAGGCGTGA
SEQ ID NO: 6
Description: PhaC
Alias: Mext_3093, Poly(R)-hydroxyalkanoic acid synthase, A9VX26
Length: 605
Type: Protein
Organism: M. extorquens
>MGTERTNPAAPDFETIARNANQLAEVFRQSAAASLKPFEPAGQGALLPGANLQGASEIDE
MTRTLTRVAETWLKDPDKALQAQTKLGQSFAALWASTLTRMQGAVTEPVVQPPPTDKRFA
HADWSANPVFDLIKQSYLLLGRWAEEMVETAEGIDEHTRHKAEFYLRQLLSAYSPSNFVM
TNPELLRQTLEEGGANLMRGMKMLQEDLEAGGGQLRVRQTDLSAFTFGKDVAVTPGEVIF
RNDLMELIQYAPTTETVLKRPLLIVPPWINKFYILDLNPQKSLIGNMVSQGITVFVISWV
NPDERHRDKDFESYMREGIETAIDMIGVATGETDVAAAGYCVGGTLLAVTLAYQAATGNR
RIKSATFLTTQVDFTHAGDLKVFADEGQIKAIEERMAEHGYLEGARMANAFNMLRPNDLI
WSYVVNNYVRGKAPAAFDLLYWNADATRMPAANHSFYLRNCYLNNTLAKGQMVLGNVRLD
LKKVKVPVFNLATREDHIAPALSVFEGSAKFGGKVDYVLAGSGHIAGVVAPPGPKAKYGF
RTGGPARGRFEDWVAAATEHQGSWWPYWYKWLEEQAPERVPARIPGTGALPSLAPAPGTY
VRMKA
SEQ ID NO: 7
Description: PhaP
Alias: Mext_0493, Phasin, A9W003
Length: 324
Type: DNA
Organism: M. extorquens
>ATGCGCGACTTTGCCGAAATGAGTGTCGAACAGGCGCGCGCTGCACTTATCGTGTTCATG
CAGAGCGCGCGTAAGGCTACCGAGAGCGTGCAGGCGCAGACGCGAGCCGCTGAGCTGCCT
GTCAGCGTCGCTTACGTGCGTGGTCTCGAGCTGTTCGAGAATAACCTTGCCGCAACCTTT
GATGTCGCGCAGAAGCTGGTGCGGACCAGCAGCCTGCAGGACGCGCTGCAGATCCAATCC
GAGTACGTGCACGCGCAGTTCGCTTCCCTGCAGAGCCAAGCGAAGGAACTCATTAGCGCG
GCTCAGCCTGCCAAGGCCGCCTGA
SEQ ID NO: 8
Description: PhaP
Alias: Mext_0493, Phasin, A9W003
Length: 107
Type: Protein
Organism: M. extorquens
>MRDFAEMSVEQARAALIVFMQSARKATESVQAQTRAAELPVSVAYVRGLELFENNLAATF
DVAQKLVRTSSLQDALQIQSEYVHAQFASLQSQAKELISAAQPAKAA
SEQ ID NO: 9
Description: PhaP
Alias: Mext_2223, Phasin, A9W4W2
Length: 357
Type: DNA
Organism: M. extorquens
>ATGAGCACCCAGAGCTTCGAGATCCCCGCCGAGTTCCGTGAGTTCGCCGACAAGAGCGTC
GATCAGGCTCGCAACGCCTTCGGCACCTTCCTGAACGGTGCGGTGAAGACCTCCGAGCAG
CTCCGGAACTCGGCCTCGACCGTCCAGTCGACGCTGAACGCGGCGGTGCTCAAGAGCCTC
GACCACACCAAGACCAACGCTGACGCTGCCTTCGACTACGTGCAGCGCGTCGTGCGCGCG
AAGGACCCGCGCGAGGCCTTCGAGATCCAGTCCGAGTTCCTGAAGACCCAGTTCGCCGCG
TTCCAGGCCCAGGCCAAGGAATACGGTGCCCTCGCTCAGAGCGCCGCCGGCCGCTAA
SEQ ID NO: 10
Description: PhaP
Alias: Mext_2223, Phasin, A9W4W2
Length: 118
Type: Protein
Organism: M. extorquens
>MSTQSFEIPAEFREFADKSVDQARNAFGTFLNGAVKTSEQLRNSASTVQSTLNAAVLKSL
DHTKTNADAAFDYVQRVVRAKDPREAFEIQSEFLKTQFAAFQAQAKEYGALAQSAAGR
SEQ ID NO: 11
Description: PhaP
Alias: Mext_2560, Phasin, A9W5U8
Length: 477
Type: DNA
Organism: M. extorquens
>GTGACAAACACTCCGAATTACGAAGTCCCGACCGAGATGCGCGACTTCGCCGAGAAGAGC
GTCGAGCAGGCCCGCAAAGCGTTCGATTCGTTCATCGGCGCAGCCCGCCGCACCGCCGAC
ACCGTCCAGGGCTCGACCGACCTCGCCCGCAGCAACGCCACCAGCATCTCCTCGCGCGGC
TTCGAGTACGCTGAGCAGAACGTCAACGCCGCGTTCGACCTGGCGCAGAAGCTCGTGCGC
TCGCGCGATGTCCAGGAGGCCATGCAGCACCAGGCGGAGTTCGTGCGCGCGCAGTTCGCG
GCGATTCAGGCGCAGGCCAAGGAGTTCGGTGGCCTCGCACAGAGCGCCTTTCAGCAGAGC
GCCGAGAACGCCAAGAGCGTCATGCAGCAGGGTGCCGCCGACGCTCGCCAAGCCTACGAG
CAGGGCGTCGAGACGGCCCGCGAGAATGCCAACGACGCGCAGAAGTCTTCTTCCTGA
SEQ ID NO: 12
Description: PhaP
Alias: Mext_2560, Phasin, A9W5U8
Length:
Type: Protein
Organism: M. extorquens
>MTNTPNYEVPTEMRDFAEKSVEQARKAFDSFIGAARRTADTVQGSTDLARSNATSISSRG
FEYAEQNVNAAFDLAQKLVRSRDVQEAMQHQAEFVRAQFAATQAQAKEFGGLAQSAFQQS
AENAKSVMQQGAADARQAYEQGVETARENANDAQKSSS
SEQ ID NO: 13
Description: PhaZ
Alias: Mext_0594, DepA, Intracellular PHB depolymerase, A9W0A2
Length: 1245
Type: DNA
Organism: M. extorquens
>ATGGACCTGGCATATATCTGGTACGAGACGGCCCGCGCGATGTTGACGCCGGCCCGGCTC
GCGGCGGATGCCGCTCGGCACAGCCTGGCGAATCCCGGCAATCCGTTCGCCTACGGTCCC
TATGCCCGCTCGGCGGCCGCCGCGCTGGAAATGTTCGAGCGTGTGACCCGCCGCTACGGC
AAGCCGGCCTTCGGCCTGCCGACGACCGTGATTGACGGCCAGGTCGTCCCGGTCTCCGAA
CGCGTCGTCTGGGAACGGCCCTTCGGCCGGGTGATCGCCTTCGATCGGGCGCTGCCCGCC
GGGCATTCGGAGCCGCAGCCCAAGCTCCTGATCGTGGCGCCCATGTCGGGCCATTACGCG
ACGCTCCTGCGCGGCACCGTCGAGGCGATGCTGCCCAACCACCGGGTCTTCATCACGGAT
TGGTCCGACGCGCGCATGGTCCCGTTGCTGGACGGGCGCTTCGACCTCGGCACGTATATC
GATTACCTGCAGGCGATGTTCCGTGACCTCGGGCCGGACCTGCACGTGATGGCCGTCTGC
CAGCCCGCTGTGCCGGTCTTCGCCGCGGTCGCGCTGATGGAGGCGGCGGATTCGGCCCAC
GTGCCGGTCTCGATGACCCTGATGGGCGGGCCGATCGACACCCGCCGCTCGCCGACCGCC
GTGAACTGCCTCGCGCAGGAGCGCGGCATGGCTTGGTTCGAGAAGAACTGCATCACGGTG
GTGCCGCCGCTCTATCCGGGGGCGATGCGCCGGGTCTATCCCGGCTTCCTGCAGCTTTCG
GGCTTCATGGCGATGAACCTCGATCGCCACGTCACCGCCCATACCGACATGTTCCATCAC
CTCGTGACCGGCGACGGCGATTCGGCGGAGAAGCACCGCGACTTCTACGACGAGTATCTC
GCGGTGATGGACCTGACGGCGGAATTCTACCTCCAGACCGTGCAGACCGTCTTCGTCGAT
CACGCCCTGCCGCGCGGGCGGATGCGCCATGACGGACGGTTGGTCGATCTCTCGGCGATC
CGCCGTTGCGCCATCCTCGCCGTCGAGGGCGAGAACGACGACATTTCCGGCGTCGGCCAG
ACCAAGGCCGCCCTCGATCTCACCCCGAACCTGCCCGATGCCCGCAAGGCCTACCACATG
CAGGAGAAGGTGGGGCATTACGGCGTGTTCAACGGCTCGCGCTTCCGGTCCGTCATCGCC
CCGCGCATCGCCCGCTTCGTGCGCGAGATGGAGGGAAGCGCCTGA
SEQ ID NO: 14
Description: PhaZ
Alias: Mext_0594, DepA, Intracellular PHB depolymerase, A9W0A2
Length: 414
Type: Protein
Organism: M. extorquens
>MDLAYIWYETARAMLTPARLAADAARHSLANPGNPFAYGPYARSAAAALEMFERVTRRYG
KPAFGLPTTVIDGQVVPVSERVVWERPFGRVIAFDRALPAGHSEPQPKLLIVAPMSGHYA
TLLRGTVEAMLPNHRVFITDWSDARMVPLLDGRFDLGTYIDYLQAMFRDLGPDLHVMAVC
QPAVPVFAAVALMEAADSAHVPVSMTLMGGPIDTRRSPTAVNCLAQERGMANFEKNCITV
VPPLYPGAMRRVYPGFLQLSGFMAMNLDRHVTAHTDMFHHLVTGDGDSAEKHRDFYDEYL
AVMDLTAEFYLQTVQTVFVDHALPRGRMRHDGRLVDLSAIRRCAILAVEGENDDISGVGQ
TKAALDLTPNLPDARKAYHMQEKVGHYGVFNGSRFRSVIAPRIARFVREMEGSA
SEQ ID NO: 15
Description: PhaZ
Alias: Mext_4205, DepB, PHB depolymerase, A9VY20
Length: 1365
Type: DNA
Organism: M. extorquens
>ATGCTGTATCCTCTTTACGAGGCCGGCCATCTTATGCTCGCACCGATGCGCTTGGCGGCG
GAAGCCACGAAGCTTGCCTGCGAAAACCCGTTCAATCCCTTCGCCTACGCGCCGCAGAGC
CGCACCATGGCCGCGGGCTGCGAGATGTTCGAGCGTGCCACCCGCGTCTACGCCAAGCCG
GCCTTCGGGCTCGGCGTGCCGGAGCGGGTGGTCTGGGAGCGCCCCTTCTGCCGCGTCGTG
GCCTTCGGCGAGCCCTCCGCGGAACTGGAGGCGAAGCCGAAGCTGCTGATCGTTGCGCCG
ATGTCGGGCCATTACGCCACGCTGCTGCGCGGCACGGTCGAGGCGTTCCTCCCCAGCCAT
CAGGTCTTCATCACCGATTGGTCCGACGCGCGTCAGGTGCCGGCGAGCGCCGGCCGGTTC
GGCCTCGACGATTACATCGATACCTGCATCGCCCTGTTCGCAGCACTCGGGCCGGACCTC
CACGTCGCGGCGGTTTGTCAGCCCTCGGTGCCGGTGCTTGCCGCCATCGCCCGCATGGAA
GCGGAGGATCACCCGCTCGTGCCGCGCTCGGCCGTGCTGATGGGCGGTCCCGTCGATACC
CGCCGCTCGCCGACCGCCGTCAACCTCATGGCCGAGGAGAAGGGCTTCGCGTGGTTCGAG
CGGCACTGCATCCACAGGGTGCCGGGCGGATATCCGGGAGCCGGCCGCGCGGTCTATCCG
GGCTTCCTTCAGCTCGCCGGCTTCATGGGGATGAACCTTGAGCGCCACCGGGACGCCCAC
CACGCGATGTTCGACCATCTCGTGCGCGGCGACGGCGACTCGGCCTCCCGCCATCGTGCC
TTCTACGACGAGTATCTCGCAGTCATGGACCTGACTGCCGAGTTCTATCTCGAGACGATT
GAGCGGGTCTTCATCAGCCACGACTTGCCCCGCGGTACCCTGCGCCATCGCGGCGAACGG
GTCGATCTCGGCGCGATCCGCCGCTGCCACCTGATGGCGGTGGAGGGCGAGAAGGACGAC
ATCACCGGCCTCGGCCAGACCAAGGCCGCGCTCGACCTCGCGGTAAACCTGCCCGAGGCG
GCCAAGACCTACCATATGCAGCCGGGAGCCGGGCATTACGGCATCTTCAACGGCTCGCGC
TTCCGCCAGGATATCGCGCCGTTGGTCTGCAGCTTCATGGAACGCAGCCTTCGACCGGCT
GCGCCGCGCCCGGCCCCGGTGGTGCCGGCACCGGAGCCGCACCCGATTATCCTGCGCCAC
GGCCCCATCATGCAGCCTCCGCGCGCCACGCCCGCCCGCGCCATCGTCTGGCCCGAGCCT
CTGGTCGATCGACCGGGAACGATCCCGCAGCGGATCGCACTGTAA
SEQ ID NO: 16
Description: PhaZ
Alias: Mext_4205, DepB, PHB depolymerase, A9VY20
Length: 454
Type: Protein
Organism: M. extorquens
>MLYPLYEAGHLMLAPMRLAAEATKLACENPFNPFAYAPQSRTMAAGCEMFERATRVYAKP
AFGLGVPERVVWERPFCRVVAFGEPSAELEAKPKLLIVAPMSGHYATLLRGTVEAFLPSH
QVFITDWSDARQVPASAGRFGLDDYIDTCIALFAALGPDLHVAAVCQPSVPVLAAIARME
AEDHPLVPRSAVLMGGPVDTRRSPTAVNLMAEEKGFAWFERHCIHRVPGGYPGAGRAVYP
GFLQLAGFMGMNLERHRDAHHAMFDHLVRGDGDSASRHRAFYDEYLAVMDLTAEFYLETI
ERVFISHDLPRGTLRHRGERVDLGAIRRCHLMAVEGEKDDITGLGQTKAALDLAVNLPEA
AKTYHMQPGAGHYGIFNGSRFRQDIAPLVCSFMERSLRPAAPRPAPVVPAPEPHPIILRH
GPIMQPPRATPARAIVWPEPLVDRPGTIPQRIAL
SEQ ID NO: 17
Description: PhaY1
Alias: PhaY1_Re, PhaZ2, Q0K9H3, PhaZb, Oligomer hydrolase, D-(-)-3-
hydroxybutyrate oligomer hydrolase
Length: 2274
Type: DNA
Organism: Ralstonia eutropha
>ATGGCCGCGCCAGCGGTTGCCGTGTTTCCGATTTCCGCAGTTCGCAGTCTCCGCAGTCCT
CGATCTGAAGACAATTCGACAGCCCCGGCCATCCGGGTGCGACAGGAGGAGGTTGACATG
CATTCCACGCAGATCCCGCCGCAGCAGAAACAGAAACGCCGCCTCAGGCTCACGGTGCTG
GCGGCCGCCGCATCGATGCTGGCAGCCGCCTGCGTCTCGGGCGATGACAACAACAACGGC
AACGGCAGCAACCCCAATACCAAGCCGGCGAATATCGGCACGGTCACGATCAACAGCTAC
AACGGCACCACCGACGACCTGCTCACTGCGGGCCTGGGCAAGGACGGCCTGGCCAGCGCC
ACCGCGCCACTGCCGGCCAATCCCACCGCGCCGACCGCGGCGGAGCTGCGGCGCTATGCG
ATCCATACCAACTATCGTGCCATCGTCGACACCACCGCCAGCGGCGGCTACGGCTCGCTC
TACGGCCCCAATGTCGACGCGCAGGGCAATGTCACCGGTTCCGACGGCAAGGTGGCCGGC
GTGGAGTACCTGGCCTTTTCGGACGATGGCTCGGGCCAGCAGAACGTGACCATGCTGGTG
CAGATTCCCGCGTCGTTCAACACCTCGAAGCCATGCATGATCACCGCTACCTCGTCCGGT
TCGCGCGGCGTCTATGGCGCAATCGCCACCGGCGAGTGGGGCCTGAAGCGCGGCTGCGCG
GTAGCCTATACCGACAAGGGCACCGGCGCCGCGCCGCATGACCTGGATACCGACACCGTG
CCGCTGATCGACGGCACCCGCGCCACGCGCGCGGCGGCCGGCAAGAACGCGCAGTTTGCC
GCGCCGGCGGGGGCCACCTCGCTGGCGGACTTCACCGCTGCCAACCCGCACCGGCTGGCG
TTCAAGCACGCGCATTCACAGCGCAACCCGGAGAAGGACTGGGGCAAGTTCACGCTGCAG
GCGGTGGAATTTGCGATCTGGGCGATCAATGACCGCTTCGGCGCCGTGTCGGCCAACGGC
ACCCGCCAGCGCACCCTGGACAAGGACAGGATCGTGGTGATCGCGTCCAGCGTGTCCAAC
GGCGGCGGTGCCGCGGTGGCGGCGGCCGAGCAGGATGCCGGCGGGCTGATCGACGGCGTG
GCGGTGGGCGAGCCCAACCTGAACATGCCGCCCAATACCGGCATCGTGGTGCAACGCGGC
GCGACGCCGGTGGCAGCTTCGGGCCGCACGCTGTACGACTACACCACCACGGCCAACCTG
CTGCAGCACTGCGCCGCGCGGGCCACCGCGCTGACCCAGGCGCCGTTCTACACCAACCCG
GCCACGGCGACGTTCTTTGCCAACCGCTGCCAGACGCTGGCGGAAAAGGGGCTGGTGAGC
GGCGCGAACACGGACGAACAGAGCGCCAGCGCGCTGCAGGCGCTGCATGACGCCGGCTGG
GAAGCGGAATCGGACGATCTGCACCCGTCGCTGGCCGTGTTCGACGTGGCCGCCGCGATC
TCGGTCAACTATGCCAACGCCTATGCGCAGGCCAGCGTCACCGACCGGCTGTGCGGCTAC
AGCTTTGCCAGCACGCTGACCGACCTGAAGCCCGCGGCAATCGCACCCGCGGCGCTGGCG
TCGATGTTCGCCACCGGCAACGGCGTGCCGCCGCAGCCGCCGGTCCAGCTGATCAATGAC
CTCGATCCGCAGCATGGCCCGTACCTGAACCTGGCGTCTGTTTCACCGTCGACGCTGCGT
GAAGACCTGAACTACGACGGCGCCAACTGCCTGCGCAGCCTGCTGGCCGGCTCCGACGCC
GCGGCACGCGCCTTGCAGGCCGGCCAGGCGCTGACGCTGCGCAACGGCAACCTGCGCGGC
AAGCCGGCGGTGATCGTGCACGGGCGCAGCGACGGGCTGCTGCCGGTCAACCATACCTCG
CGTCCGTATCTCGGCCTGAACCGGCAGCAGGAGGGCGTGACCAGCAAGCTGTCATACGTG
GAGGTCGAGAACGCCCAGCACTTCGATGCCTTCATCGGCCTGGTGCCGGGCTACAGCAAC
CGCTATGTGCCGCTGCACGTCTACCTGAACCGGGCCCTGGATGCGGTCTACGACAACCTC
ACCGCGGGCAAGGCGCTGCCGCCGTCGCAGGTGCTGCGCACCACGCCGCGCGGCGGCACC
CTCAACACGCCCGCGCCGGCGCTGCTGCCGTCCAACGTGCCGCCGTTCGCCGCGTCGCCT
GCGGCCGGCAACGCGATTACCGTCAACGCTAATGCCGTACAGGTGCCTGACTGA
SEQ ID NO: 18
Description: PhaY1
Alias: PhaY1_Re, PhaZ2, Q0K9H3, PhaZb, Oligomer hydrolase, D-(-)-3-
hydroxybutyrate oligomer hydrolase
Length: 718
Type: Protein
Organism: Ralstonia eutropha
>MHSTQIPPQQKQKRRLRLTVLAAAASMLAAACVSGDDNNNGNGSNPNTKPANIGTVTINS
YNGTTDDLLTAGLGKDGLASATAPLPANPTAPTAAELRRYAIHTNYRAIVDTTASGGYGS
LYGPNVDAQGNVTGSDGKVAGVEYLAFSDDGSGQQNVTMLVQIPASFNTSKPCMITATSS
GSRGVYGAIATGENGLKRGCAVAYTDKGTGAAPHDLDTDTVPLIDGTRATRAAAGKNAQF
AAPAGATSLADFTAANPHRLAFKHAHSQRNPEKDWGKFTLQAVEFAIWAINDRFGAVSAN
GTRQRTLDKDRIVVIASSVSNGGGAAVAAAEQDAGGLIDGVAVGEPNLNMPPNTGIVVQR
GATPVAASGRTLYDYTTTANLLQHCAARATALTQAPFYTNPATATFFANRCQTLAEKGLV
SGANTDEQSASALQALHDAGWEAESDDLHPSLAVFDVAAAISVNYANAYAQASVTDRLCG
YSFASTLTDLKPAATAPAALASMFATGNGVPPQPPVQLINDLDPQHGPYLNLASVSPSTL
REDLNYDGANCLRSLLAGSDAAARALQAGQALTLRNGNLRGKPAVIVHGRSDGLLPVNHT
SRPYLGLNRQQEGVTSKLSYVEVENAQHFDAFIGLVPGYSNRYVPLHVYLNRALDAVYDN
LTAGKALPPSQVLRTTPRGGTLNTPAPALLPSNVPPFAASPAAGNAITVNANAVQVPD
SEQ ID NO: 19
Description: PhaY2
Alias: PhaY2_Re, Q0KBZ6, PhaZc, Oligomer hydrolase, D-(-)-3-hydroxybutyrate
oligomer hydrolase
Length: 882
Type: DNA
Organism: Ralstonia eutropha
>ATGTCTGCCAGTCCGCGTCTCGGTTTTGTCCAGTGCATCAGTCCGGCGGGCCTGCACCGC
ATGGCCTACCACGAGTGGGGCGACCCCGCCAATCCGCGCGTGCTGGTGTGCGCGCATGGG
CTGACGCGCACCGGCCGCGACTTCGACACCGTCGCCAGCGCGCTGTGCGGCGACTACCGC
GTGGTCTGCCCCGATGTGGCCGGGCGCGGCCGCTCGGAATGGCTGGCCGATGCCAACGGC
TACGTGGTGCCGCAGTATGTGTCCGACATGGTCACGCTGATTGCGCGGCTCAACGTGGAG
AAGGTGGACTGGTTCGGCACCTCGATGGGCGGGCTGATCGGCATGGGCCTGGCCGGGCTG
CCGAAGTCGCCGGTGCGCAAGCTGCTGCTCAACGACGTGGGCCCGAAGCTGGCGCCGTCG
GCGGTGGAACGGATCGGCGCCTACCTGGGGCTGCCGGTGCGCTTCAAGACCTTCGAGGAA
GGCCTGGCCTACCTGCAAACCATCAGCGCATCGTTCGGCCGCCATACGCCCGAGCAGTGG
CGCGAGCTCAACGCCGCCATCCTGAAACCGGTGCAGGGCACGGACGGCCTGGAATGGGGC
TTGCATTACGATCCGCAGCTGGCGGTGCCGTTCCGCAAATCCACGCCCGAGGCCATTGCT
GCCGGCGAGGCCGCGCTCTGGCGCAGCTTCGAGGCCATCGAAGGCCCGGTGCTGGTGGTG
CGCGGCGCGCAGTCGGACCTGCTGCTGCGCGAGACCGTGGCCGAAATGGTGGCGCGCGGC
AAGCATGTGAGTTCGGTGGAAGTGCCCGACGTGGGCCATGCCCCGACCTTTGTCGATCCG
GCGCAGATTGCGATCGCCCCGCAGTTCTTTACCGGGGCCTGA
SEQ ID NO: 20
Description: PhaY2
Alias: PhaY2_Re, Q0KBZ6, PhaZc, Oligomer hydrolase, D-(-)-3-hydroxybutyrate
oligomer hydrolase
Length: 293
Type: Protein
Organism: Ralstonia eutropha
>MSASPRLGFVQCISPAGLHRMAYHEWGDPANPRVLVCAHGLTRTGRDFDTVASALCGDYR
VVCPDVAGRGRSEWLADANGYVVPQYVSDMVTLIARLNVEKVDWFGTSMGGLIGMGLAGL
PKSPVRKLLLNDVGPKLAPSAVERIGAYLGLPVRFKTFEEGLAYLQTISASFGRHTPEQW
RELNAAILKPVQGTDGLEWGLHYDPQLAVPFRKSTPEAIAAGEAALWRSFEATEGPVLVV
RGAQSDLLLRETVAEMVARGKHVSSVEVPDVGHAPTFVDPAQTAIAPQFFTGA
SEQ ID NO: 21
Description: Hbd
Alias: Mext_4730, 3-hydroxybutyrate dehydrogenase, A9W959
Length: 786
Type: DNA
Organism: M. extorquens
>ATGAGCCTGCAAGGGAAAGCCGCCGTGGTCACCGGCTCGACGAGCGGCATCGGCCTCGCC
ATCGCCAAGAGCTTCGCGAAAGACGGCGCGAACGTGGTCCTCAACGGATTCGGCAACCCC
GAAGACATCGAGCGGACCCGCAGCGGCATCGAGAGCGAGTTCGGGGTCAAGGCGGTCTAT
TCGCCCGCCGACCTCACCAAGCCGGACGAGATCGGCGGGCTGATCGCACTCTCGGTCGAG
ACGTTCGGCAGCATCGACATCCTCGTGAACAATGCGGGCATCCAGTACGTCTCGCCGATC
GAGGACTTTCCGGTCGAGAAGTGGGACCAGATCATCGCGCTCAACCTCTGCTCGGCCTTT
CATACGCTGCGAGCGGCCGTGCCGCACATGAAGGCGAAGGGCTGGGGCCGGGTCATCAAC
ACGGCCTCGGCGCACTCGATGGTCGCCTCGCCCTACAAGTCGGCCTACGTCGCGGCCAAG
CACGGCGTCGTCGGCCTCACCAAGACGGCGGCGCTCGAACTCGCCACCCACGGCATCACC
GTGAACTGCATCTCACCGGGCTATGTCTGGACGCCGCTGGTGGAAAGCCAGATCCCGGAC
ACGATGAAGGCGCGCGGCCTCACCAAGGAGCAGGTGATCGAGGAGGTGCTGCTCAAGGCG
CAGCCGACCAAGGAATTCGTGACGATCGATCAGGTGGCCGCGCTCGCCCTGTTCCTGTGC
ACGGACAGCGCCAGCCAGATCACCGGTGCCAACATCGCCATGGATGGCGGCTGGACGGCG CAGTAG
SEQ ID NO: 22
Description: Hbd
Alias: Mext_4730, 3-hydroxybutyrate dehydrogenase, A9W959
Length: 261
Type: Protein
Organism: M. extorquens
>MSLQGKAAVVTGSTSGIGLAIAKSFAKDGANVVLNGFGNPEDIERTRSGIESEFGVKAVY
SPADLTKPDEIGGLIALSVETFGSIDILVNNAGIQYVSPIEDFPVEKWDQIIALNLCSAF
HTLRAAVPHMKAKGWGRVINTASAHSMVASPYKSAYVAAKHGVVGLTKTAALELATHGIT
VNCISPGYVWTPLVESQIPDTMKARGLTKEQVIEEVLLKAQPTKEFVTIDQVAALALFLC
TDSASQITGANIAMDGGWTAQ
SEQ ID NO: 23
Description: PhaZ
Alias: Mext_3776, polyhydroxyalkanoate depolymerase, A9VXH8
Length: 1215
Type: DNA
Organism: M. extorquens
>ATGCTCTACCAAGCCCTCGATGTCCAATCGGACATCGCCCGGCAGACCCGCCAATGGGGC
CGCCTGCTGCAGGAAGCCTCCGCGCCGTGGATGCGGACGCCCTGGCACGACGCCGCGAAA
TGGTGGTCGGCGGGCGCGCGCATGATGATGCGCGCCGGCCTCACCTTCGCGCGGCCGGCC
TACGGCATCCACGCCGTCATGGTCGGCAACCGCGAAGTGCCGGTGATCGAGGAGCCGGTG
CTCGCCACGCCCTTCGGCACGCTGCTCCGCTTCCGCAAGGACATCGACACCGTCCAGCCC
AAGGTGCTGGTGCTCGCCCCCCTCTCGGGCCACTTCGCCACGCTGCTGCGCAGCACCGTG
CGCACGCTGCTGCCCGACCACGACGTCTACATCACCGACTGGCACAACGCCCGCGACGTG
CCGCTCTCGGAAGGGCGGTTCGGCTTCGACGACTACGTCGATCACGTGGTGCGCTTTCTG
GAGACCATCGGCGAGGGCGCCCACCTCATGGCCGTGTGCCAGCCCGCGGTGCAGGCGCTC
GCGGCCACGGCGCTGATGGCGCACACCAAGAATCCGGCGCAGCCGCGCAGCATGACCCTG
ATGGCCGGACCGGTCGATTGCCGCGTCAGCCCGACCTCGGTGAACCGGCTCGCCGTCTCG
AAGCCGATCGAGTGGTTCGAGAAGAACCTGATCGAGACGGTGACCGGACGCCACAAGGGG
GCGGGGCGGCGGGTCTATCCCGGCTTCACGCAGGTCTCCGCCTTCGTCTCGATGAATGCC
AAGCGCCACCGGGACGCGCATACGGACCTGTTCTGGCACTACGTCGACGGCAGCGCCGAC
AAGGCGCAGGCGATCGAGACCTTCTACGACGAGTATTTCGCCGTCCTCGACCTCGCCGCC
GAGTTCTACCTCGAGACGGTCAAGATCGTCTTCCAGGACTACACCCTGGCCCGCAACCAG
CTCACCTATCGCGGCGAGCCCATCGATATGGGCGCGATCCGGCGCACCGCCCTGATGACG
GTGGAAGGCGAGCGCGACGACATCTGCGCCGTGGGCCAGACCATGGCCGCCCACGACCTC
TGCTCGAGCCTGCCGCCGCACATGAAGACCCACCACCTCCAAACCGGCGTGGGCCACTAC
GGCGTGTTCTCGGGCCGGAAGTGGGAGGCGCAGACCTATCCGCTCGTGCGCAACTTCATC
GCCTCGCACGCCTGA
SEQ ID NO: 24
Description: PhaZ
Alias: Mext_3776, polyhydroxyalkanoate depolymerase, A9VXH8
Length: 404
Type: Protein
Organism: M. extorquens
>MLYQALDVQSDIARQTRQWGRLLQEASAPWMRTPWHDAAKWWSAGARMMMRAGLTFARPA
YGIHAVMVGNREVPVIEEPVLATPFGTLLRFRKDIDTVQPKVLVLAPLSGHFATLLRSTV
RTLLPDHDVYITDWHNARDVPLSEGRFGFDDYVDHVVRFLETIGEGAHLMAVCQPAVQAL
AATALMAHTKNPAQPRSMTLMAGPVDCRVSPTSVNRLAVSKPIEWFEKNLIETVTGRHKG
AGRRVYPGFTQVSAFVSMNAKRHRDAHTDLFWHYVDGSADKAQATETFYDEYFAVLDLAA
EFYLETVKIVFQDYTLARNQLTYRGEPIDMGAIRRTALMTVEGERDDICAVGQTMAAHDL
CSSLPPHMKTHHLQTGVGHYGVFSGRKWEAQTYPLVRNFIASHA
SEQ ID NO: 25
Description: PhaY
Alias: PhaY_Rp, D-(-)-3-hydroxybutyrate oligomer hydrolase, Extracellular
3HB-oligomer hydrolase, e3HBOH, Q9X6X9
Length: 2226
Type: DNA
Organism: Ralstonia pickettii
>ATGAAAACGATACAAGGGAAGAGTCCGGGCCGCTGGTATTCGCGCGGCATGCTGCTGGCA
GCGATGGCGGCGTCCGGCGTCATCGGCCTGGCCGCGTGCGGTGGCGGCAATGATGGCAAC
TCAGCAGGCAACAATGGCAATGCCGGAGGCAACGGCAACAACAACGGCAACAACAACGGC
AATACGGTGAGCAACACCAAGCCGTCCTTCGTCGGTACCGTGACGGTCAGGCGATTCGAC
GGTGTGAGCGACGACTTGCTGACCGCGGGCCTGGGCGCCTCCGGGCTGGCTTCGGCCACG
GCACCTGCCGTGGCCAACGCCGTTGCGCCGACCGCGGCAGAGTTGCGGCGCCTGACCATC
TACAACAACTATCGCGCCTTGATCGACACCAGCGCCAAGGGTGGCTACGGCACACTCTAC
GGCCCCAACGTCGATGCCGACGGCAACGTCACCTCCGGCAATGGCATGGTGGCCGGCGCG
GAGTATGTCGCGTACCCGGATGACGGCTCCGGCCAGCAGAACGTGGTGCTGCTGGTGCAG
ATTCCCGACGCATTCGATGCCGCGCATCCGTGCATCATCACCGCGACCTCGTCGGGTTCG
CGCGGCATCTACGGGGCCATTTCGACCGGTGAGTGGGGACTCAAGCGCAAGTGCGCGGTC
GCCTATACCGATAAGGGTACCGGCGCCGGCCCGCACGACCTGGCCACCGACACGGTGCCG
CTGCAGGACGGCACGCGCACGACACGCACGCTCGCCGGCAACACGGCGCAATTCGCCGCG
CCGCTCGCCGCGAGCCGGCTTGCCGCCTTCAACGTGGCAACGCCCAACCGGCTGGCGTTC
AAGCATGCGCACTCGCAGCGCAACCCCGAGAAGGACTGGGGCCTCTTCACGCTGCAGGCG
GTGCAGTTCGCCTTCTGGGCCATCAACGACAAGCTGGGCATCTCCAGCGGGCAGACCGTC
AGCCAGTTGCCGGTGCGTCCCGGCAACACCATCGTGATCGCTTCCAGTGTGTCCAATGGT
GGCGGCGCGGCGATCGCGGCGGCCGAGCAGGACACCGGCAACCTGATCGATGGCGTGGCG
GTCGGCGAGCCCGCATTGAGCCTGCCGTCCTCGATCAACGTGCAGGTCAAGCGCGGCGGC
GCAAGCTTGCCGATCAACGGCAAGCCGCTGTTCGACTACGTCAGCTATGCCAACGAATTC
CGGCTGTGCGCGGCGCTGTCGGCCAGCGTGGCAAGCGCGCCGACGCAGGCTTACTTTGGA
GCGGCTTTAGGCTGGCCCGCCAGCGTGCAGGCGAACCGCTGCGCAGCGCTGCACGCCAAG
GGCCTGTTGTCGTCCACCACCACGGCAGCACAGGCCGACGAGGCGCTGCAGAAGATGCGC
GACTACGGTTGGGAGCCCGAATCCGACCTCCTGCATGCCTCCATGGCGTACTTCGAGATC
GATCCGTCGGTCGCCACCACCTTCGGCAACGCCCTGGCGCGCGCCAGCGTGTTCGACAAT
CTGTGCGACCTCAGCTTTGCGGCGGTGGATGGCTCGTTCCACCCGGCCACGATGAACGCC
ACGGTGCTGGCGCAACTGGCCGCCACCGGCAACGGAGTTCCTCCCACGACCGGCGTGCAG
TTGATCAACAATATTGCCCAGGGTGGTGCGGCGCAGAGCAGGCAGTCGATCGACTCCTCC
GGTACGCAGGCCGCCAACCTGGATGGCGCGCTATGCCTGCGCAACCTGCTGAGCGGCAGC
GACGCCGCCTCGCAGGCGCTGCAGCTTGGCCTGTCGCAGACGCTGCGCAGCGGCAATCTG
CGCGGCAAGCCAGCCCTGATCGTGCAAGGGCGGAACGATGCCCTGCTGCCGGTCAACCAT
GGCGCTCGCCCGTATCTGGGCCTCAATGCGCAGGTCGATGGGAGCAGCAAGCTGTCGTAT
ATCGAGGTCACGAACGCCCAGCACTTCGATGGCTTCATTGATCTGTTGCCGGGATACGAC
TCGCTCTTCGTGCCCTTGGCCGTCTATGAGCAACGCGCGCTTGACGCCGTGTACGCGAAC
CTGAGGAGCGGCACGCCGCTGCCACCGTCGCAAGTGGTACGCACGACGCCGCGCGGCGGT
GCGGCAGGAGCGGCGCCGCCTATCACGGCCGCCAACGTGCCGAACTTCACCATGACTCCG
GCTGCGGGTGACCGTATCCAGGTGAGCGTCTCGGGTGGCGTGGCGACGGTTTCCGTGCCG AACTGA
SEQ ID NO: 26
Description: PhaY
Alias: PhaY_Rp, D-(-)-3-hydroxybutyrate oligomer hydrolase, Extracellular
3HB-oligomer hydrolase, e3HBOH, Q9X6X9
Length: 741
Type: Protein
Organism: Ralstonia pickettii
>MKTIQGKSPGRWYSRGMLLAAMAASGVIGLAACGGGNDGNSAGNNGNAGGNGNNNGNNNG
NTVSNTKPSFVGTVTVRRFDGVSDDLLTAGLGASGLASATAPAVANAVAPTAAELRRLTI
YNNYRALIDTSAKGGYGTLYGPNVDADGNVTSGNGMVAGAEYVAYPDDGSGQQNVVLLVQ
IPDAFDAAHPCIITATSSGSRGIYGAISTGEWGLKRKCAVAYTDKGTGAGPHDLATDTVP
LQDGTRTTRTLAGNTAQFAAPLAASRLAAFNVATPNRLAFKHAHSQRNPEKDWGLFTLQA
VQFAFWAINDKLGISSGQTVSQLPVRPGNTIVIASSVSNGGGAAIAAAEQDTGNLIDGVA
VGEPALSLPSSINVQVKRGGASLPINGKPLFDYVSYANEFRLCAALSASVASAPTQAYFG
AALGWPASVQANRCAALHAKGLLSSTTTAAQADEALQKMRDYGWEPESDLLHASMAYFEI
DPSVATTFGNALARASVFDNLCDLSFAAVDGSFHPATMNATVLAQLAATGNGVPPTTGVQ
LINNIAQGGAAQSRQSIDSSGTQAANLDGALCLRNLLSGSDAASQALQLGLSQTLRSGNL
RGKPALIVQGRNDALLPVNHGARPYLGLNAQVDGSSKLSYIEVTNAQHFDGFIDLLPGYD
SLFVPLAVYEQRALDAVYANLRSGTPLPPSQVVRTTPRGGAAGAAPPITAANVPNFTMTP
AAGDRIQVSVSGGVATVSVPN
SEQ ID NO: 27
Description: PhaZ
Alias: PhaZ_Rp, P12625, ePHBDP, Extracellular Poly (3-hydroxybutyrate)
depolymerase
Length: 1467
Type: DNA
Organism: Ralstonia pickettii
>ATGGTGAGAAGACTGTGGCGACGGATCGCAGGCTGGCTGGCGGCCTGCGTGGCGATCTTG
TGCGCGTTTCCATTGCATGCCGCCACGGCGGGGCCCGGTGCCTGGAGCAGCCAGCAGACC
TGGGCGGCGGACTCCGTCAATGGCGGTAACCTGACGGGCTACTTCTACTGGCCGGCCAGC
CAGCCGACCACGCCGAATGGCAAGCGCGCGCTCGTCCTGGTGCTGCACGGGTGCGTGCAG
ACGGCCTCGGGCGACGTCATCGACAACGCCAATGGCGCCGGCTTCAACTGGAAGTCGGTC
GCCGACCAGTATGGCGCCGTGATCCTGGCCCCGAATGCCACAGGGAACGTCTACAGCAAC
CATTGCTGGGACTACGCAAACGCCTCGCCCAGCCGCACGGCCGGTCACGTCGGCGTCCTG
CTGGACCTGGTCAATCGCTTCGTCACCAATTCGCAGTACGCCATCGACCCCAACCAGGTC
TACGTCGCCGGCTTGTCCTCCGGCGGCGGCATGACCATGGTGCTGGGCTGCATCGCGCCG
GACATCTTCGCCGGCATCGGCATCAACGCTGGTCCGCCGCCGGGCACCACCACCGCGCAG
ATCGGCTACGTGCCGTCAGGCTTCACGGCGACGACGGCCGCGAACAAATGCAACGCGTGG
GCAGGCTCCAACGCGGGCAAGTTCTCCACGCAGATCGCCGGTGCGGTCTGGGGAACCTCG
GACTACACCGTGGCGCAGGCGTATGGCCCGATGGATGCGGCGGCCATGCGTCTCGTCTAC
GGCGGCAACTTCACGCAGGGTTCGCAGGTGTCGATTTCGGGCGGCGGCACCAATACGCCG
TACACCGACAGCAACGGCAAGGTGCGCACCCATGAGATCTCGGTCTCCGGCATGGCCCAC
GCGTGGCCGGCCGGCACCGGCGGCGACAACACCAACTATGTCGATGCCACCCACATCAAC
TATCCGGTCTTCGTCATGGACTACTGGGTCAAGAACAACCTGCGCGCCGGCAGCGGGACG
GGGCAGGCAGGCAGCGCGCCGACCGGGCTTGCCGTCACGGCAACGACCTCCACGTCGGTC
TCGCTGTCGTGGAATGCCGTCGCCAATGCCAGCAGCTATGGCGTCTACCGCAACGGCAGC
AAGGTCGGTTCGGCGACGGCCACCGCTTATACCGATTCCGGCCTGATCGCCGGCACGACC
TACAGCTACACGGTGACCGCGGTCGATCCGACGGCAGGCGAAAGCCAACCCTCCGCCGCC
GTATCGGCGACGACGAAATCGGCCTTCACCTGTACTGCCACCACGGCCAGCAACTACGCG
CACGTGCAGGCCGGGCGCGCGCACGACAGTGGCGGCATTGCCTACGCGAACGGCTCGAAC
CAGAGCATGGGGCTCGACAACCTCTTCTACACGAGCACGCTGGCGCAGACGGCCGCCGGC
TACTACATCGTCGGCAATTGTCCATGA
SEQ ID NO: 28
Description: PhaZ
Alias: PhaZ_Rp, P12625, ePHBDP, Extracellular Poly (3-hydroxybutyrate)
depolymerase
Length: 488
Type: Protein
Organism: Ralstonia pickettii
>MVRRLWRRIAGWLAACVAILCAFPLHAATAGPGAWSSQQTWAADSVNGGNLTGYFYWPAS
QPTTPNGKRALVLVLHGCVQTASGDVIDNANGAGFNWKSVADQYGAVILAPNATGNVYSN
HCWDYANASPSRTAGHVGVLLDLVNRFVTNSQYAIDPNQVYVAGLSSGGGMTMVLGCIAP
DIFAGIGINAGPPPGTTTAQIGYVPSGFTATTAANKCNAWAGSNAGKFSTQIAGAVWGTS
DYTVAQAYGPMDAAAMRLVYGGNFTQGSQVSISGGGTNTPYTDSNGKVRTHEISVSGMAH
AWPAGTGGDNTNYVDATHINYPVFVMDYWVKNNLRAGSGTGQAGSAPTGLAVTATTSTSV
SLSWNAVANASSYGVYRNGSKVGSATATAYTDSGLIAGTTYSYTVTAVDPTAGESQPSAA
VSATTKSAFTCTATTASNYAHVQAGRAHDSGGIAYANGSNQSMGLDNLFYTSTLAQTAAG YYIVGNCP
SEQ ID NO: 29
Description: PhaZ
Alias: PhaZ_Ac, Q767A0, D(-)-3-hydroxybutyrate dehydrogenase, bdh
Length: 777
Type: DNA
Organism: Acidovorax sp. SA1
>ATGCTGAAAGGCAAAACTGCCCTCGTCACCGGCTCCACCAGCGGCATTGGTCTTGGCATC
GCCAAGGCCCTGGCGCGCCAGGGCGCCAACATCGTGCTCAACGGCTTTGGCGATGTGGAT
GGCCCGCGTGCCGAAGTGCTGGCCGCCGGTGAGGCCGCGGGGGCCCAGGTGGCGTACCAC
GGCGCGGACATGAGCCGTGTGCCACATCGAGACATGATGAAGTACAGCGCCAGCCAGTTC
GGCCGCGTGGACATCCTGGTCAACAACGCAGGCATCCAGCATGTGGCCAACGTGGAGAAC
TTCCCCGTGGAGCGCTGGGATTCCATCATCGCCATCAACCTGACCAGCGCCTTGCACACC
TCGCGCCTGGCCCTGCCCGCGATGAAGAGCGCCAACTGGGGCCGCATCATCAATGTGGCG
TCGGTGCACGGCCTGGTGGGATCAGCCCAGAAGTCCGCCTATGTGGCGGCCAAGCACGGC
ATCGTGGGCCTGACCAAGGTCACTGCGCTGGAAAACGCCACCACCGGCGTGACCTGCAAT
GCCATCTGCCCCGGCTGGGGTCTGACGCCACTGGTGCAAAAGCAGGTGGATGCCAAGGCC
GCAGAACATGGCATCTCGAACGAAGAGGCCAAGAAGCTGTTGCTGGGCGAAAAGGAGCCT
TCCATGCAGTTCACCACGCCCGAAGAGCTGGGCGAGCTGGCCGTGTTCTTCTGCTCCCCC
GCCGCCAACAACGTGCGCGGTGTTGCGTGGAACATGGACGGCGGCTGGGCGCAGTAA
SEQ ID NO: 30
Description: PhaZ
Alias: PhaZ_Ac, Q767A0, D(-)-3-hydroxybutyrate dehydrogenase, bhd
Length: 258
Type: Protein
Organism: Acidovorax sp. SA1
>MLKGKTALVTGSTSGIGLGIAKALARQGANIVLNGFGDVDGPRAEVLAAGEAAGAQVAYH
GADMSRVPHRDMMKYSASQFGRVDILVNNAGIQHVANVENFPVERWDSIIAINLTSALHT
SRLALPAMKSANWGRIINVASVHGLVGSAQKSAYVAAKHGIVGLTKVTALENATTGVTCN
AICPGWGLTPLVQKQVDAKAAEHGISNEEAKKLLLGEKEPSMQFTTPEELGELAVFFCSP
AANNVRGVAWNMDGGWAQ
SEQ ID NO: 31
Description: PhaY
Alias: PhaY_Ac, D-(-)-3-hydroxybutyrate oligomer hydrolase, Q9F1T8
Length: 876
Type: DNA
Organism: Acidovorax sp. SA1
>ATGACTCCGTTATCGCGCTACACCACCTGCGCTGGCTATGAAATCCATTACATGGAGTGG
GGCGCGCCGGATGCCCCGGTGGTGATCGCGTGGCACGGGCTGGCGCGCACCGGCCGCGAC
ATGGACCCGCTGGCCGCCCACCTGGCATCGCGCTACCGCGTGATCTGCCCCGACACATTG
GGACGGGGCCTGAGCCAGTGGGCGCGCGCGCCGCAGGACGAGTACCGCCTGTCGTTCTAC
GCGCGCATCGCGGCCGACCTGTTGGACCAGCTGGGCATCGAGAAAGCGCACTGGGTGGGC
ACATCGATGGGCGGGGCCATCGGTACGGTGTGCGCATCGGGATTGTTCGAGCCGCAGCTC
AAGGGCCGCGTCCAGAGCCTGCTGCTCAACGACAACGCCCCGCGCCTGGCCGACGCCGCG
CTGGAGCGCATCAAGGCCTATGCCGGCCACCCGCCTGCATTCGATACGGTGAAGGAACTG
GAGGCGTTCTTTCGGCAGGTATACAAGCCGTACGGCTGGCTCAGCGATGAGCAGTGGCGC
CTGCTCACCGAGAGCAGCACCCGCCGCCTGCCAGACGGTCGTGTCACGCCGCATTACGAC
CCCGCCATGGTGCAGCAGTTCACGCACCACACCAACGACTACCTGATCTGGGATCATTAC
GACGCGCTCGACATTCCCGTGTTGTGCCTGCGCGGGGCCGAGTCCGACTTGGTGCTGCCA
GACGTCACGGCCGAAATGTTGACCCGGGGCCCGGGATCACGTGGCCAGGCGCAGGTGGTG
GAGGTGGCCGGCTGCGGCCATGCCCCAGCGCTCAATGTGCCCGAGCACTACGCGCTGGTG
GACGGGTTTCTGGCCAGTGCGCAAGGCGGGCAATAG
SEQ ID NO: 32
Description: PhaY
Alias: PhaY_Ac, D-(-)-3-hydroxybutyrate oligomer hydrolase, Q9F1T8
Length: 291
Type: Protein
Organism: Acidovorax sp. SA1
>MTPLSRYTTCAGYEIHYMENGAPDAPVVIAWHGLARTGRDMDPLAAHLASRYRVICPDTL
GRGLSQWARAPQDEYRLSFYARIAADLLDQLGIEKAHWVGTSMGGAIGTVCASGLFEPQL
KGRVQSLLLNDNAPRLADAALERIKAYAGHPPAFDTVKELEAFFRQVYKPYGWLSDEQWR
LLTESSTRRLPDGRVTPHYDPAMVQQFTHHTNDYLINDHYDALDIPVLCLRGAESDLVLP
DVTAEMLTRGPGSRGQAQVVEVAGCGHAPALNVPEHYALVDGFLASAQGGQ
SEQ ID NO: 33
Description: lacZ alpha
Length: 814
Type: DNA
>ATGACCATGATTACGGATTCACTGGCCGTCGTTTTACAACGTCGTGACTGGGAAAACCCTGGCGTTACCCAAC
TTAATCGCCTTGCAGCACATCCCCCTTTCGCCAGCTGGCGTAATAGCGAAGAGGCCCGCACCGATCGCCCTTCC
CAACAGTTGCGCAGCCTGAATGGCGAATGGCGCTTTGCCTGGTTTCCGGCACCAGAAGCGGTGCCGGAAAGCTG
GCTGGAGTGCGATCTTCCTGAGGCCGATACTGTCGTCGTCCCCTCAAACTGGCAGATGCACGGTTACGATGCGC
CCATCTACACCAACGTGACCTATCCCATTACGGTCAATCCGCCGTTTGTTCCCACGGAGAATCCGACGGGTTGT
TACTCGCTCACATTTAATGTTGATGAAAGCTGGCTACAGGAAGGCCAGACGCGAATTATTTTTGATGGCGTTAA
CTCGGCGTTTCATCTGTGGTGCAACGGGCGCTGGGTCGGTTACGGCCAGGACAGTCGTTTGCCGTCTGAATTTG
ACCTGAGCGCATTTTTACGCGCCGGAGAAAACCGCCTCGCGGTGATGGTGCTGCGCTGGAATGACGGCAGTTAT
CTGGAAGATCAGGATATGTGGCGGATGAGCGGCATTTTCCGTGACGTCTCGTTGCTGCATAAACCGACTACACA
AATCAGCGATTTCCATGTTGCCACTCGCTTTAATGATGATTTCAGCCGCGCTGTACTGGAGGCTGAAGTTCAGA
TGTGCGGCGAGTTGCGTGACTACCTACGGGTAACAGTTTCTTTATGGCAGGGTGAAACGCAGGTCGCCAGCGGC
A
SEQ ID NO: 34
Description: Promoter and putative RBS
Alias: HP1
Length: 320
Type: DNA
>GACTAGGTCTTTCCCTTGCCGGAACAATCGGCTAAAGCCTTCCGCAGTCGGGGCGTAGCGCAGCCTGGTAGCG
CGACGGTTTTGGGTACCGTAGGTCGGAGGTTCGAATCCTCTCGCCCCGACCATCTTCGGGAAAACATTAATATT
TCCAGCGACGGAACGCGTGATGCGCCTGCCGCGTTCGGCGGCGAATGTCACGGATGATCCGCCTATGAGCCCTG
AACGCAGATGTCACGCGATGCCCTTTGGTCGCACCCCGATGGGCTGGTCATGCACCGCGCGGCAGCGTAGCCTG
TTCCCTGTCATATCAAGCAAGGGGC
SEQ ID NO: 35
Description: Promoter and RBS
Alias: pMxaF promoter
Length: 326
Type: DNA
Organism: M. extorquens
>GTTGACGACAACGGTGCGATGGGTCCCGGCCCCGGTCAAGACGATGCCAATACGTTGCGACACTACGCCTTGG
CACTTTTAGAATTGCCTTATCGTCCTGATAAGAAATGTCCGACCAGCTAAAGACATCGCGTCCAATCAAAGCCT
AGAAAATATAGGCGAAGGGACGCTAATAAGTCTTTCATAAGACCGCGCAAATCTAAGAATATCCTTAGATTCAC
GATGCGGCACTTCGGATGACTTCCGAGCGAGCCTGGAACCTCAGAAAAACGTCTGAGAGATACCGCGAGGCCGA
AAGGCGAGGCGGTTCAGCGAGGAGACGCAGG
SEQ ID NO: 36
Description: PhaZ1
Alias: PhaZ1_Re, Q0KCI0, Intracellular poly(3-hydroxybutyrate) depolymerase
Length: 1260
Type: DNA
Organism: Ralstonia eutropha
>ATGCTCTACCAATTGCATGAGTTCCAGCGCTCGATCCTGCACCCGCTGACCGCGTGGGCCCAGGCGACCGCCA
AGACCTTCACCAACCCCCTCAGCCCGCTCTCGCTGGTTCCCGGCGCACCCCGCCTGGCTGCCGGCTATGAACTG
CTGTACCGGCTCGGCAAGGAATACGAAAAGCCGGCATTCGACATCAAGTCGGTGCGCTCCAACGGGCGCGACAT
CCCCATCGTCGAGCAGACCGTGCTTGAAAAGCCGTTCTGCAAGCTGGTGCGCTTCAAGCGCTATGCCGACGACC
CGGAGACCATCAAGCTGCTCAAGGATGAGCCGGTGGTGCTGGTGGCCGCGCCGCTGTCGGGCCACCATGCCACG
CTGCTGCGCGACACGGTGCGCACGCTGCTGCAGGACCACAAGGTCTACGTCACCGACTGGATCGACGCACGCAT
GGTGCCGGTCGAGGAAGGCGCGTTCCACCTGTCGGACTACATCTACTACATCCAGGAATTCATCCGCCATATCG
GCGCCGAGAACCTGCATGTGATCTCGGTATGCCAGCCCACCGTGCCGGTGCTGGCCGCGATCTCGCTGATGGCC
TCGGCCGGCGAGAAGACGCCGCGCACCATGACCATGATGGGCGGCCCGATCGACGCCCGCAAGAGCCCCACCGC
GGTCAACTCGCTGGCGACCAACAAGTCGTTCGAGTGGTTCGAGAACAACGTCATCTACACCGTGCCGGCCAACT
ACCCCGGCCACGGCCGCCGCGTCTACCCGGGCTTTTTGCAGCATGCCGGTTTCGTGGCGATGAACCCGGACCGG
CACCTTTCCTCGCACTATGACTTCTACCTGAGCCTGGTCGAGGGCGATGCGGATGACGCCGAAGCCCACGTGCG
CTTCTACGACGAATACAACGCGGTGCTCGACATGGCCGCCGAGTACTACCTCGACACCATCCGCGAGGTGTTCC
AGGAATTCCGCCTGGCCAACGGCACCTGGGCCATCGACGGCAATCCGGTGCGGCCGCAGGACATCAAGAGCACC
GCGCTGATGACCGTCGAGGGCGAACTGGACGACATCTCGGGCGCGGGCCAGACCGCCGCGGCGCACGACCTGTG
CGCCGGCATCCCGAAAATCCGCAAGCAGCACCTGAACGCGGCACACTGCGGCCACTACGGCATCTTCTCGGGCC
GGCGCTGGCGCGAAGAGATCTACCCGCAGCTGCGCGACTTTATCCGCAAGTACCACCAGGCCTCGGCCACCAGG
TAA
SEQ ID NO: 37
Description: PhaZ1
Alias: PhaZ1_Re, Q0KCI0, Intracellular poly(3-hydroxybutyrate) depolymerase
Length: 419
Type: Protein
Organism: Ralstonia eutropha
>MLYQLHEFQRSILHPLTAWAQATAKTFTNPLSPLSLVPGAPRLAAGYELLYRLGKEYEKPAFDIKSVRSNGRD
IPIVEQTVLEKPFCKLVRFKRYADDPETIKLLKDEPVVLVAAPLSGHHATLLRDTVRTLLQDHKVYVTDWIDAR
MVPVEEGAFHLSDYIYYIQEFIRHIGAENLHVISVCQPTVPVLAAISLMASAGEKTPRTMTMMGGPIDARKSPT
AVNSLATNKSFEWFENNVIYTVPANYPGHGRRVYPGFLQHAGFVAMNPDRHLSSHYDFYLSLVEGDADDAEAHV
RFYDEYNAVLDMAAEYYLDTIREVFQEFRLANGTWAIDGNPVRPQDIKSTALMTVEGELDDISGAGQTAAAHDL
CAGIPKIRKQHLNAAHCGHYGIFSGRRWREEIYPQLRDFIRKYHQASATR
SEQ ID NO: 38
Description: PhaZ
Alias: PhaZ_Rp, P12625, ePHBDP, Extracellular Poly(3-hydroxybutyrate)
depolymerase
Length: 1467
Type: DNA
Organism: Ralstonia pickettii
>ATGGTGAGAAGACTGTGGCGACGGATCGCAGGCTGGCTcGCGGCCTGCGTGGCGATCTTGTGCGCGTTTCCAT
TGCATGCCGCCACGGCGGGGCCCGGTGCCTGGAGCAGCCAGCAGACCTGGGCGGCGGACTCCGTCAATGGCGGT
AACCTGACGGGCTACTTCTACTGGCCGGCCAGCCAGCCGACCACGCCGAATGGCAAGCGCGCGCTCGTCCTGGT
GCTGCACGGGTGCGTGCAGACGGCCTCGGGCGACGTCATCGACAACGCCAATGGCGCCGGCTTCAACTGGAAGT
CGGTCGCCGACCAGTATGGCGCCGTGATCCTGGCCCCGAATGCCACAGGGAACGTCTACAGCAACCATTGCTGG
GACTACGCAAACGCCTCGCCCAGCCGCACGGCCGGTCACGTCGGCGTCCTGCTGGACCTGGTCAATCGCTTCGT
CACCAATTCGCAGTACGCCATCGACCCCAACCAGGTCTACGTCGCCGGCTTGTCCTCgGGCGGCGGCATGACCA
TGGTGCTGGGCTGCATCGCGCCGGACATCTTCGCCGGCATCGGCATCAACGCTGGTCCcCCGCCGGGCACCACC
ACCGCGCAGATCGGCTACGTGCCGTCAGGCTTCACGGCGACGACGGCCGCGAACAAATGCAAtGCGTGGGCAGG
CTCCAACGCGGGCAAGTTCTCCACGCAGATCGCCGGTGCGGTCTGGGGAACCTCGGACTACACCGTGGCGCAGG
CGTATGGCCCGATGGATGCGGCGGCCATGCGTCTCGTCTACGGCGGCAACTTCACGCAGGGTTCGCAGGTcTCG
ATTTCGGGCGGCGGCACCAATACGCCGTACACCGACAGCAACGGCAAGGTGCGCACCCATGAGATCTCGGTCTC
CGGCATGGCCCACGCGTGGCCGGCCGGCACCGGCGGCGACAACACCAACTATGTCGATGCCACCCACATCAACT
ATCCGGTCTTCGTCATGGACTACTGGGTCAAGAACAACCTGCGCGCCGGCAGCGGGACGGGGCAGGCAGGCAGC
GCGCCGACCGGGCTTGCCGTCACGGCAACGACCTCCACGTCGGTCTCGCTGTCGTGGAATGCCGTCGCCAATGC
CAGCAGCTATGGCGTCTACCGCAACGGCAGCAAGGTCGGTTCGGCGACGGCCACCGCTTATACCGATTCCGGCC
TGATCGCCGGCACGACCTACAGCTACACGGTGACCGCGGTCGATCCGACGGCAGGCGAAAGCCAACCCTCCGCC
GCCGTATCGGCGACGACGAAATCGGCCTTCACCTGTACTGCCACCACGGCCAGCAACTACGCGCACGTGCAGGC
CGGGCGCGCGCACGACAGTGGCGGCATTGCCTACGCGAACGGCTCGAACCAGAGCATGGGGCTCGACAACCTCT
TCTACACGAGCACGCTGGCGCAGACGGCCGCCGGCTACTACATCGTCGGCAATTGTCCATGA
SEQ ID NO: 39
Description: PhaZ
Alias: PhaZ_Ac, Q767A0, D(-)-3-hydroxybutyrate dehydrogenase, bdh
Length: 777
Type: DNA
Organism: Acidovorax sp. SA1
>ATGCTGAAAGGCAAAACTGCCCTCGTCACCGGCTCCACCAGCGGCATTGGTCTTGGCATCGCCAAGGCCCTGG
CGCGCCAGGGCGCCAACATCGTGCTCAACGGCTTTGGCGATGTGGATGGCCCGCGTGCCGAAGTGCTGGCCGCC
GGTGAGGCCGCGGGGGCCCAGGTGGCGTACCACGGCGCGGACATGAGCCGTGTGCCACATCGAGACATGATGAA
GTACAGCGCCAGCCAGTTCGGCCGCGTGGACATCCTGGTCAACAACGCAGGCATCCAGCATGTGGCCAACGTGG
AGAACTTCCCCGTGGAGCGCTGGGATTCCATCATCGCCATCAACCTGACCAGCGCCTTGCACACCTCGCGCCTG
GCCCTGCCCGCGATGAAGAGCGCCAACTGGGGCCGCATCATCAATGTGGCGTCGGTGCACGGCCTGGTGGGATC
AGCCCAGAAGTCCGCCTATGTGGCGGCCAAGCACGGCATCGTGGGCCTGACCAAGGTCACTGCGCTGGAAAACG
CCACCACCGGCGTGACCTGCAATGCCATCTGCCCCGGCTGGGGTCTGACGCCACTGGTGCAAAAGCAGGTcGAT
GCCAAGGCCGCAGAACATGGCATCTCGAACGAAGAGGCCAAGAAGCTGTTGCTGGGCGAAAAGGAGCCTTCCAT
GCAGTTCACCACGCCCGAAGAGCTGGGCGAGCTGGCCGTGTTCTTCTGCTCCCCCGCCGCCAACAACGTGCGCG
GTGTTGCGTGGAACATGGACGGCGGCTGGGCGCAGTAA
SEQ ID NO: 40
Description: PhaY
Alias: PhaY_Ac, D-(-)-3-hydroxybutyrate oligomer hydrolase, Extracellular
3HB-oligomer hydrolase, e3HBOH, Q9X6X9
Length: 2226
Type: DNA
Organism: Ralstonia pickettii
>ATGAAAACGATACAAGGGAAGAGTCCGGGCCGCTGGTATTCGCGCGGCATGCTGCTGGCAGCGATGGCGGCGT
CCGGCGTCATCGGCCTGGCCGCGTGCGGTGGCGGCAATGATGGCAACTCAGCAGGCAACAATGGCAATGCCGGA
GGCAACGGCAACAACAACGGCAACAACAACGGCAATACGGTGAGCAACACCAAGCCGTCCTTCGTCGGTACCGT
GACGGTCAGGCGATTCGACGGTGTGAGCGACGACTTGCTGACCGCGGGCCTGGGCGCCTCCGGGCTGGCTTCGG
CCACGGCgCCTGCCGTGGCCAACGCCGTTGCGCCGACCGCGGCAGAGTTGCGGCGCCTGACCATCTACAACAAC
TATCGCGCCTTGATCGACACCAGCGCCAAGGGTGGCTACGGCACACTCTACGGCCCCAACGTCGATGCCGACGG
CAACGTCACCTCCGGCAATGGCATGGTGGCCGGCGCGGAGTATGTCGCGTACCCGGATGACGGCTCCGGCCAGC
AGAACGTGGTGCTGCTGGTGCAGATTCCCGACGCATTCGATGCaGCGCATCCGTGCATCATCACCGCGACCTCG
TCGGGTTCGCGCGGCATCTACGGGGCCATTTCGACCGGTGAGTGGGGACTCAAGCGCAAGTGCGCGGTCGCCTA
TACCGATAAGGGTACCGGCGCCGGCCCGCAtGACCTGGCCACCGACACGGTGCCGCTGCAGGAtGGCACGCGCA
CGACACGCACGCTCGCCGGCAACACGGCGCAATTCGCaGCGCCGCTCGCCGCGAGCCGGCTTGCaGCCTTCAAC
GTGGCAACGCCCAACCGGCTGGCGTTCAAGCATGCGCACTCGCAGCGCAACCCCGAGAAGGACTGGGGCCTCTT
CACGCTGCAGGCGGTGCAGTTCGCCTTCTGGGCCATCAACGACAAGCTGGGCATCTCCAGCGGGCAGACCGTCA
GCCAGTTGCCGGTGCGTCCCGGCAACACCATCGTGATCGCTTCCAGTGTGTCCAATGGTGGCGGCGCGGCGATC
GCGGCGGCCGAGCAGGACACCGGCAACCTGATCGATGGCGTGGCGGTtGGCGAGCCCGCATTGAGCCTGCCGTC
CTCGATCAACGTGCAGGTCAAGCGCGGCGGCGCAAGCTTGCCGATCAAtGGCAAGCCGCTGTTCGACTACGTCA
GCTATGCCAACGAATTCCGGCTGTGCGCGGCGCTGTCGGCCAGCGTGGCAAGCGCGCCGACGCAGGCTTACTTT
GGAGCGGCTTTAGGCTGGCCCGCCAGCGTGCAGGCGAACCGtTGCGCAGCGCTGCACGCCAAGGGCCTGTTGTC
GTCCACCACCACGGCAGCACAGGCCGACGAGGCGCTGCAGAAGATGCGCGACTACGGTTGGGAGCCCGAATCCG
ACCTCCTGCATGCCTCCATGGCGTACTTCGAGATCGATCCGTCGGTCGCCACCACCTTCGGCAACGCCCTGGCG
CGCGCCAGCGTGTTCGACAATCTGTGCGACCTCAGCTTTGCGGCGGTGGATGGCTCGTTCCACCCGGCCACGAT
GAACGCCACGGTGCTGGCGCAACTGGCaGCCACCGGCAACGGAGTTCCTCCCACGACCGGCGTGCAGTTGATCA
ACAATATTGCCCAGGGTGGTGCGGCGCAGAGCAGGCAGTCGATCGACTCCTCCGGTACGCAGGCCGCCAACCTG
GATGGCGCGCTATGCCTGCGCAACCTGCTGAGCGGCAGCGACGCCGCCTCGCAGGCGCTGCAGCTTGGCCTGTC
GCAGACGCTGCGCAGCGGCAATCTGCGCGGCAAGCCAGCCCTGATCGTGCAAGGGCGGAACGATGCCCTGCTGC
CGGTCAACCATGGCGCTCGCCCGTATCTGGGCCTCAATGCGCAGGTCGATGGGAGCAGCAAGCTGTCGTATATC
GAGGTCACGAACGCCCAGCACTTCGATGGCTTCATTGATCTGTTGCCGGGATACGACTCGCTCTTCGTGCCCTT
GGCCGTCTATGAGCAACGCGCGCTTGACGCCGTGTACGCGAACCTGAGGAGCGGCACGCCGCTGCCACCGTCGC
AAGTGGTACGCACGACGCCGCGCGGCGGTGCGGCAGGAGCGGCGCCGCCTATCACGGCgGCCAACGTGCCGAAC
TTCACCATGACTCCGGCTGCGGGTGACCGTATCCAGGTGAGCGTCTCGGGTGGCGTGGCGACGGTTTCCGTGCC
GAACTGA
SEQ ID NO: 41
Description: PhaY
Alias: D-(-)-3-hydroxybutyrate oligomer hydrolase, Q9F1T8
Length: 876
Type: DNA
Organism: Acidovorax sp. SA1
>ATGACTCCGTTATCGCGCTACACCACgTGCGCTGGCTATGAAATCCATTACATGGAGTGGGGtGCGCCGGATG
CCCCGGTGGTGATCGCaTGGCAtGGGCTGGCGCGCACCGGCCGCGACATGGACCCGCTGGCCGCCCAtCTGGCA
TCGCGCTACCGCGTGATCTGCCCCGACACATTGGGACGGGGCCTGAGCCAGTGGGCGCGCGCGCCGCAGGACGA
GTACCGCCTGTCGTTCTACGCGCGCATCGCGGCCGACCTGTTGGACCAGCTGGGCATCGAGAAAGCGCACTGGG
TGGGCACATCGATGGGCGGGGCCATCGGTACGGTGTGCGCATCGGGATTGTTCGAGCCGCAGCTCAAGGGCCGC
GTCCAGAGCCTGCTGCTCAACGACAACGCCCCGCGCCTGGCCGACGCCGCGCTGGAGCGCATCAAGGCCTATGC
CGGCCACCCGCCTGCATTCGATACGGTGAAGGAACTGGAGGCGTTCTTTCGGCAGGTATACAAGCCGTACGGCT
GGCTCAGCGATGAGCAGTGGCGCCTGCTCACCGAGAGCAGCACCCGCCGCCTGCCAGACGGTCGTGTCACGCCG
CATTACGACCCCGCCATGGTGCAGCAGTTCACGCACCACACCAACGACTACCTGATCTGGGATCATTACGACGC
GCTCGACATTCCCGTGTTGTGCCTGCGCGGGGCCGAGTCCGACTTGGTGCTGCCAGACGTCACGGCCGAAATGT
TGACCCGGGGCCCGGGATCACGTGGtCAGGCGCAGGTcGTGGAGGTGGCaGGCTGCGGCCATGCCCCAGCGCTC
AATGTGCCCGAGCACTACGCGCTGGTGGACGGGTTTCTGGCCAGTGCGCAAGGCGGGCAATGA
SEQ ID NO: 42
Description: PhaM
Alias: PhaM_Rp
Length: 792
Type: DNA
Organism: Ralstonia eutropha
>ATGTTCGGACAGATTCCCGATTTCACCAACGGCTTCGACTTCATGCGCCGGCTGTGGGGCAGCGGCAGCGGCA
TGCCCGCCGGCATGATGCCCGGCCTGCAGGCAATGACGCCGCCGATGGACCTCGACGACCTCGACAAGCGCATT
GCCGACCTGAAGGCCGTGGAGAGCTGGCTGCAACTGAACACCAACCTGCTGCGCACCACCATCCAGGGCCTGGA
AGTGCAGCGCGCCACGCTGGTGGCGCTGCAGACCTTCGGCAACGCGCTGTCGCCCGAGGCCATGCAGTCCGCCA
TGGAAAACGTCGCGCGCGCGGCCAACACGCCCAGCGCCGCAGCGCCGGAGCGCGACGCCGGCGCGGATGCCGAC
AGCGGCACGCAACAAGAGCCGCCTGCCGCCGAGCGCCCGCAAGCTGCGGCAAGCGACACCGATTCCGCCTTGCC
GCCCAACGCCGCGCTGTGGTGGGACCTGCTGCAACAGCAGTTCAACCAGATCGCCAGCAGCGCCGCCGCGGCCA
GCATCGCCCCGTTCGGCATGGGCGGCGTGGGCGGCTTCGGCACCGCAGCCAGCCCCGACGCCGCAGCGCAAGCG
GCGGCTGCAAAACCGAAGACGGACGCGCCTGGCAAGGCCGCCTCCGCCGGCACGGGCAAGCCCGCCGCCAGGAA
AGCGCCGGCAAAGAAGGCGCCGGCCAAAAAGGCGGCCAAGGCAAAACCGGCCAGGGACGCCGGCAACGGCGAGG
ACAATGGCAAGAACGGGGGCAACAACGGCGCCAACGGCAGCAGCGCAGCCTGA
SEQ ID NO: 43
Description: PhaM
Alias: PhaM_Rp
Length: 263
Type: Protein
Organism: Ralstonia eutropha
>MFGQIPDFTNGFDFMRRLWGSGSGMPAGMMPGLQAMTPPMDLDDLDKRIADLKAVESWLQLNTNLLRTTIQGL
EVQRATLVALQTFGNALSPEAMQSAMENVARAANTPSAAAPERDAGADADSGTQQEPPAAERPQAAASDTDSAL
PPNAALWWDLLQQQFNQIASSAAAASIAPFGMGGVGGFGTAASPDAAAQAAAAKPKTDAPGKAASAGTGKPAAR
KAPAKKAPAKKAAKAKPARDAGNGEDNGKNGGNNGANGSSAA
SEQ ID NO: 44
Description: PhaZ7, Q939Q9
Alias: PhaZ7_P1
Length: 1143
Type: DNA
Organism: Paucimonas lemoignei
>ATGATTTCAAAACTCATGGGAGCGCAGCGCTTCCTTCCGGCAGTCGCTGCCACGGTGACTTCCCTCGTCTGGG
GCCTGGCCGGCACCCTCGTCGCTCCTGGCGAGGCGCAGGCGCTGACTTGCGGCACCAACAGTGGCTTTGTCTGC
AAGGGCACGCAAACCCAATACGCCGGCGGCTTCGCTCCGGGCGTGGGTTACGGCGGGTTTGGCGGCGGTTCTTG
CACGGCCACGAAAACCCCGGTGATTTTCATTCACGGTAACGGCGACAATGCCATCAGTTTCGATATGCCcCCGG
GTAATGTCAGCGGCTACGGCACGCCGGCCCGCTCGGTCTATGCCGAGTTGAAAGCGCGCGGCTATAACGATTGC
GAGATCTTCGGCGTCACCTACCTGAGCTCCAGCGAGCAGGGTTCCGCGCAATACAACTATCACTCATCGACCAA
GTACGCGATCATCAAAACCTTCATCGACAAGGTCAAGGCTTATACCGGCAAATCCCAAGTGGACATCGTTGCCC
ACTCCATGGGCGTGTCGATGTCGCTGGCGACGCTCCAGTACTACAACAACTGGACCAGCGTGCGCAAGTTCATC
AACCTCGCCGGCGGCATCCGCGGCTTGTATAGCTGCTATTACACCGGCTACGCCAATGCGGCAGCGCCGACTTG
CGGTTCGCAAAACTATTACAACAGCTATACCTTCGGCTTTTTCCCTGAAGGCTGGTATTACGGCGTCTGGGTGA
GCAACCCATGGACCGGCAGCGGCTCGACCAATTCGATGCGCGACATGCCGGCCAAGCGCACCGCGGTATCGTTC
TACACCCTGTCGGCAGGGTTCAAGGATCAGGTCGGTTGCGCCACGGCGTCGTTCTGGGCTGGTTGCGATTCTGC
CGCCAAGTTCGCCAGCACGACTTCCAACGTGAAAGCGCAAATCAATGTCGGCGCGGGAAGCAATGCTACCCAGG
CCGATTACGACTGGGCCGACGGCATGCCGTACAACGCGGGCGGCGGCGACACCACCAACGGTGTCGGCCACTTC
CGCACCAAGACCAATACCGGCGCCATCATCCAGCGCATGCTGCTGACCACTTGTACCGGCCTGGATTGCGCAGC
CGAATACACGACCGGTCCTAAAGCCGCGTACTAA
SEQ ID NO: 45
Description: PhaZ7, Q939Q9
Alias: PhaZ7_P1
Length: 380
Type: Protein
Organism: Paucimonas lemoignei
>MISKLMGAQRFLPAVAATVTSLVWGLAGTLVAPGEAQALTCGTNSGFVCKGTQTQYAGGFAPGVGYGGFGGGS
CTATKTPVIFIHGNGDNAISFDMPPGNVSGYGTPARSVYAELKARGYNDCEIFGVTYLSSSEQGSAQYNYHSST
KYAIIKTFIDKVKAYTGKSQVDIVAHSMGVSMSLATLQYYNNWTSVRKFINLAGGIRGLYSCYYTGYANAAAPT
CGSQNYYNSYTFGFFPEGWYYGVWVSNPWTGSGSTNSMRDMPAKRTAVSFYTLSAGFKDQVGCATASFWAGCDS
AAKFASTTSNVKAQINVGAGSNATQADYDWADGMPYNAGGGDTTNGVGHFRTKTNTGAIIQRMLLTTCTGLDCA
AEYTTGPKAAY

1. A non-naturally occurring microorganism that produces about 1% to about 99.9% less of a polyhydroxyalkanoate (PHA) product by weight and about 1% to about 250% more protein by weight than the parent microorganism from which the non-naturally occurring microorganism is derived.

2. A non-naturally occurring microorganism according to claim 1, wherein the weight ratio of PHA to protein produced by the microorganism comprises about 1:1000 to about 2:1.

3. A non-naturally occurring microorganism according to claim 2, wherein the weight ratio of PHA to protein produced by the microorganism comprises about 1:1000 to about 1:6.

4. A non-naturally occurring microorganism according to claim 1, wherein the PHA product comprises poly-β-hydroxybutyrate (PHB).

5. A non-naturally occurring microorganism according to claim 1, wherein the microorganism is of the genus Methylomonas, Methylobacter, Methylococcus, Methylosinus, Methylocyctis, Methylomicrobium, Methanomonas, Methylophilus, Methylobacillus, Methylobacterium, Hyphomicrobium, Xanthobacter, Bacillus, Paracoccus, Nocardia, Arthrobacter, Rhodopseudomonas, Pseudomonas, Candida, Hansenula, Pichia, Torulopsis, Vibrio, Escherichia, Alcaligenes, Ralstonia, Rhodobacter, Saccharomyces, Cupriavidus, Sinorhizobium, Mucor, Bradyrhizobium, Yarrowia, Azotobacter, Synechocystis, Rhodotorula, Aeromonas, Magnetospirillum, Haloferax, Caryophanon, or Allochromatium.

6. A non-naturally occurring microorganism according to claim 5, wherein the microorganism is a Methylobacterium.

7. A non-naturally occurring microorganism according to claim 6, wherein the microorganism is Methylobacterium extorquens.

8. A non-naturally occurring microorganism according to claim 1, wherein said microorganism or the parent microorganism from which it is derived is genetically modified or artificially pre-selected to produce elevated levels of one or more carotenoid compound(s) relative to the corresponding unmodified or unselected microorganism.

9. A non-naturally occurring microorganism according to claim 8, wherein said carotenoid compound(s) are selected from β-carotene, lycopene, rhodopsin, zeaxanthin, lutein, canthaxanthin, phoenicoxanthin, echinenone, cryptoxanthin, astaxanthin, adinoxanthin, 3-hydroxyechinenone, and sprilloxanthin.

10. A non-naturally occurring microorganism according to claim 1, wherein the PHA is in intracellular granule(s).

11. A non-naturally occurring microorganism according to claim 1, comprising mutation(s) in one or more endogenous PHA biosynthesis gene(s), PHA degradation gene(s), and/or phasin gene(s), or external regulatory sequence(s) thereof, resulting in reduced production of PHA.

12. A non-naturally occurring microorganism according to claim 11, wherein the mutation(s) comprises deletion or reduced expression of one or more PHA biosynthesis gene(s), or results in reduced enzymatic activity of one or more PHA biosynthetic enzyme(s).

13. A non-naturally occurring microorganism according to claim 12, wherein the one or more PHA biosynthesis gene(s) comprises phaA, phaB, and/or phaC.

14. A non-naturally occurring microorganism according to claim 11, wherein the mutation(s) comprises enhanced expression of one or more PHA degradation gene(s) or results in enhanced enzymatic activity of one or more PHA degradation enzyme(s).

15. A non-naturally occurring microorganism according to claim 14, wherein the one or more PHA degradation enzyme(s) comprises phaY, phaZ, and/or hbd.

16. A non-naturally occurring microorganism according to claim 11, wherein the mutation(s) comprises deletion or reduced expression of one or more phasin gene(s), or results in reduced binding affinity of one or more phasin(s) for intracellular PHA granules.

17. A non-naturally occurring microorganism according to claim 16, wherein the one or more phasin(s) comprises Mext_2223, Mext_2560, and/or Mext_0493.

18. A non-naturally occurring microorganism according to claim 1, comprising one or more heterologous PHA degradation gene(s), resulting in reduced production of PHA or PHA with altered polymer molecular weight distribution or PHA with polymers that have reduced molecular weight on average or increased digestibility.

19. A non-naturally occurring microorganism according to claim 18, wherein the one or more heterologous PHA degradation gene(s) comprises phaY and/or phaZ.

20. A feed or nutritional supplement composition comprising the non-naturally occurring microorganism of claim 1.

21.-23. (canceled)

24. A feed or nutritional supplement composition comprising a plurality of non-naturally occurring microorganisms according to claim 1, each comprising mutation(s) in one or more PHA biosynthesis gene(s) and/or mutation(s) in one or more phasin(s), wherein each of said plurality of non-naturally occurring microorganisms produces PHA and protein at a different level, wherein the combination of non-naturally occurring microorganisms provides PHA and protein in the composition at a weight ratio of about 1:1000 to about 2:1.

25. A method for producing biomass, comprising culturing a microorganism that produces PHA in a culture medium under conditions suitable for growth of the microorganism, wherein the culture conditions result in biomass comprising PHA:protein in a weight ratio of about 1:1000 to about 2:1.

26. A method for producing biomass according to claim 25, wherein the culture conditions result in biomass comprising PHA:protein in a weight ratio of about 1:1000 to about 1:6.

27.-30. (canceled)

31. A method according to claim 25, wherein the culture conditions comprise one or more alcohol(s) as a carbon source for producing said biomass.

32. (canceled)

33. A method according to claim 25, wherein the culture conditions comprise aeration of the culture medium, resulting in dissolved oxygen in the culture medium of about 5% to about 50%.

34. (canceled)

35. A method according to claim 25, wherein the culture conditions comprise a temperature of about 20° C. to about 50° C.

36. A method according to claim 25, wherein the culture conditions comprise removal of a portion of about 10% to about 90% of the culture medium when the culture reaches an optical density measured at 600 nm of about 50 to about 200, followed by replacement with an equivalent amount of fresh medium, thereby maintaining PHA production at a relatively constant level.

37. A method according to claim 25, wherein the culture conditions comprise continuous removal of culture medium and microorganisms and continuous replenishment with fresh culture medium.

38.-42. (canceled)

43. A method according to claim 25, wherein the microorganism is a non-naturally occurring microorganism that produces about 99.9% to about 1% less of a polyhydroxyalkanoate (PHA) product by weight and about 1% to about 250% more protein by weight than the parent microorganism from which the non-naturally occurring microorganism is derived.

44.-45. (canceled)

46. A method for improving survivability of a livestock or aquaculture animal, comprising feeding said animal a feed composition that comprises biomass produced in a method according to claim 25, wherein the feed composition comprises PHA:protein in a weight ratio of about 1:1000 to about 2:1, and wherein the survivability is increased by at least about 1% in comparison to a feed composition that comprises no PHA.

47. A method according to claim 46, wherein the feed composition comprises PHA:protein in a weight ratio of about 1:1000 to about 1:6.

48. (canceled)

49. A method according to claim 46, wherein the feed composition comprises a plurality of microorganisms, wherein each of said plurality of microorganisms produces PHA and protein at a different level, wherein the combination of microorganisms provides PHA and protein in the composition at a weight ratio of about 1:1000 to about 2:1.

50.-54. (canceled)

55. A method according to claim 31, wherein the culture conditions comprise one or more additional carbon source(s) producing said biomass.

56. A method according to claim 55, wherein said carbon source(s) comprise formate, acetate, malate, succinate, or a combination thereof.
